SEQUENCE LISTING (130 sequences)

SEQ ID NO: 1 - DNA, 804 bp, Artificial Sequence
  synthetic nucleic acid
atggagtttgggctgagctggctttttcttgtggctattttaaaaggtgtccagtgctctagagacatccagatgacacagagccctagcagcctgtctgccagcgtgggagacagagtgaccatcacctgtcaggcctctcagaacgtgcggacagccgtggcttggtatcagcagaagcctggcaaggcccctaaggctctgatctacctgcctagcaacagactgaccggcgtgccctctagattcagcggctctggctctggcaccgacttcaccttcacaatcagcagcctgcagcctgaggatatcgctacctacttctgcctgcaacactggaactaccctttcaccttcggccagggcaccaagctggaaatcaagggtggtggtggttctggtggtggtggttctggcggcggcggctccggtggtggtggatccgaggttcagctgcagcagtctggacctgagctggttaagcctggcgcctccgtgaagatcccttgcaagacaagcggctacatcttcaccagctactggatccactgggtcaagcagagccacggcaagagcctggaatggatcggcagaatctaccccggcgtggacagcatctactacaacgagaagttcaagggcaaagccacactgaccgccgacaagagcagcagcacagcctacatggaactgagaagcctgaccagcgaggataccgccgtgtacttctgtgccaccatcagctacgactacgattacagcatggactactggggccagggcaccaccgtgacagtttcttct

SEQ ID NO: 2 - protein, 268 aa, Artificial Sequence
  synthetic protein
MEFGLSWLFLVAILKGVQCSRDIQMTQSPSSLSASVGDRVTITCQASQNVRTAVAWYQQKPGKAPKALIYLPSNRLTGVPSRFSGSGSGTDFTFTISSLQPEDIATYFCLQHWNYPFTFGQGTKLEIKGGGGSGGGGSGGGGSGGGGSEVQLQQSGPELVKPGASVKIPCKTSGYIFTSYWIHWVKQSHGKSLEWIGRIYPGVDSIYYNEKFKGKATLTADKSSSTAYMELRSLTSEDTAVYFCATISYDYDYSMDYWGQGTTVTVSS

SEQ ID NO: 3 - DNA, 321 bp, Artificial Sequence
  synthetic nucleic acid
gacatccagatgacacagagccctagcagcctgtctgccagcgtgggagacagagtgaccatcacctgtcaggcctctcagaacgtgcggacagccgtggcttggtatcagcagaagcctggcaaggcccctaaggctctgatctacctgcctagcaacagactgaccggcgtgccctctagattcagcggctctggctctggcaccgacttcaccttcacaatcagcagcctgcagcctgaggatatcgctacctacttctgcctgcaacactggaactaccctttcaccttcggccagggcaccaagctggaaatcaag

SEQ ID NO: 4 - protein, 107 aa, Artificial Sequence
  synthetic protein
DIQMTQSPSSLSASVGDRVTITCQASQNVRTAVAWYQQKPGKAPKALIYLPSNRLTGVPSRFSGSGSGTDFTFTISSLQPEDIATYFCLQHWNYPFTFGQGTKLEIK

SEQ ID NO: 5 - protein, 11 aa, Artificial Sequence
  synthetic protein
QASQNVRTAVA

SEQ ID NO: 6 - protein, 7 aa, Artificial Sequence
  synthetic protein
LPSNRLT

SEQ ID NO: 7 - protein, 9 aa, Artificial Sequence
  synthetic protein
LQHWNYPFT

SEQ ID NO: 8 - DNA, 360 bp, Artificial Sequence
  synthetic nucleic acid
gaggttcagctgcagcagtctggacctgagctggttaagcctggcgcctccgtgaagatcccttgcaagacaagcggctacatcttcaccagctactggatccactgggtcaagcagagccacggcaagagcctggaatggatcggcagaatctaccccggcgtggacagcatctactacaacgagaagttcaagggcaaagccacactgaccgccgacaagagcagcagcacagcctacatggaactgagaagcctgaccagcgaggataccgccgtgtacttctgtgccaccatcagctacgactacgattacagcatggactactggggccagggcaccaccgtgacagtttcttct

SEQ ID NO: 9 - protein, 118 aa, Artificial Sequence
  synthetic protein
QLQQSGPELVKPGASVKIPCKTSGYIFTSYWIHWVKQSHGKSLEWIGRIYPGVDSIYYNEKFKGKATLTADKSSSTAYMELRSLTSEDTAVYFCATISYDYDYSMDYWGQGTTVTVSS

SEQ ID NO: 10 - protein, 10 aa, Artificial Sequence
  synthetic protein
GYIFTSYWIH

SEQ ID NO: 11 - protein, 17 aa, Artificial Sequence
  synthetic protein
RIYPGVDSIYYNEKFKG

SEQ ID NO: 12 - protein, 13 aa, Artificial Sequence
  synthetic protein
ATISYDYDYSMDY

SEQ ID NO: 13 - DNA, 798 bp, Artificial Sequence
  synthetic nucleic acid
atggagtttgggctgagctggctttttcttgtggctattttaaaaggtgtccagtgctctagagatgttgtgctaactcagtctccagccaccctgtctgtgactccaggagatagcgtcagtctttcctgcagggccagccaaagtattagcatcaacctacactggtatcaacaaaaatcacatgagtctccaaggcttctcattaagtatgcttcccagtccatctctgggatcccctccaggttcagtggcagtggatcagggacagatttcactctcagtatccacagtgtggagactgaagattttggaatgtttttctgtcaacacagtaacagctggcctctcacgttcggtgctgggaccaagctggaaatcaaaggtggtggtggttctggtggtggtggttctggcggcggcggctccggtggtggtggatcccaggttactctgaaagagtctggccctgggatattgcagccctcccagaccctcagtctgacttgttctttctctgggttttcactgagcacttctggtatgggtgttggctggattcgtcagccatcacggaagggtctggagtggctggcacacatttggtgggatgatgtcaagcgctataacccagccctgaagagccgactgactatctccaaggatacctccagcagccaggtgttcctcaagatcgccagtgtggacactgcggatactgccacatactactgtgctcgaataccccactggtacttcgatgtctggggcgcagggaccacggtcaccgtctcctca

SEQ ID NO: 14 - protein, 266 aa, Artificial Sequence
  synthetic protein
MEFGLSWLFLVAILKGVQCSRDVVLTQSPATLSVTPGDSVSLSCRASQSISINLHWYQQKSHESPRLLIKYASQSISGIPSRFSGSGSGTDFTLSIHSVETEDFGMFFCQHSNSWPLTFGAGTKLEIKGGGGSGGGGSGGGGSGGGGSQVTLKESGPGILQPSQTLSLTCSFSGFSLSTSGMGVGWIRQPSRKGLEWLAHIWWDDVKRYNPALKSRLTISKDTSSSQVFLKIASVDTADTATYYCARIPHWYFDVWGAGTTVTVSS

SEQ ID NO: 15 - DNA, 321 bp, Artificial Sequence
  synthetic nucleic acid
gatgttgtgctaactcagtctccagccaccctgtctgtgactccaggagatagcgtcagtctttcctgcagggccagccaaagtattagcatcaacctacactggtatcaacaaaaatcacatgagtctccaaggcttctcattaagtatgcttcccagtccatctctgggatcccctccaggttcagtggcagtggatcagggacagatttcactctcagtatccacagtgtggagactgaagattttggaatgtttttctgtcaacacagtaacagctggcctctcacgttcggtgctgggaccaagctggaaatcaaa

SEQ ID NO: 16 - protein, 107 aa, Artificial Sequence
  synthetic protein
DVVLTQSPATLSVTPGDSVSLSCRASQSISINLHWYQQKSHESPRLLIKYASQSISGIPSRFSGSGSGTDFTLSIHSVETEDFGMFFCQHSNSWPLTFGAGTKLEIK

SEQ ID NO: 17 - protein, 11 aa, Artificial Sequence
  synthetic protein
RASQSISINLH

SEQ ID NO: 18 - protein, 7 aa, Artificial Sequence
  synthetic protein
YASQSIS

SEQ ID NO: 19 - protein, 9 aa, Artificial Sequence
  synthetic protein
QHSNSWPLT

SEQ ID NO: 20 - DNA, 354 bp, Artificial Sequence
  synthetic nucleic acid
caggttactctgaaagagtctggccctgggatattgcagccctcccagaccctcagtctgacttgttctttctctgggttttcactgagcacttctggtatgggtgttggctggattcgtcagccatcacggaagggtctggagtggctggcacacatttggtgggatgatgtcaagcgctataacccagccctgaagagccgactgactatctccaaggatacctccagcagccaggtgttcctcaagatcgccagtgtggacactgcggatactgccacatactactgtgctcgaataccccactggtacttcgatgtctggggcgcagggaccacggtcaccgtctcctca

SEQ ID NO: 21 - protein, 118 aa, Artificial Sequence
  synthetic protein
QVTLKESGPGILQPSQTLSLTCSFSGFSLSTSGMGVGWIRQPSRKGLEWLAHIWWDDVKRYNPALKSRLTISKDTSSSQVFLKIASVDTADTATYYCARIPHWYFDVWGAGTTVTVSS

SEQ ID NO: 22 - protein, 12 aa, Artificial Sequence
  synthetic protein
GFSLSTSGMGVG

SEQ ID NO: 23 - protein, 16 aa, Artificial Sequence
  synthetic protein
HIWWDDVKRYNPALKS

SEQ ID NO: 24 - protein, 10 aa, Artificial Sequence
  synthetic protein
ARIPHWYFDV

SEQ ID NO: 25 - DNA, 804 bp, Artificial Sequence
  synthetic nucleic acid
atggagtttgggctgagctggctttttcttgtggctattttaaaaggtgtccagtgctctagagacattgtgatgacccagtctcaaaaattcatgtccacatcagtaggagacagggtcagcatcacctgcaaggccagtcagaatgttcgtactgctgtagcctggtatcaacagaaaccaggccagtctcctaaagcactgatttacttgccatccaaccggctcactggagtccctgatcgcttcacaggcagtggatctgggacagatttcactctcaccattagcaatgtccaatctgaagacctggcagattatttctgtctgcaacattggaattatccattcacgttcggctcggggacaaagttggaaataaaaggtggtggtggttctggtggtggtggttctggcggcggcggctccggtggtggtggatcccaggtccagctgaagcagtctggagctgagctggtgaggcctggggcttcagtgaatctgtcctgcaagacttctggatacatcttcaccagctactggattcactgggtaaaacagaggtctggacagggccttgagtggattgcaaggatttatcctggagttgatagtatttattacaatgagaagttcaagggcaaggccacactgactgcagacaaatcctccagcactgcctacatgcagctcagcagcctgaaatctgaggactctgctgtctatttctgtgcaactatctcctatgattacgactattctatggactactggggtcaaggaacctcagtcaccgtctcctca

SEQ ID NO: 26 - protein, 268 aa, Artificial Sequence
  synthetic protein
MEFGLSWLFLVAILKGVQCSRDIVMTQSQKFMSTSVGDRVSITCKASQNVRTAVAWYQQKPGQSPKALIYLPSNRLTGVPDRFTGSGSGTDFTLTISNVQSEDLADYFCLQHWNYPFTFGSGTKLEIKGGGGSGGGGSGGGGSGGGGSQVQLKQSGAELVRPGASVNLSCKTSGYIFTSYWIHWVKQRSGQGLEWIARIYPGVDSIYYNEKFKGKATLTADKSSSTAYMQLSSLKSEDSAVYFCATISYDYDYSMDYWGQGTSVTVSS

SEQ ID NO: 27 - DNA, 321 bp, Artificial Sequence
  synthetic nucleic acid
gacattgtgatgacccagtctcaaaaattcatgtccacatcagtaggagacagggtcagcatcacctgcaaggccagtcagaatgttcgtactgctgtagcctggtatcaacagaaaccaggccagtctcctaaagcactgatttacttgccatccaaccggctcactggagtccctgatcgcttcacaggcagtggatctgggacagatttcactctcaccattagcaatgtccaatctgaagacctggcagattatttctgtctgcaacattggaattatccattcacgttcggctcggggacaaagttggaaataaaa

SEQ ID NO: 28 - protein, 107 aa, Artificial Sequence
  synthetic protein
DIVMTQSQKFMSTSVGDRVSITCKASQNVRTAVAWYQQKPGQSPKALIYLPSNRLTGVPDRFTGSGSGTDFTLTISNVQSEDLADYFCLQHWNYPFTFGSGTKLEIK

SEQ ID NO: 29 - protein, 11 aa, Artificial Sequence
  synthetic protein
KASQNVRTAVA

SEQ ID NO: 30 - protein, 7 aa, Artificial Sequence
  synthetic protein
LPSNRLT

SEQ ID NO: 31 - protein, 9 aa, Artificial Sequence
  synthetic protein
LQHWNYPFT

SEQ ID NO: 32 - DNA, 360 bp, Artificial Sequence
  synthetic nucleic acid
caggtccagctgaagcagtctggagctgagctggtgaggcctggggcttcagtgaatctgtcctgcaagacttctggatacatcttcaccagctactggattcactgggtaaaacagaggtctggacagggccttgagtggattgcaaggatttatcctggagttgatagtatttattacaatgagaagttcaagggcaaggccacactgactgcagacaaatcctccagcactgcctacatgcagctcagcagcctgaaatctgaggactctgctgtctatttctgtgcaactatctcctatgattacgactattctatggactactggggtcaaggaacctcagtcaccgtctcctca

SEQ ID NO: 33 - protein, 120 aa, Artificial Sequence
  synthetic protein
QVQLKQSGAELVRPGASVNLSCKTSGYIFTSYWIHWVKQRSGQGLEWIARIYPGVDSIYYNEKFKGKATLTADKSSSTAYMQLSSLKSEDSAVYFCATISYDYDYSMDYWGQGTSVTVSS

SEQ ID NO: 34 - protein, 10 aa, Artificial Sequence
  synthetic protein
GYIFTSYWIH

SEQ ID NO: 35 - protein, 17 aa, Artificial Sequence
  synthetic protein
RIYPGVDSIYYNEKFKG

SEQ ID NO: 36 - protein, 13 aa, Artificial Sequence
  synthetic protein
ATISYDYDYSMDY

SEQ ID NO: 37 - DNA, 807 bp, Artificial Sequence
  synthetic nucleic acid
atggagtttgggctgagctggctttttcttgtggctattttaaaaggtgtccagtgctctagacaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgcctgcagtggagtagtaacccgctcacgttcggtgctgggaccaagctggaaatcaaaggtggtggtggttctggtggtggtggttctggcggcggcggctccggtggtggtggatccgaggtccagctgcagcagtctggacctgagctggtgaagcctggggcttcagtgaagatatcctgcaagacttctggatacacattcactgaatacaccatgcactgggtgaagcagagccatggaaagagccctgagtggattggaggtattaatcctaacaatggtggtactacctacaaccagaaattcaagggcaaggccacattgactgtagacaagtcctccagcacagcctacatggagctccgcagcctgacatctgaggattctgcagtctattactgtgcgagacggattactacgatgatacctacggggggctttgactactggggccaaggcaccactctcacagtctcctca

SEQ ID NO: 38 - protein, 269 aa, Artificial Sequence
  synthetic protein
MEFGLSWLFLVAILKGVQCSRQIVLTQSPAIMSASPGEKVTMTCSASSSVSYMHWYQQKSGTSPKRWIYDTSKLASGVPARFSGSGSGTSYSLTISSMEAEDAATYYCLQWSSNPLTFGAGTKLEIKGGGGSGGGGSGGGGSGGGGSEVQLQQSGPELVKPGASVKISCKTSGYTFTEYTMHWVKQSHGKSPEWIGGINPNNGGTTYNQKFKGKATLTVDKSSSTAYMELRSLTSEDSAVYYCARRITTMIPTGGFDYWGQGTTLTVSS

SEQ ID NO: 39 - DNA, 318 bp, Artificial Sequence
  synthetic nucleic acid
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgcctgcagtggagtagtaacccgctcacgttcggtgctgggaccaagctggaaatcaaa

SEQ ID NO: 40 - protein, 106 aa, Artificial Sequence
  synthetic protein
QIVLTQSPAIMSASPGEKVTMTCSASSSVSYMHWYQQKSGTSPKRWIYDTSKLASGVPARFSGSGSGTSYSLTISSMEAEDAATYYCLQWSSNPLTFGAGTKLEIK

SEQ ID NO: 41 - protein, 10 aa, Artificial Sequence
  synthetic protein
SASSSVSYMH

SEQ ID NO: 42 - protein, 7 aa, Artificial Sequence
  synthetic protein
DTSKLAS

SEQ ID NO: 43 - protein, 9 aa, Artificial Sequence
  synthetic protein
LQWSSNPLT

SEQ ID NO: 44 - DNA, 366 bp, Artificial Sequence
  synthetic nucleic acid
gaggtccagctgcagcagtctggacctgagctggtgaagcctggggcttcagtgaagatatcctgcaagacttctggatacacattcactgaatacaccatgcactgggtgaagcagagccatggaaagagccctgagtggattggaggtattaatcctaacaatggtggtactacctacaaccagaaattcaagggcaaggccacattgactgtagacaagtcctccagcacagcctacatggagctccgcagcctgacatctgaggattctgcagtctattactgtgcgagacggattactacgatgatacctacggggggctttgactactggggccaaggcaccactctcacagtctcctca

SEQ ID NO: 45 - protein, 122 aa, Artificial Sequence
  synthetic protein
EVQLQQSGPELVKPGASVKISCKTSGYTFTEYTMHWVKQSHGKSPEWIGGINPNNGGTTYNQKFKGKATLTVDKSSSTAYMELRSLTSEDSAVYYCARRITTMIPTGGFDYWGQGTTLTVSS

SEQ ID NO: 46 - protein, 10 aa, Artificial Sequence
  synthetic protein
GYTFTEYTMH

SEQ ID NO: 47 - protein, 17 aa, Artificial Sequence
  synthetic protein
GINPNNGGTTYNQKFKG

SEQ ID NO: 48 - protein, 15 aa, Artificial Sequence
  synthetic protein
ARRITTMIPTGGFDY

SEQ ID NO: 49 - DNA, 789 bp, Artificial Sequence
  synthetic nucleic acid
atggagtttgggctgagctggctttttcttgtggctattttaaaaggtgtccagtgctctagagaaattttgctcacccagtctccagcaatcatagctgtatctcctggggagaaggtcaccataacctgcagtgccagctcaagtgtaaattacatgaactggtaccaacagaaaccaggatcctcccccaaaatatggatttttggtatgtccaacctggcttctggagttcctgctcgcttcagtggcagtgggtctgggacatctttctctttcacaatcaacagcatggaggctgaagatgttgccacttattactgtcaccaaaggagtagttacccatacacgttcggaggggggaccaagctggaaatcaaaggtggtggtggttctggtggtggtggttctggcggcggcggctccggtggtggtggatcccagatccagttggtgcagtctggacctgaattgaagaagcctggagagacagtcaggatctcctgcaaggcttctggatataccttcacaaactatggaatgaactgggtgaagcaggctccaggaaagggtttgaagtggatgggctggattaatacctacactggagagccgacatattctgatgtcttcaggggacggtttgccttctctttggaaacctctgccaacactgcctatttagagatcagaaaactcaaaaatgaggacatggctacgtatttctgtgtgagaaatgaatacgacgcaccttactggggccaagggactctggtcactgtctctgca

SEQ ID NO: 50 - protein, 263 aa, Artificial Sequence
  synthetic protein
MEFGLSWLFLVAILKGVQCSREILLTQSPAIIAVSPGEKVTITCSASSSVNYMNWYQQKPGSSPKIWIFGMSNLASGVPARFSGSGSGTSFSFTINSMEAEDVATYYCHQRSSYPYTFGGGTKLEIKGGGGSGGGGSGGGGSGGGGSQIQLVQSGPELKKPGETVRISCKASGYTFTNYGMNWVKQAPGKGLKWMGWINTYTGEPTYSDVFRGRFAFSLETSANTAYLEIRKLKNEDMATYFCVRNEYDAPYWGQGTLVTVSA

SEQ ID NO: 51 - DNA, 318 bp, Artificial Sequence
  synthetic nucleic acid
gaaattttgctcacccagtctccagcaatcatagctgtatctcctggggagaaggtcaccataacctgcagtgccagctcaagtgtaaattacatgaactggtaccaacagaaaccaggatcctcccccaaaatatggatttttggtatgtccaacctggcttctggagttcctgctcgcttcagtggcagtgggtctgggacatctttctctttcacaatcaacagcatggaggctgaagatgttgccacttattactgtcaccaaaggagtagttacccatacacgttcggaggggggaccaagctggaaatcaaa

SEQ ID NO: 52 - protein, 106 aa, Artificial Sequence
  synthetic protein
EILLTQSPAIIAVSPGEKVTITCSASSSVNYMNWYQQKPGSSPKIWIFGMSNLASGVPARFSGSGSGTSFSFTINSMEAEDVATYYCHQRSSYPYTFGGGTKLEIK

SEQ ID NO: 53 - protein, 10 aa, Artificial Sequence
  synthetic protein
SASSSVNYMN

SEQ ID NO: 54 - protein, 7 aa, Artificial Sequence
  synthetic protein
GMSNLAS

SEQ ID NO: 55 - protein, 9 aa, Artificial Sequence
  synthetic protein
HQRSSYPYT

SEQ ID NO: 56 - DNA, 348 bp, Artificial Sequence
  synthetic nucleic acid
cagatccagttggtgcagtctggacctgaattgaagaagcctggagagacagtcaggatctcctgcaaggcttctggatataccttcacaaactatggaatgaactgggtgaagcaggctccaggaaagggtttgaagtggatgggctggattaatacctacactggagagccgacatattctgatgtcttcaggggacggtttgccttctctttggaaacctctgccaacactgcctatttagagatcagaaaactcaaaaatgaggacatggctacgtatttctgtgtgagaaatgaatacgacgcaccttactggggccaagggactctggtcactgtctctgca

SEQ ID NO: 57 - protein, 116 aa, Artificial Sequence
  synthetic protein
QIQLVQSGPELKKPGETVRISCKASGYTFTNYGMNWVKQAPGKGLKWMGWINTYTGEPTYSDVFRGRFAFSLETSANTAYLEIRKLKNEDMATYFCVRNEYDAPYWGQGTLVTVSA

SEQ ID NO: 58 - protein, 10 aa, Artificial Sequence
  synthetic protein
GYTFTNYGMN

SEQ ID NO: 59 - protein, 17 aa, Artificial Sequence
  synthetic protein
WINTYTGEPTYSDVFRG

SEQ ID NO: 60 - protein, 9 aa, Artificial Sequence
  synthetic protein
VRNEYDAPY

SEQ ID NO: 61 - DNA, 816 bp, Artificial Sequence
  synthetic nucleic acid
atggagtttgggctgagctggctttttcttgtggctattttaaaaggtgtccagtgctctagaaacattgtgttggcccaatctccagcttctttggctgtgtctctagggcagagggccaccatatcctgcagagccagcgaaagtgttgataattatggctatagttttatgcactggtaccagcagaaaccaggacagtcacccaaactcctcatctatcttgcatccaacctagaatctggggtccctgccaggttcagtggcagtgggtctaggtcagacttcaccctcaccattgatcctgtggaggctgatgatgctgcaacctattactgtcagcaaaatagtgaagatccgtggacgttcggtggaggcaccaggctggaaatcaaaggtggtggtggttctggtggtggtggttctggcggcggcggctccggtggtggtggatcccaggttcaactccagcagtctggggctgtgctggtgaagcctggggcctcagtgaagatgtcctgcaaggcttctggctacacatttaccagttacaatttgcactggataaagcagacacctggacagggcctggaatggattggagttatttattcaggaaatagtgatacttcaaccaatcagaagttcaaaggcaaggccacattgactgaagacaaatccgccagtacagtctacatatatctcagcagcctgacatctgaagactctgcggtctattactgtgcaaggggggaatatggtaaccacgggggatttgcttactggggccaagggactctggtcactgtctctgca

SEQ ID NO: 62 - protein, 272 aa, Artificial Sequence
  synthetic protein
MEFGLSWLFLVAILKGVQCSRNIVLAQSPASLAVSLGQRATISCRASESVDNYGYSFMHWYQQKPGQSPKLLIYLASNLESGVPARFSGSGSRSDFTLTIDPVEADDAATYYCQQNSEDPWTFGGGTRLEIKGGGGSGGGGSGGGGSGGGGSQVQLQQSGAVLVKPGASVKMSCKASGYTFTSYNLHWIKQTPGQGLEWIGVIYSGNSDTSTNQKFKGKATLTEDKSASTVYIYLSSLTSEDSAVYYCARGEYGNHGGFAYWGQGTLVTVSA

SEQ ID NO: 63 - DNA, 333 bp, Artificial Sequence
  synthetic nucleic acid
aacattgtgttggcccaatctccagcttctttggctgtgtctctagggcagagggccaccatatcctgcagagccagcgaaagtgttgataattatggctatagttttatgcactggtaccagcagaaaccaggacagtcacccaaactcctcatctatcttgcatccaacctagaatctggggtccctgccaggttcagtggcagtgggtctaggtcagacttcaccctcaccattgatcctgtggaggctgatgatgctgcaacctattactgtcagcaaaatagtgaagatccgtggacgttcggtggaggcaccaggctggaaatcaaa

SEQ ID NO: 64 - protein, 111 aa, Artificial Sequence
  synthetic protein
NIVLAQSPASLAVSLGQRATISCRASESVDNYGYSFMHWYQQKPGQSPKLLIYLASNLESGVPARFSGSGSRSDFTLTIDPVEADDAATYYCQQNSEDPWTFGGGTRLEIK

SEQ ID NO: 65 - protein, 15 aa, Artificial Sequence
  synthetic protein
RASESVDNYGYSFMH

SEQ ID NO: 66 - protein, 7 aa, Artificial Sequence
  synthetic protein
LASNLES

SEQ ID NO: 67 - protein, 9 aa, Artificial Sequence
  synthetic protein
QQNSEDPWT

SEQ ID NO: 68 - DNA, 360 bp, Artificial Sequence
  synthetic nucleic acid
caggttcaactccagcagtctggggctgtgctggtgaagcctggggcctcagtgaagatgtcctgcaaggcttctggctacacatttaccagttacaatttgcactggataaagcagacacctggacagggcctggaatggattggagttatttattcaggaaatagtgatacttcaaccaatcagaagttcaaaggcaaggccacattgactgaagacaaatccgccagtacagtctacatatatctcagcagcctgacatctgaagactctgcggtctattactgtgcaaggggggaatatggtaaccacgggggatttgcttactggggccaagggactctggtcactgtctctgca

SEQ ID NO: 69 - protein, 120 aa, Artificial Sequence
  synthetic protein
QVQLQQSGAVLVKPGASVKMSCKASGYTFTSYNLHWIKQTPGQGLEWIGVIYSGNSDTSTNQKFKGKATLTEDKSASTVYIYLSSLTSEDSAVYYCARGEYGNHGGFAYWGQGTLVTVSA

SEQ ID NO: 70 - protein, 10 aa, Artificial Sequence
  synthetic protein
GYTFTSYNLH

SEQ ID NO: 71 - protein, 17 aa, Artificial Sequence
  synthetic protein
VIYSGNSDTSTNQKFKG

SEQ ID NO: 72 - protein, 13 aa, Artificial Sequence
  synthetic protein
ARGEYGNHGGFAY

SEQ ID NO: 73 - DNA, 804 bp, Artificial Sequence
  synthetic nucleic acid
atggagtttgggctgagctggctttttcttgtggctattttaaaaggtgtccagtgctctagagacattgtgatgacccagtctcaaaaattcatgtccacatcagtaggagacagggtcagcatcacctgcaaggccagtcagaatgttcgtactgctgtagcctggtatcaacagaaaccaggccagtctcctaaagcactgatttacttgccatccaaccggcacactggagtccctgatcgcttcacaggcagtggatctgggacagatttcactctcaccattagcaatgtgcaatctgaagacctggcagattatttctgtctgcaacattggaattatccattcacgttcggctcggggacaaagttggaaataaaaggtggtggtggttctggtggtggtggttctggcggcggcggctccggtggtggtggatcccaggtccagctgaagcagtctggagctgagctggtgaggcctggggcttcagtgaatctgtcctgcaagacttctggatacatcttcaccagctactggattcactgggtaaaacagaggtctggacagggccttgagtggattgcaaggatttatcctggagttgatagtatttattacaatgagaagttcaagggcaaggccacactgactgcagacaaatcctccagcactgcctacatgcagctcagcagcctgaaatctgaggactctgctgtctatttctgtgcaactatctcctatgattacgactattctatggactactggggtcaaggaacctcagtcaccgtctcctca

SEQ ID NO: 74 - protein, 268 aa, Artificial Sequence
  synthetic protein
MEFGLSWLFLVAILKGVQCSRDIVMTQSQKFMSTSVGDRVSITCKASQNVRTAVAWYQQKPGQSPKALIYLPSNRHTGVPDRFTGSGSGTDFTLTISNVQSEDLADYFCLQHWNYPFTFGSGTKLEIKGGGGSGGGGSGGGGSGGGGSQVQLKQSGAELVRPGASVNLSCKTSGYIFTSYWIHWVKQRSGQGLEWIARIYPGVDSIYYNEKFKGKATLTADKSSSTAYMQLSSLKSEDSAVYFCATISYDYDYSMDYWGQGTSVTVSS

SEQ ID NO: 75 - DNA, 321 bp, Artificial Sequence
  synthetic nucleic acid
gacattgtgatgacccagtctcaaaaattcatgtccacatcagtaggagacagggtcagcatcacctgcaaggccagtcagaatgttcgtactgctgtagcctggtatcaacagaaaccaggccagtctcctaaagcactgatttacttgccatccaaccggcacactggagtccctgatcgcttcacaggcagtggatctgggacagatttcactctcaccattagcaatgtgcaatctgaagacctggcagattatttctgtctgcaacattggaattatccattcacgttcggctcggggacaaagttggaaataaaa

SEQ ID NO: 76 - protein, 107 aa, Artificial Sequence
  synthetic protein
DIVMTQSQKFMSTSVGDRVSITCKASQNVRTAVAWYQQKPGQSPKALIYLPSNRHTGVPDRFTGSGSGTDFTLTISNVQSEDLADYFCLQHWNYPFTFGSGTKLEIK

SEQ ID NO: 77 - protein, 11 aa, Artificial Sequence
  synthetic protein
KASQNVRTAVA

SEQ ID NO: 78 - protein, 7 aa, Artificial Sequence
  synthetic protein
LPSNRHT

SEQ ID NO: 79 - protein, 9 aa, Artificial Sequence
  synthetic protein
LQHWNYPFT

SEQ ID NO: 80 - DNA, 359 bp, Artificial Sequence
  synthetic nucleic acid
aggtccagctgaagcagtctggagctgagctggtgaggcctggggcttcagtgaatctgtcctgcaagacttctggatacatcttcaccagctactggattcactgggtaaaacagaggtctggacagggccttgagtggattgcaaggatttatcctggagttgatagtatttattacaatgagaagttcaagggcaaggccacactgactgcagacaaatcctccagcactgcctacatgcagctcagcagcctgaaatctgaggactctgctgtctatttctgtgcaactatctcctatgattacgactattctatggactactggggtcaaggaacctcagtcaccgtctcctca

SEQ ID NO: 81 - protein, 120 aa, Artificial Sequence; identical to SEQ ID NO: 33
  synthetic protein

SEQ ID NO: 82 - protein, 10 aa, Artificial Sequence
  synthetic protein
GYIFTSYWIH

SEQ ID NO: 83 - protein, 17 aa, Artificial Sequence
  synthetic protein
RIYPGVDSIYYNEKFKG

SEQ ID NO: 84 - protein, 13 aa, Artificial Sequence
  synthetic protein
ATISYDYDYSMDY

SEQ ID NO: 85 - DNA, 813 bp, Artificial Sequence
  synthetic nucleic acid
atggagtttgggctgagctggctttttcttgtggctattttaaaaggtgtccagtgctctagagatattgtgatgacgcaggctgcattctccaatccagtcactcttggaacatcagcttccatctcctgcaggtctagtaagagtctcctccatagtaatggcatcacttatttctattggtatctgcagaagccaggccagtctcctcagctcctgatttatcagatgtccagccttgcctcaggagtcccagacaggttcagtagcagtgggtcaggaactgatttcacactgagaatcagcagagtggaggctgaggatgtgggtgtttattactgtgctcaaaatctagaacttccgtacacgttcggaggggggaccaagctggaaatcaaaggtggtggtggttctggtggtggtggttctggcggcggcggctccggtggtggtggatcccagatccagttggtgcagtctggacctgaggtgaggaggcctggggagacagtcaagatctcctgcaaggcttctggatatactttcacaaactttggaatgaactgggtgaaacaggctccaggaaaggctttacagtggatgggctggataaacacctacactggtgagccaacatatgctgatgacttcaagggccggtttgccttctctttggaatcctctgccagcactgcccatttgcagatcagctacctcaaagatgaggacacggctacatatttctgtgcgagatggtggcttcaatatgctttggacaactggggtcaaggaacctcagtcaccgtctcctca

SEQ ID NO: 86 - protein, 271 aa, Artificial Sequence
  synthetic protein
MEFGLSWLFLVAILKGVQCSRDIVMTQAAFSNPVTLGTSASISCRSSKSLLHSNGITYFYWYLQKPGQSPQLLIYQMSSLASGVPDRFSSSGSGTDFTLRISRVEAEDVGVYYCAQNLELPYTFGGGTKLEIKGGGGSGGGGSGGGGSGGGGSQIQLVQSGPEVRRPGETVKISCKASGYTFTNFGMNWVKQAPGKALQWMGWINTYTGEPTYADDFKGRFAFSLESSASTAHLQISYLKDEDTATYFCARWWLQYALDNWGQGTSVTVSS

SEQ ID NO: 87 - DNA, 336 bp, Artificial Sequence
  synthetic nucleic acid
gatattgtgatgacgcaggctgcattctccaatccagtcactcttggaacatcagcttccatctcctgcaggtctagtaagagtctcctccatagtaatggcatcacttatttctattggtatctgcagaagccaggccagtctcctcagctcctgatttatcagatgtccagccttgcctcaggagtcccagacaggttcagtagcagtgggtcaggaactgatttcacactgagaatcagcagagtggaggctgaggatgtgggtgtttattactgtgctcaaaatctagaacttccgtacacgttcggaggggggaccaagctggaaatcaaa

SEQ ID NO: 88 - protein, 112 aa, Artificial Sequence
  synthetic protein
DIVMTQAAFSNPVTLGTSASISCRSSKSLLHSNGITYFYWYLQKPGQSPQLLIYQMSSLASGVPDRFSSSGSGTDFTLRISRVEAEDVGVYYCAQNLELPYTFGGGTKLEIK

SEQ ID NO: 89 - protein, 16 aa, Artificial Sequence
  synthetic protein
RSSKSLLHSNGITYFY

SEQ ID NO: 90 - protein, 7 aa, Artificial Sequence
  synthetic protein
QMSSLAS

SEQ ID NO: 91 - protein, 9 aa, Artificial Sequence
  synthetic protein
AQNLELPYT

SEQ ID NO: 92 - DNA, 354 bp, Artificial Sequence
  synthetic nucleic acid
cagatccagttggtgcagtctggacctgaggtgaggaggcctggggagacagtcaagatctcctgcaaggcttctggatatactttcacaaactttggaatgaactgggtgaaacaggctccaggaaaggctttacagtggatgggctggataaacacctacactggtgagccaacatatgctgatgacttcaagggccggtttgccttctctttggaatcctctgccagcactgcccatttgcagatcagctacctcaaagatgaggacacggctacatatttctgtgcgagatggtggcttcaatatgctttggacaactggggtcaaggaacctcagtcaccgtctcctca

SEQ ID NO: 93 - protein, 118 aa, Artificial Sequence
  synthetic protein
QIQLVQSGPEVRRPGETVKISCKASGYTFTNFGMNWVKQAPGKALQWMGWINTYTGEPTYADDFKGRFAFSLESSASTAHLQISYLKDEDTATYFCARWWLQYALDNWGQGTSVTVSS

SEQ ID NO: 94 - protein, 10 aa, Artificial Sequence
  synthetic protein
GYTFTNFGMN

SEQ ID NO: 95 - protein, 17 aa, Artificial Sequence
  synthetic protein
WINTYTGEPTYADDFKG

SEQ ID NO: 96 - protein, 11 aa, Artificial Sequence
  synthetic protein
ARWWLQYALDN

SEQ ID NO: 97 - DNA, 816 bp, Artificial Sequence
  synthetic nucleic acid
atggagtttgggctgagctggctttttcttgtggctattttaaaaggtgtccagtgctctagaaacattgtgctgacccaatctccagcttctttggctgtgtctctagggcagagggccaccatatcctgcagagccagtgaaaatgttgataattatggcaatagttttatacactggtaccagcagaaaccaggacagccacccaaactcctcatctatcttgcatccaacctagaatctggggtccctgccaggttcagtggcagtgggtctaggtcagacttcaccctcaccattgatcctgtggaggctgatgatgctgcaacctattactgtcagcaaaataatgaggatccgtggacgttcggtggaggcaccaagctggaaatcaaaggtggtggtggttctggtggtggtggttctggcggcggcggctccggtggtggtggatcccaggtccaactgcagcagcctggggctgagctggtgaagcctggggcctcagtgaagatgtcctgcaaggcttctggctacatatttaccagttacaatttgcactgggtaaaacagacacctggacagggcctggaatggattggagttatttattcaggaaatagtgatacttccaacaatcagaagttcaaaggcaaggccacattgactgaagacaaatccgccagcacagcctacctacacctcagcagcctgacatctgaagactcagcggtctatttctgtgcaaggggggaatatggtaaccacggggggtttgcttactggggccaagggactctggtcactgtctctgca

SEQ ID NO: 98 - protein, 272 aa, Artificial Sequence
  synthetic protein
MEFGLSWLFLVAILKGVQCSRNIVLTQSPASLAVSLGQRATISCRASENVDNYGNSFIHWYQQKPGQPPKLLIYLASNLESGVPARFSGSGSRSDFTLTIDPVEADDAATYYCQQNNEDPWTFGGGTKLEIKGGGGSGGGGSGGGGSGGGGSQVQLQQPGAELVKPGASVKMSCKASGYIFTSYNLHWVKQTPGQGLEWIGVIYSGNSDTSNNQKFKGKATLTEDKSASTAYLHLSSLTSEDSAVYFCARGEYGNHGGFAYWGQGTLVTVSA

SEQ ID NO: 99 - DNA, 333 bp, Artificial Sequence
  synthetic nucleic acid
aacattgtgctgacccaatctccagcttctttggctgtgtctctagggcagagggccaccatatcctgcagagccagtgaaaatgttgataattatggcaatagttttatacactggtaccagcagaaaccaggacagccacccaaactcctcatctatcttgcatccaacctagaatctggggtccctgccaggttcagtggcagtgggtctaggtcagacttcaccctcaccattgatcctgtggaggctgatgatgctgcaacctattactgtcagcaaaataatgaggatccgtggacgttcggtggaggcaccaagctggaaatcaaa

SEQ ID NO: 100 - protein, 111 aa, Artificial Sequence
  synthetic protein
NIVLTQSPASLAVSLGQRATISCRASENVDNYGNSFIHWYQQKPGQPPKLLIYLASNLESGVPARFSGSGSRSDFTLTIDPVEADDAATYYCQQNNEDPWTFGGGTKLEIK

SEQ ID NO: 101 - protein, 15 aa, Artificial Sequence
  synthetic protein
RASENVDNYGNSFIH

SEQ ID NO: 102 - protein, 7 aa, Artificial Sequence
  synthetic protein
LASNLES

SEQ ID NO: 103 - protein, 9 aa, Artificial Sequence
  synthetic protein
QQNNEDPWT

SEQ ID NO: 104 - DNA, 360 bp, Artificial Sequence
  synthetic nucleic acid
caggtccaactgcagcagcctggggctgagctggtgaagcctggggcctcagtgaagatgtcctgcaaggcttctggctacatatttaccagttacaatttgcactgggtaaaacagacacctggacagggcctggaatggattggagttatttattcaggaaatagtgatacttccaacaatcagaagttcaaaggcaaggccacattgactgaagacaaatccgccagcacagcctacctacacctcagcagcctgacatctgaagactcagcggtctatttctgtgcaaggggggaatatggtaaccacggggggtttgcttactggggccaagggactctggtcactgtctctgca

SEQ ID NO: 105 - protein, 120 aa, Artificial Sequence
  synthetic protein
QVQLQQPGAELVKPGASVKMSCKASGYIFTSYNLHWVKQTPGQGLEWIGVIYSGNSDTSNNQKFKGKATLTEDKSASTAYLHLSSLTSEDSAVYFCARGEYGNHGGFAYWGQGTLVTVSA

SEQ ID NO: 106 - protein, 10 aa, Artificial Sequence
  synthetic protein
GYIFTSYNLH

SEQ ID NO: 107 - protein, 17 aa, Artificial Sequence
  synthetic protein
VIYSGNSDTSNNQKFKG

SEQ ID NO: 108 - protein, 13 aa, Artificial Sequence
  synthetic protein
ARGEYGNHGGFAY

SEQ ID NO: 109 - DNA, 795 bp, Artificial Sequence
  synthetic nucleic acid
atggagtttgggctgagctggctttttcttgtggctattttaaaaggtgtccagtgctctagagacatccagatgactcagtctccagcctccctaactgtatttgtgggggaaactgtcaccatcacatgtcgagcaagtgagaatatttacagtaatttagcgtggtatcagcagaaacagggaaaatctcctcagctcctggtctatactgcaacaaatttaatagatggtgtgccatcaaggttcagtggcagtgggtccggcacacagtattccctcaagatcaacagcctgcagtctgaagatttcgggagttattactgtcaacatttttggggtactccggtcacgttcggtgctgggaccaagctggaaatcaaaggtggtggtggttctggtggtggtggttctggcggcggcggctccggtggtggtggatcccaggtccaactgcagcagcctggggcagaactggtgaagcctggggcctcagtgaggatgtcctgcagggcttctggctacacttttaccagttacaatttgcactgggtaaaacagacacctggacagggcctggaatggattggggctattcatccaggagataattatacttcctacaatcagattttcaacgacaaggccacattgactgcagacaaatcctccagcacagcctacatgcagctcagtagcctgacatctgaggactctgcggtctatttctgtgcaaggaattactacggtagtcttgattactggggccaaggcaccactctcacagtctcctca

SEQ ID NO: 110 - protein, 265 aa, Artificial Sequence
  synthetic protein
MEFGLSWLFLVAILKGVQCSRDIQMTQSPASLTVFVGETVTITCRASENIYSNLAWYQQKQGKSPQLLVYTATNLIDGVPSRFSGSGSGTQYSLKINSLQSEDFGSYYCQHFWGTPVTFGAGTKLEIKGGGGSGGGGSGGGGSGGGGSQVQLQQPGAELVKPGASVRMSCRASGYTFTSYNLHWVKQTPGQGLEWIGAIHPGDNYTSYNQIFNDKATLTADKSSSTAYMQLSSLTSEDSAVYFCARNYYGSLDYWGQGTTLTVSS

SEQ ID NO: 111 - DNA, 321 bp, Artificial Sequence
  synthetic nucleic acid
gacatccagatgactcagtctccagcctccctaactgtatttgtgggggaaactgtcaccatcacatgtcgagcaagtgagaatatttacagtaatttagcgtggtatcagcagaaacagggaaaatctcctcagctcctggtctatactgcaacaaatttaatagatggtgtgccatcaaggttcagtggcagtgggtccggcacacagtattccctcaagatcaacagcctgcagtctgaagatttcgggagttattactgtcaacatttttggggtactccggtcacgttcggtgctgggaccaagctggaaatcaaa

SEQ ID NO: 112 - protein, 107 aa, Artificial Sequence
  synthetic protein
DIQMTQSPASLTVFVGETVTITCRASENIYSNLAWYQQKQGKSPQLLVYTATNLIDGVPSRFSGSGSGTQYSLKINSLQSEDFGSYYCQHFWGTPVTFGAGTKLEIK

SEQ ID NO: 113 - protein, 11 aa, Artificial Sequence
  synthetic protein
RASENIYSNLA

SEQ ID NO: 114 - protein, 6 aa, Artificial Sequence
  synthetic protein
TATNLI

SEQ ID NO: 115 - protein, 9 aa, Artificial Sequence
  synthetic protein
QHFWGTPVT

SEQ ID NO: 116 - DNA, 351 bp, Artificial Sequence
  synthetic nucleic acid
caggtccaactgcagcagcctggggcagaactggtgaagcctggggcctcagtgaggatgtcctgcagggcttctggctacacttttaccagttacaatttgcactgggtaaaacagacacctggacagggcctggaatggattggggctattcatccaggagataattatacttcctacaatcagattttcaacgacaaggccacattgactgcagacaaatcctccagcacagcctacatgcagctcagtagcctgacatctgaggactctgcggtctatttctgtgcaaggaattactacggtagtcttgattactggggccaaggcaccactctcacagtctcctca

SEQ ID NO: 117 - protein, 117 aa, Artificial Sequence
  synthetic protein
QVQLQQPGAELVKPGASVRMSCRASGYTFTSYNLHWVKQTPGQGLEWIGAIHPGDNYTSYNQIFNDKATLTADKSSSTAYMQLSSLTSEDSAVYFCARNYYGSLDYWGQGTTLTVSS

SEQ ID NO: 118 - protein, 10 aa, Artificial Sequence
  synthetic protein
GYTFTSYNLH

SEQ ID NO: 119 - protein, 17 aa, Artificial Sequence
  synthetic protein
AIHPGDNYTSYNQIFND

SEQ ID NO: 120 - protein, 10 aa, Artificial Sequence
  synthetic protein
ARNYYGSLDY

SEQ ID NO: 121 - DNA, 135 bp, Homo sapiens
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 122 - protein, 45 aa, Homo sapiens
TTTPAPRPPTPAPTIASQPLSLRPEACRPAAGGAVHTRGLDFACD

SEQ ID NO: 123 - DNA, 72 bp, Homo sapiens
atctacatctgggcgcccttggccgggacttgtggggtccttctcctgtcactggttatcaccctttactgc

SEQ ID NO: 124 - protein, 24 aa, Homo sapiens
IYIWAPLAGTCGVLLLSLVITLYC

SEQ ID NO: 125 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 126 - protein, 41 aa, Homo sapiens
RSKRSRLLHSDYMNMTPRRPGPTRKHYQPYAPPRDFAAYRS

SEQ ID NO: 127 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 128 - protein, 42 aa, Homo sapiens
KRGRKKLLYIFKQPFMRPVQTTQEEDGCSCRFPEEEEGGCEL

SEQ ID NO: 129 - DNA, 339 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 130 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0055426 A1
(43) Pub. Date: Feb. 23, 2023

(54) CHIMERIC ANTIGEN RECEPTOR AND IMMUNE EFFECTOR CELL EXPRESSING CHIMERIC ANTIGEN RECEPTOR

(71) Applicant: Boyuan Runsheng Pharma (Hangzhou) Co., Ltd., Hangzhou (CN)

(72) Inventors: Chuang Sun, Hangzhou (CN); Xin-Hua Feng, Hangzhou (CN); Bin Zhao, Hangzhou (CN)

(21) Appl. No.: 17/635,226

(22) PCT Filed: Aug. 6, 2020
(86) PCT No.: PCT/CN2020/107484

§ 371 Date: Feb. 14, 2022

(30) Foreign Application Priority Data
Aug. 14, 2019 (CN) 201910750137.1

(51) Int. Cl. C07K 16/28; C12N 15/86; A61P 35/00

(52) U.S. Cl. CPC C07K 16/2827; C12N 15/86; A61P 35/00; C07K 2317/565; C07K 2317/622; C07K 2317/53; C07K 2317/24

(57) ABSTRACT

Provided are a chimeric antigen receptor capable of targeting B7-H3, a nucleic acid molecule encoding the chimeric antigen receptor, a nucleic acid construct comprising the nucleic acid molecule, an immune effector cell expressing the chimeric antigen receptor, and use thereof. The chimeric antigen receptor comprises an anti-B7-H3 binding domain, a hinge region, a transmembrane domain and a signal transduction domain. Further provided are a composition and a method for diagnosing, treating or preventing tumors that express B7-H3.

This application claims the priority of Chinese patent application CN 201910750137.1 filed with the China National Intellectual Property Administration on Aug. 14, 2019, the entirety of which is incorporated herein by reference.

### TECHNICAL FIELD

The present disclosure relates to the field of biological medicines, and particularly relates to a B7-H3-targeted chimeric antigen receptor, immune effector cells expressing the B7-H3-targeted chimeric antigen receptor, and use thereof.

### BACKGROUND ART

Cancer is one of the main diseases threatening human health. Common cancer therapies include radiotherapy, chemotherapy, targeted therapy, etc., and currently, immunotherapy is gradually becoming a mainstream therapy. Chimeric antigen receptor-mediated cell therapy is a type of immunotherapy, and CD19-targeted chimeric antigen receptor T-cell therapy has made outstanding achievements in the treatment of B-cell-associated tumors, and has cured some patients (whose tumors do not recur in 5 years). Therefore, these achievements have prompted FDA's approval of the CD19-targeted chimeric antigen receptor T-cell therapy, which promotes the rapid development of this therapy in the field of blood cancer. However, due to the lack of specific targets, the heterogeneity of solid tumor (different tumor cells have different phenotypes), tumor microenvironment, etc., the chimeric antigen receptor-mediated cell therapy didn't perform well for the treatment of solid tumors, and therefore, developing chimeric antigen receptors for new targets is particularly critical to solving this problem.

B7-H3 is a membrane protein belonging to the B7 immunomodulatory factor family, which is present in two isoforms of 21g-B7-H3 and 41g-B7-H3 in the human body, but only in one isoform of 21g-B7-H3 in mice. Some literature reported that B7-H3 could reduce the level of type I interferon released by T cells, and could also inhibit the killing function of natural killer cells, and therefore, B7-H3 is regarded as an immunosuppressive factor. Other reports also confirmed that B7-H3 can inhibit diseases such as graft-versus-host reaction, heart transplant rejection, airway inflammation, and experimental autoimmune encephalomyelitis, etc. However, in certain literature, B7-H3 is described as a T-cell costimulatory factor in the in vitro and in vivo autoimmune models.

B7-H3 is expressed at very low level in normal human tissues but is usually overexpressed (ranges from 74%-94%) in tumors. In additions, studies have found that B7-H3 is also highly expressed in tumor-associated blood vessels and tumor stromal fibroblasts. The high expression of B7-H3 is closely related with a low number of tumor-infiltrating lymphocytes, the rapid progression of cancer, and a poor clinical outcome, and is common in cancers such as glioma, pancreatic cancer, prostate cancer, ovarian cancer, lung cancer, and kidney cancer, etc. Because B7-H3 is highly expressed in a variety of solid tumors, it can be a good target for solid tumors. Meanwhile, B7-H3 is expressed on the cell surface, so that chimeric antigen receptor-mediated cell therapy can be used to target B7-H3 to kill tumor cells without damaging normal cells.

### SUMMARY OF THE INVENTION

In view of the above, the present disclosure is directed to provide a B7-H3 protein-targeted chimeric antigen receptor (CAR), immune effector cells expressing the CAR, and use thereof in cancer diagnosis, treatment, and prevention.

Specifically, the present disclosure proposes the following technical solutions:

In one aspect, the present disclosure provides a B7-H3-targeted chimeric antigen receptor (CAR), characterized by including: an anti-B7-H3 binding domain, a hinge region, a transmembrane domain, and a signal transduction domain.

In one aspect, in the CAR of the present disclosure, the anti-B7-H3 binding domain comprises an anti-B7-H3 single-chain antibody or antigen-binding portion, and the antibody or antigen-binding portion comprises a light-chain CDR selected from amino acid sequences of SEQ ID NO: 5-7, 17-19, 29-31, 41-43, 53-55, 65-67, 77-79, 89-91, 101-103, 113-115 or any variant thereof; and/or, comprises a heavy-chain CDR selected from amino acid sequences of SEQ ID NO: 10-12, 22-24, 34-36, 46-48, 58-60, 70-72, 82-84, 94-96, 106-108, 118-120 or any variant thereof.

In the CAR according to any one of the preceding aspects, the anti-B7-H3 binding domain comprises an anti-B7-H3 antibody or antigen-binding portion, and the antibody or antigen-binding portion comprises a light-chain CDR1 selected from amino acid sequences of SEQ ID NO: 5, 17, 29, 41, 53, 65, 77, 89, 101, 113 or any variant thereof, a light-chain CDR2 selected from amino acid sequences of SEQ ID NO: 6, 18, 30, 42, 54, 66, 78, 90, 102, 114 or any variant thereof, and a light-chain CDR3 selected from amino acid sequences of SEQ ID NO: 7, 19, 31, 43, 55, 67, 79, 91, 103, 115 or any variant thereof; and/or, comprises a heavy-chain CDR1 selected from amino acid sequences of SEQ ID NO: 10, 22, 34, 46, 58, 70, 82, 94, 106, 118 or any variant thereof, a heavy-chain CDR2 selected from amino acid sequences of SEQ ID NO: 11, 23, 35, 47, 59, 71, 83, 95, 107, 119 or any variant thereof, and a heavy-chain CDR3 selected from amino acid sequences of SEQ ID NO: 12, 24, 36, 48, 60, 72, 84, 96, 108, 120 or any variant thereof.

In the CAR according to any one of the preceding aspects, the anti-B7-H3 binding domain comprises an anti-B7-H3 antibody or antigen-binding portion, and the antibody or antigen-binding portion comprises a light-chain sequence selected from amino acid sequences of SEQ ID NO: 4, 16, 28, 40, 52, 64, 76, 88, 100, 112 or any variant thereof; and/or, comprises a heavy-chain sequence selected from amino acid sequences of SEQ ID NO: 9, 21, 33, 45, 57, 69, 81, 93, 105, 117 or any variant thereof.

In the CAR according to any one of the preceding aspects, the anti-B7-H3 binding domain comprises an anti-B7-H3 antibody (scFv) domain; preferably, the anti-B7-H3 single-chain antibody has an amino acid sequence selected from amino acid sequences of SEQ ID NO: 2, 14, 26, 38, 50, 62, 74, 86, 98, 110 or any variant thereof.

In the CAR according to any one of the preceding aspects, the transmembrane domain is selected from one or more of transmembrane domains of α, β, ζ chains of TCR, CD3γ, CD3δ, CD3ε, CD3ζ, CD4, CD5, CD8α, CD9, CD16, CD22, CD27, CD28, CD33, CD37, CD45, CD64, CD80, CD86, CD134, 4-1BB, CD152, CD154, PD-1; preferably, the transmembrane domain is selected from one or more of transmembrane domains of CD8α, CD4, CD45, PD-1, CD154, CD28; preferably, the transmembrane domain is selected from one or more of transmembrane domains of CD8a or CD28; more preferably, the transmembrane domain has an amino acid sequence selected from amino acid sequences of SEQ ID NO: 124 or any variant thereof; the hinge region is selected from one or more of an extracellular hinge region of CD8, a hinge region of IgG1 Fc CH2CH3, a hinge region of IgD, an extracellular hinge region of CD28, a hinge region of IgG4 Fc CH2CH3, and an extracellular hinge region of CD4; preferably, the hinge region is a hinge region of CD8α; more preferably, the hinge region has an amino acid sequence selected from amino acid sequences of SEQ ID NO: 122 or any variant thereof.

In the CAR according to any one of the preceding aspects, the signal transduction domain is selected from one or more of intracellular signaling regions of TCRζ, FcRγ, FcRβ, CD3γ, CD3δ, CD3ε, CD5, CD22, CD79a, CD79b, CD278(ICOS), CD66d, CD3ζ; preferably, the signal transduction domain is selected from an intracellular signaling region of CD3ζ; more preferably, the signal transduction domain has an amino acid sequence selected from amino acid sequences of SEQ ID NO: 130 or any variant thereof; preferably, the signal transduction domain further comprises one or more costimulatory domains; preferably, the costimulatory domain is selected from one or more of intracellular signaling regions of CARD11, CD2, CD7, CD27, CD28, CD30, CD40, CD54, CD83, OX40, 4-1BB, CD134, CD150, CD152, CD223, CD270, PD-L2, PD-L1, CD278, DAP10, LAT, NKD2C, SLP76, TRIM, FcεRIγ, MyD88, 4-1BB; preferably, the costimulatory domain is selected from intracellular signaling regions of 4-1BB, CD134, CD28, and OX40; preferably, the costimulatory domain is selected from one or more of intracellular signaling regions of 4-1BB or CD28; more preferably, the costimulatory domain has an amino acid sequence selected from amino acid sequence of SEQ ID NO: 126, 128 or any variant thereof.

In one aspect, the present disclosure provides an isolated nucleic acid molecule, comprising a polynucleotide sequence encoding the CAR according to any one of the preceding aspects; preferably, the nucleic acid molecule has a nucleotide sequence selected from nucleotide sequences of SEQ ID NO: 1, 13, 25, 37, 49, 61, 73, 85, 97, 109 or any variant thereof.

In one aspect, the present disclosure provides a nucleic acid construct, comprising the nucleic acid molecule according to any one of the preceding aspects; preferably, the nucleic acid construct is a viral vector; more preferably, the viral vector is one or more of a retroviral vector, a lentiviral vector, an adenoviral vector, an adeno-associated viral vector.

In one aspect, the present disclosure provides a virus, comprising the nucleic acid molecule according to any one of the preceding aspects, or comprising the nucleic acid construct according to any one of the preceding aspects; preferably, the virus is one or more of a retrovirus, a lentivirus, an adenovirus, an adeno-associated virus.

The present disclosure also provides use of the CAR according to any one of the preceding aspects, the nucleic acid molecule according to any one of the preceding aspects, the nucleic acid construct according to any one of the preceding aspects, or the virus according to any one of the preceding aspects in preparation of genetically modified immune cells targeting tumor cells expressing B7-H3.

In one aspect, the present disclosure provides an isolated host cell, expressing the CAR according to any one of the preceding aspects, or comprising the nucleic acid molecule according to any one of the preceding aspects, or comprising the nucleic acid construct according to any one of the preceding aspects, or comprising the virus according to any one of the preceding aspects; preferably, the host cell is a mammalian cell; more preferably, the host cell is one or more of a T cell, an NK cell or cell line, a γδT cell, an NKT cell, a macrophage or cell line, a PG13 cell line; more preferably, the host cell is a T cell; the most preferably, the host cell is a primarily cultured T cell.

The host cell according to any one of the preceding aspects also expresses other effector molecules that include, but are not limited to, a cytokine, another CAR, a chemokine receptor, an siRNA reducing the expression of PD-1 or a protein blocking PD-L1, a TCR, or a safety switch;

preferably, the cytokine is selected from one or more of TNF-α, TNF-β, VEGF, TPO, NGF-β, PDGF, TGF-α, TGF-β, IGF-I, IGF-II, EPO, M-CSF, IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, an interferon, an angiostatin, a thrombospondin, or an endostatin;

preferably, the chemokine receptor is selected from one or more of CCR2, CCR5, CXCR2, CXCR4;

preferably, the safety switch is selected from one or more of HSVTK, VZVTK, iCaspase-9, iCaspase-1, iCaspase-8, truncated EGFR, RQR8.

In one aspect, the present disclosure provides a pharmaceutical composition, comprising one or more of the CAR according to any one of the preceding aspects, the nucleic acid molecule according to any one of the preceding aspects, the nucleic acid construct according to any one of the preceding aspects, the virus according to any one of the preceding aspects, the host cell according to any one of the preceding aspects, and a pharmaceutically acceptable carrier.

The present disclosure also provides use of the CAR according to any one of the preceding aspects, the nucleic acid molecule according to any one of the preceding aspects, the nucleic acid construct according to any one of the preceding aspects, the virus according to any one of the preceding aspect, the host cell according to any one of the preceding aspects, or the pharmaceutical composition in preparation of a drug; preferably, the drug is used to diagnose, treat or prevent one or more of cancer, an inflammatory disease, an autoimmune disease; preferably, the drug is used to diagnose, treat or prevent a tumor expressing B7-H3; more preferably, the drug is used to diagnose, treat or prevent one or more of brain cancer, pancreatic cancer, ovarian cancer, kidney cancer, bladder cancer, skin cancer, stomach cancer, intestinal cancer, head and neck cancer, thyroid cancer, prostate cancer, lung cancer, and Kaposi's sarcoma; more preferably, the brain cancer is selected from one or more of glioblastoma, astrocytoma, meningioma, oligodendroglioma, neuroglioma.

The present disclosure achieves the following beneficial effects:

Mouse models of brain glioma have verified that the B7-H3-targeted chimeric antigen receptor-T cells prepared in the present disclosure could remove tumors in most of the mice, and could prevent tumors from recurring within a certain period; and meanwhile, the survival time of the mice was significantly prolonged, which indicates that the B7-H3-targeted chimeric antigen receptor-T cells of the present disclosure can kill tumor cells in vivo, and can be used as a potential therapy for cancer.

### BRIEF DESCRIPTION OF THE DRAWINGS

FIG. 1: the expression of B7-H3 in different tumor cell lines.

FIG. 2: the expression of B7-H3 in brain glioma samples.

FIG. 3: the basic structure of a B7-H3-targeted chimeric antigen receptor, and in the figure, B7-H3.28 CAR represents a chimeric antigen receptor whose costimulatory domain is CD28, and B7-H3.BB CAR represents a chimeric antigen receptor whose costimulatory domain is 4-1BB.

FIG. 4: FIG. 4A: the expression of the humanized antibody 15C11-derived B7-H3-targeted chimeric antigen receptor according to the present disclosure on T cells (from an individual healthy donor); and FIG. 4B: the expression of the humanized antibody 15C11-derived B7-H3-targeted chimeric antigen receptor according to the present disclosure on T cells (from multiple healthy donors).

FIG. 5: the killing effects of humanized antibody 15C11-derived B7-H3-targeted chimeric antigen receptor-T cells according to the present disclosure on different types of tumor cells in the co-culture systems in vitro.

FIG. 6: FIG. 6A: comparing with the other chimeric antigen receptors (CAR1 and CAR2) targeting the same antigen, the affinity of the humanized antibody 15C11-derived B7-H3-targeted chimeric antigen receptor to the antigen according to the present disclosure; and FIG. 6B: comparing with the other chimeric antigen receptors (CAR1 and CAR2) targeting the same antigen when killing tumor cells, the levels of cytokines released by the humanized antibody 15C11-derived B7-H3-targeted chimeric antigen receptor CAR-T cells according to the present disclosure.

FIG. 7: the killing effect of the humanized antibody 15C11-derived B7-H3-targeted chimeric antigen receptor-T cells according to the present disclosure on brain glioma in vivo.

FIG. 8: the increased survival rates of mouse models of brain glioma that are treated with the humanized antibody 15C11-derived B7-H3-targeted chimeric antigen receptor-T cells according to the present disclosure.

FIG. 9: FIG. 9A shows the killing effect of the humanized antibody 15C11-derived B7-H3-targeted chimeric antigen receptor-T cells according to the present disclosure on patient-derived glioma neurospheres in vitro (bright field); and FIG. 9B shows the killing effect of the humanized antibody 15C11-derived B7-H3-targeted chimeric antigen receptor-T cells according to the present disclosure on patient-derived glioma neurospheres in vitro (flow cytometry).

FIG. 10: the humanized antibody 15C11-derived B7-H3-targeted chimeric antigen receptor-T cells according to the present disclosure significantly prolong the survival time of patient-derived glioma neurosphere xenograft mouse models.

FIG. 11: the killing effects of 9 kinds of other murine-derived B7-H3-targeted antigen receptor-T cells on tumor cells in vitro.

FIG. 12: the cytokines released by the 9 kinds of murine-derived B7-H3-targeted antigen receptor-T cells when killing tumors.

### DETAILED DESCRIPTION OF THE INVENTION

### I. Definitions

Unless other specified, the scientific and technical terms used herein have the same meaning as understood by those skilled in the art. Furthermore, terms related to protein and nucleic acid chemistry, molecular biology, cell and tissue culture, microbiology, and immunology, and experimental steps used herein are the widely used terms and conventional steps in the corresponding art. Meanwhile, in order to provide a better understanding of the present disclosure, the relevant terms will be defined and described below.

It should be understood that the terms are used for the purpose of describing specific embodiments, and are not intended to limit the present disclosure.

As used herein, unless otherwise specified, the term “about”, when referring to a measurable value such as an amount and a period of time, is meant to encompass variations of ±10%, more preferably ±5%, even more preferably ±1%, and still more preferably ±0.1% from the given value, as long as such variations are suitable for practicing the disclosed methods.

As used herein, the term “activation” refers to a state of T cells that are sufficiently stimulated to induce detectable cell proliferation. Activation can also be associated with induced cytokine production and detectable effector function. The terms “activated T cells” and the like refer to T cells undergoing cell division.

As used herein, the term “antibody” refers to an immunoglobulin molecule that specifically binds to an antigen. An antibody may be a natural or recombinant intact immunoglobulin or may be an immune response portion of an intact immunoglobulin. An antibody is generally a tetramer of immunoglobulin molecules. The antibody of the present disclosure can exist in a variety of forms which include, but are not limited to, a polyclonal antibody, a monoclonal antibody, Fv, Fab, F(ab)2, etc., and a single-chain antibody and a humanized antibody (Harlow, et al., 1999, Using Antibodies: A Laboratory Manual, Cold Spring Harbor Laboratory Press, NY; Harlow, et al., 1989, Antibodies: A Laboratory Manual, Cold Spring Harbor, N.Y.; Houston, et al., 1988, Proc. Natl. Acad. Sci., USA 85: 5879-5883; and Bird, et al., 1988, Science 242: 423-426).

As used herein, an “antibody fragment” or “antigen-binding fragment” of an antibody refers to any part of a full-length antibody, which is shorter than the full-length antibody, but at least comprises a partial variable region (e.g., one or more CDRs and/or one or more antigen-binding sites) binding to an antigen of the antibody and thus reserves binding specificity and at least partial specific binding ability of the full-length antibody. Therefore, an antigen-binding fragment refers to an antibody fragment comprising an antigen-binding portion that binds to the same antigen as an antibody from which the antibody fragment is derived. An antibody fragment includes an antibody derivative produced by enzymatic treatment of a full-length antibody, and a derivative produced by synthesis such as a derivative produced by recombination. An antibody comprises antibody fragments.

Embodiments of the antibody fragment include, but are not limited to, Fab, Fab′, F(ab′)2, single-chain Fv (scFv), Fv, dsFv, double-antibody, Fd and Fd′ fragments, and other fragments including modified fragments (referring to, for example, Methods in Molecular Biology, Vol 207: Recombinant Antibodies for Cancer Therapy Methods and Protocols (2003); Chapter 1; p 3-25, Kipriyanov). The fragments may comprise multiple chains that are linked together, for example, via disulfide bonds and/or via peptide linkers. An antibody fragment generally comprises at least or about 50 amino acids, and typically comprises at least or about 200 amino acids. An antigen-binding fragment includes any antibody fragment that is inserted into an antibody framework (for example, through replacement of a corresponding region) to obtain an antibody that immunospecifically binds (that is, showing Ka of at least or at least about 10′-10‘ M-’) to an antigen. A “functional fragment” or “anti-B7-H3 antibody analogue” is a fragment or analogue that can prevent or substantially reduce the ability of a receptor to bind to a ligand or initiate signal transduction. As used herein, a functional fragment generally has the same meaning as an “antibody fragment”, and for an antibody, it may refer to a fragment that can prevent or substantially reduce the ability of a receptor to bind to a ligand or initiate signal transduction, such as Fv, Fab, and F(ab′)2. An “Fv” fragment is composed of a dimer (VH-VL dimer) formed by a variable domain of one heavy chain and a variable domain of one light chain by mean of non-covalent binding. In this configuration, 3 CDRs of each variable domain interact to determine target-binding sites on the surface of the VH-VL dimer, which is the same as an intact antibody. The 6 CDRs together confer the target-binding specificity of the intact antibody. However, even a single variable domain (or a half of an Fv that comprises only 3 target-specific CDRs) can still have the ability to recognize and bind to a target.

As used herein, a “monoclonal antibody” refers to a population of identical antibodies, which means that each single antibody molecule in the monoclonal antibody population is identical to other antibody molecules. This property is in contrast to that of a population of polyclonal antibodies, which comprises a variety of antibodies having different sequences. A monoclonal antibody can be prepared by a number of well-known methods (Smith, et al., (2004) J. Clin. Pathol. 57, 912-917; and Nelson, et al., J Clin Pathol (2000), 53, 111-117). For example, a monoclonal antibody can be prepared by immortalizing B cells, for example, by fusing with myeloma cells to generate a hybridoma cell line or by infecting B cells with a virus such as EBV. Recombinant technologies can also be used to prepare an antibody from a clonal population of host cells in vitro by transforming the host cells with a plasmid carrying an artificial sequence of nucleotides encoding the antibody.

As used herein, a “heavy chain” of an antibody refers to the larger chain in the two types of polypeptide chains that exist in all antibody molecules in their autogenetic conformations.

As used herein, a “light chain” of an antibody refers to the smaller chain in the two types of polypeptide chains that exist in all antibody molecules in their autogenetic conformations, and κ and λ light chains refer to the two major isoforms of a light chain of an antibody.

As used herein, the term “scFv” refers to a fusion protein that comprises at least one antibody fragment comprising a variable region of a light chain and at least one antibody fragment comprising a variable region of a heavy chain, the variable regions of the light chain and the heavy chain are linked via a short flexible polypeptide linker, and can be expressed as a single-chain polypeptide, the scFv reserves the specificity of an intact antibody from which it is derived. Unless otherwise specified, as used herein, scFv can comprise the VL and VH variable regions in any order (e.g., relative to the N-terminal end and the C-terminal end of a polypeptide), and scFv can comprise VL-linker-VH or can comprise VH-linker-VL.

As used herein, the term “gene synthesis” refers to the production by using a recombinant DNA technology or the acquisition by using a synthetic DNA or amino acid sequence technology available and well known in the art.

As used herein, the term “antigen” or “Ag” is defined as a molecule that elicits an immune response, which may involve antibody production, or activation of specific immunocompetent cells, or both. Those skilled in the art will understand that any macromolecule, actually including all proteins or peptides, can be used as an antigen. In addition, an antigen can be derived from recombinant or genomic DNA. Those skilled in the art will understand that any DNA, comprising a nucleotide sequence or partial nucleotide sequence encoding a protein that elicits an immune response, encodes an “antigen” as used herein. In addition, those skilled in the art will understand that an antigen is not necessarily encoded separately by a full-length nucleotide sequence of a gene. It is readily apparent that the present invention includes, but is not limited to, use of more than one partial nucleotide sequences of a gene, and such nucleotide sequences are arranged in different combinations to elicit a desired immune response. In addition, those skilled in the art will understand that an antigen is not necessarily encoded by a “gene”. It is readily apparent that an antigen can be produced, synthesized or derived from a biological sample. The biological sample may include, but is not limited to, a tissue sample, a tumor sample, a cell, and a biological fluid.

As used herein, the term “anti-tumor effect” refers to a biological effect that can be clearly indicated by reduction in tumor volume, reduction in the number of tumor cells, reduction in the number of metastases, increase in life expectancy, or improvements of various physiological symptoms associated with cancer. An “anti-tumor effect” can also be clearly indicated by the ability of the peptide, the polynucleotide, the cell or the antibody of the present disclosure to prevent a tumor from forming at the first position.

As used herein, the term “cancer” is defined as a disease characterized by rapid and uncontrolled growth of aberrant cells. Cancer cells can spread locally or spread to other parts of the body via the blood flow and the lymphatic system. Examples of various cancers include, but are not limited to, brain cancer (e.g., astrocytoma, meningioma, oligodendroglioma, and neuroglioma), pancreatic cancer, ovarian cancer, kidney cancer, bladder cancer, skin cancer, stomach cancer, intestinal cancer, head and neck cancer, thyroid cancer, prostate cancer, lung cancer, Kaposi's sarcoma, etc.

As used herein, examples of the term “inflammatory disease” include, but are not limited to, asthma, encephalitis, inflammatory bowel disease, chronic obstructive pulmonary disease (COPD), allergy, septic shock, pulmonary fibrosis, undifferentiated spondyloarthropathy, undifferentiated osteoarthritis, arthritis, inflammatory osteolysis, and chronic inflammation caused by chronic viral infection and bacterial infection.

As used herein, examples of the term “autoimmune disease” include, but are not limited to, alopecia areata, ankylosing spondylitis, antiphospholipid syndrome, autoimmune Addison's disease, adrenal autoimmune disease, autoimmune hemolytic anemia, autoimmune hepatitis, autoimmune oophoritis and orchitis, autoimmune thrombocytopenia, Behget's disease, bullous pemphigoid, cardiomyopathy, celiac sprue, dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus erythematosus, idiopathic mixed cryoglobulinemia, fibromyalgia-fibromyositis, glomerulonephritis, Graves' disease, Guillain-Barré syndrome, Hashimoto's thyroiditis, idiopathic pulmonary fibrosis, idiopathic thrombocytopenic purpura (ITP), IgA neuropathy, juvenile arthritis, oral lichen planus, lupus erythematosus, Ménière's disease, mixed connective tissue disease, multiple sclerosis, Type I or immune-mediated diabetes, myasthenia gravis, pemphigus vulgaris, pernicious anemia, polyarteritis nodosa, polychondritis, polyglandular syndrome, polymyalgia rheumatica, polymyositis and dermatomyositis, primary agammaglobulinemia, primary biliary cholangitis, psoriasis, psoriatic arthritis, Raynaud's disease, Reiter's syndrome, rheumatoid arthritis, sarcoidosis, scleroderma, Sjögren's syndrome, catalepsy, systemic lupus erythematosus, lupus erythematosus, polyarteritis, temporal arteritis/giant cell arteritis, ulcerative colitis, uveitis, and vasculitis such as dermatitis herpetiformis vasculitis, vitiligo, and Wegener's granulomatosis. Certain autoimmune diseases are associated with an inflammatory state. Therefore, there is overlap between symptoms considered to be autoimmune diseases and inflammatory diseases. Therefore, certain autoimmune diseases can also be called inflammatory diseases.

As used herein, the term “costimulatory ligand” includes molecules on antigen-presenting cells (e.g., APCs, dendritic cells, and B cells) that specifically bind to associated costimulatory molecules on T cells to provide the primary signal by, for example, binding a TCR/CD3 complex to a peptide-loaded MHC molecule and provide a signal that mediates a T-cell response, which includes, but is not limited to, proliferation, activation, differentiation, etc. A costimulatory ligand may include, but is not limited to, CD7, B7-1 (CD80), B7-2 (CD86), PD-L1, PD-L2, 4-1BBL, OX40L, an inducible costimulatory ligand (ICOS-L), an intercellular adhesion molecule (ICAM), CD30L, CD40, CD70, CD83, HLA-Q MICA, MICB, HVEM, a lymphotoxin-β receptor, 3/TR6, ILT3, ILT4, HVEM, an agonist or antibody that binds to a Toll ligand receptor, and a ligand that specifically binds to B7-H3. A costimulatory ligand also includes, particularly, an antibody that specifically binds to costimulatory molecules existing on T cells, such as, but not limited to, antibodies that specifically bind to CD27, CD28, 4-1BB, OX40, CD30, CD40, PD-1, ICOS, a lymphocyte function-associated antigen-1 (LFA-1), CD2, CD7, LIGHT, NKG2C, B7-H3, and CD83.

As used herein, the term “costimulatory molecule” refers to an associated binding partner on a T cell that specifically binds to a costimulatory ligand to mediate a costimulatory response of the T cell, such as, but not limited to, proliferation, and the costimulatory molecule includes, but is not limited to, an MHC-class I molecule, and BTLA and Toll ligand receptors.

As used herein, the term “costimulatory signal” refers to a signal that binds to the primary signal for example, through TCR/CD3 ligation, to elicit T-cell proliferation and/or up-regulation or down-regulation of key molecules.

“Encoding” refers to the inherent property of specific sequences of nucleotides in a polynucleotide, such as a gene, cDNA, and mRNA, to serve as a template for synthesis of other polymers and macromolecules in the biological process, the polymers and macromolecules have any one of a defined sequence of a nucleotide (i.e., rRNA, tRNA, and mRNA) or a defined sequence of an amino acid and biological properties resulting therefrom. Therefore, the transcription and translation of mRNA corresponding to a gene produces proteins in cells or other biological systems, which means that the gene encodes proteins. Both of the coding strand whose nucleotide sequence is identical to the mRNA sequence, which is usually provided in a sequence listing, and the non-coding strand, which serves as a template for transcribing a gene or cDNA, can be referred to as encoding proteins or other products of that gene or cDNA.

As used herein, the term “endogenous” refers to any substance derived from an organism, cell, tissue or system, or produced within an organism, cell, tissue or system.

As used herein, the term “exogenous” refers to any substance introduced into an organism, cell, tissue or system, or produced outside an organism, cell, tissue or system. “Expression” is defined as the transcription and/or translation of a specific nucleotide sequence driven by its promoter.

As used herein, the term “nucleic acid construct” refers to a vector comprising a recombinant polynucleotide that comprises an expression control sequence operably linked to a nucleotide sequence to be expressed. The nucleic acid construct comprises sufficient cis-acting elements for expressing; and other elements for expressing can be provided by host cells or provided in an expression system in vitro. The nucleic acid construct includes those known in the art, such as a cosmid, a plasmid (e.g., exposed to or comprised in a liposome), and a virus (e.g., a lentivirus, a retrovirus, an adenovirus, and an adeno-associated virus) that are incorporated into a recombinant polynucleotide. “Homology” refers to the sequence similarity or sequence identity of two peptides or two nucleic acid molecules.

When sites in two compared sequences are occupied by the same base or amino acid monomer subunit, for example, if a site in each of two DNA molecules is occupied by an adenine, the molecules are homologous at that site. Percentage of the homology of two sequences is calculated by a function of the number of matched or homologous sites co-owned by two sequences/the number of compared sitesx 100. For example, two sequences are 60% homologous if 6 out of 10 sites in the two sequences are matched or homologous. For example, two DNA sequences ATTGCC and TATGGC are 50% homologous. Usually, two sequences are compared to obtain the maximum homology.

As used herein, the term “immunoglobulin” or “Ig” is defined as a class of proteins that achieve an antibody effect.

An antibody expressed by B cells are sometimes known as BCR (B-cell receptor) or an antigen receptor. It includes 5 members of IgA, IgQ IgM, IgD, and IgE in the class of proteins. IgA is a primary antibody existing in body secretions such as saliva, tears, breast milk, a gastrointestinal secretion, and mucinous secretions in the respiratory tract and the urogenital tract. IgG is the most common circulating antibody. IgM is a predominant immunoglobulin produced during the primary immune response of most subjects. It is the most effective immunoglobulin in agglutination, complement fixation, and other antibody responses, which plays a very important role in combating bacteria and viruses. IgD is an immunoglobulin without the known antibody function, which can be used as an antigen receptor. IgE is an immunoglobulin that mediates immediate hypersensitivity after exposure to an allergen by eliciting mediator release from mast cells and basophils.

“Isolation” refers to transformation or emigration from the natural state. For example, a nucleic acid or peptide naturally existing in a living animal is not “isolated”, but the same nucleic acid or peptide that is partially or completely “isolated” from the coexisting substance in its natural state is “isolated”. An isolated nucleic acid or protein can exist in a substantially purified form, or, for example, can exist in a non-natural environment such as host cells.

Unless otherwise specified, a “polynucleotide sequence encoding an amino acid sequence” includes all nucleotide sequences that are degenerate versions of each other and encode the same amino acid sequence. A phrase nucleotide sequence encoding a protein or RNA may also include introns, to the extent that the nucleotide sequence encoding the protein may include (one or more) introns in some versions.

As used herein, the term “operable linkage” refers to a functional linkage between a regulatory sequence and a heterologous nucleic acid sequence, which produces the expression of the latter. For example, if a first nucleic acid sequence is in a functional relationship with a second nucleic acid sequence, the first nucleic acid and the second nucleic acid sequence are operably linked. For example, if a promoter affects the transcription or expression of a coding sequence, the promoter is operably linked to the coding sequence. Usually, operably linked DNA sequences are contiguous, and two protein-coding regions are necessarily be linked in the same reading frame.

The term “overexpressed” tumor antigen or “overexpression” of a tumor antigen is intended to indicate the abnormal expression level of the tumor antigen in cells from a disease region such as a solid tumor in a specific tissue or organ of a patient, relative to the expression level of normal cells from a tissue or an organ. Patients with solid tumors or haematological malignancies characterized by tumor antigen overexpression can be determined by standard assays known in the art.

“Parenteral” administration of an immunogenic composition includes, such as, subcutaneous (s.c), intravenous (i.v.), intramuscular (i.m.) or intrasternal injection, or injection technologies.

As used herein, the terms “patient”, “subject”, “individual” and the like are interchangeable, and refer to any animal or cells thereof that, whether in vitro or in situ, comply with the methods described herein. In some nonrestrictive implementation modes, a patient, subject or individual refers to human.

As used herein, the term “polynucleotide” is defined as a nucleotide chain. In addition, a nucleic acid is a polymer of nucleotides. Therefore, as used herein, the nucleic acid and the polynucleotide are interchangeable. Those skilled in the art have the general knowledge that a nucleic acid is a polynucleotide that can be hydrolyzed into monomeric “nucleotides”. A monomeric nucleotide can be hydrolyzed into nucleosides. As used herein, the polynucleotide includes, but is not limited to, all nucleic acid sequences obtained by any available method in the art, and the methods include, but are not limited to, a recombination method, that is, nucleic acid sequences are cloned from a recombination library or a genome of a cell by a common cloning technology, PCR™ or the like, and a synthesis method.

As used herein, the terms “peptide”, “polypeptide”, and “protein” are interchangeable, and refer to a compound composed of amino acid residues covalently linked via peptide bonds. A protein or peptide necessarily comprises at least two amino acids, and there is no limit to the maximum number of amino acids that can include a sequence of a protein or peptide. A polypeptide includes any peptide or protein including two or more amino acids linked to each other via peptide bonds. As used herein, this term refers to a short chain that is usually known as, such as, a peptide, an oligopeptide, and an oligomer in the art; and a relative long chain that is usually known as a protein in the art and has many types. A “polypeptide” includes, such as, a biologically active fragment, substantially homologous polypeptides, oligopeptide, a homodimer, a heterodimer, a variant of a polypeptide, a modified polypeptide, a derivative, an analogue, a fusion protein, etc. A polypeptide includes a natural peptide, a recombinant peptide, a synthesized peptide, or a combination thereof.

As used herein, the term “safety switch” refers to an engineered protein that is designed to prevent potential toxicity of cell therapy or prevent adverse effects by other methods. In some cases, the expression of a safety switch protein is conditionally controlled to address safety issues of an engineered cell to be transplanted that have permanently incorporated a gene encoding the safety switch protein into a genome of the cell. Such conditional control may be variable and may include control through micromolecule-mediated post-translational activation and tissue specificity and/or on-time transcriptional control. The safety switch can mediate induction of apoptosis, inhibition of protein synthesis, DNA replication, growth retardation, transcription, and post-transcription gene control, and/or antibody-mediated depletion. In some cases, the safety switch protein is activated by an exogenous molecule (e.g., a prodrug), and triggers cells to be treated to undergo apoptosis and/or death upon activation. Embodiments of the safety switch protein include, but are not limited to, HSVTK, VZVTK, iCaspase-9, iCaspase-1, iCaspase-8, truncated EGFR, RQR8, etc. In such strategy, a prodrug administrated in the case of adverse event is activated by a suicide gene product to kill transduced cells.

As used herein, the term “promoter” is defined as a DNA sequence that is required by initiation of the specific transcription of a polynucleotide sequence, is recognized by a synthesis mechanism of cells or guides the synthesis mechanism.

As used herein, the term “promoter/regulatory sequence” refers to a nucleic acid sequence required by the expression of a gene product that can be operably linked to the promoter/regulatory sequence. In some examples, this sequence may be a core promoter sequence, and in other examples, this sequence may also comprise an enhancer sequence and other regulatory elements that are required by the expression of a gene product. A promoter/regulatory sequence may be, such as, a sequence expressing a gene product in a tissue specificity manner.

A “constitutive” promoter is a nucleotide sequence that, when operably linked to a polynucleotide encoding or specifying a gene product, enable a gene product to be produced in a cell under most or all cellular physiological conditions.

A “inducible” promoter is a nucleotide sequence that, when operably linked to a polynucleotide encoding or specifying a gene product, enable a gene product to be produced in a cell substantially only when an inducer corresponding to the promoter exists in the cell.

A “tissue-specific” promoter is a nucleotide sequence that, when operably linked to a polynucleotide encoding a gene or specified by a gene, enable a gene product to be produced in a cell substantially only when the cell is corresponding to the tissue type of the promoter.

As used herein, the “specific binding” or “immunospecific binding” of an antibody or antigen-binding fragment thereof is interchangeable, and refers to the ability of the antibody or antigen-binding fragment to form one or more non-covalent bonds with an alloantigen through non-covalent interactions between the antibody and antibody-binding sites of the antigen. The antigen may be an isolated antigen or an antigen existing in a tumor cell.

Usually, an antibody that immunospecifically binds (or specifically binds) to an antigen binds the antigen according to an affinity constant Ka of about 1×10⁷ M⁻¹ or 1×10 M⁻¹ or above (or according to a dissociation constant (Kd) of 1×10⁻⁷ M or 1×10⁻⁸ M or below). An affinity constant can be determined by a standard kinetic method for antibody responses, such as immunoassay, surface plasmon resonance (SPR) (Rich and Myszka (2000) Curr. Opin. Biotechnol 11: 54; and Englebienne (1998) Analyst. 123: 1599), isothermal titration calorimetry (ITC), and other kinetic interaction measurement methods known in the art (referring to, such as, Paul, ed., Fundamental Immunology, 2nd ed., Raven Press, New York, pages 332-336 (1989); and referring to the U.S. patent application 7,229,619 which discloses exemplary SPR and ITC for calculating the binding affinity of an antibody). Instruments and methods for detecting and monitoring binding rates in real time are known and commercially available in the market (referring to BiaCore 2000, Biacore AB, Upsala, Sweden and GE Healthcare Life Sciences; Malmqvist (2000) Biochem. Soc. Trans. 27: 335).

As used herein, a “substantially purified” cell refer to a cell that substantially does not comprise other types of cells.

A substantially purified cell also refers to a cell that has been isolated from other types of cells with which it is normally associated in its naturally occurring state. In some examples, a substantially purified cell mass refers to a homogeneous cell mass. In other examples, this term simply refers to a cell that has been isolated from other types of cells with which it is normally associated in its native state. In some embodiments, cells are cultured in vitro. In other embodiments, cells are not cultured in vitro.

As used herein, the term “therapeutic” refers to treatment and/or prevention. A therapeutic effect is achieved through disease inhibition, alleviation or eradication.

As used herein, the term “therapeutically effective amount” refers to the mount of an object compound which will elicit a biological or medical response of a tissue, a system or an object sought by a researcher, a veterinarian, a medical doctor or other clinicians. The term “therapeutically effective amount” includes the following amount of a compound: when administrated, it is sufficient to prevent the development of one or more of signs or symptoms of a disorder or disease to be treated, or alleviate one or more of signs or symptoms of the disorder or disease to be treated. The therapeutically effective amount is adjusted according to compounds, diseases and their severities, and age, weight, etc. of subjects to be treated.

As used herein, the term “treatment” of a disease refers to reduction in the frequency or severity of at least one sign or symptom of a disease or disorder experienced by a subject.

As used herein, the term “transfection” or “transformation” or “transduction” refers to the process by which exogenous nucleic acids are transferred or introduced into host cells. “Transfected” or “transformed” or “transduced” cells are cells that have been transfected, transformed or transduced with exogenous nucleic acids.

These cells include primary object cells and their progeny.

As used herein, the term “vector” is a substance composition that comprises isolated nucleic acids and can be used to transfer the isolated nucleic acids into cells. Many vectors are known in the art, which include, but are not limited to, a linear polynucleotide, a polynucleotide associated with an ionic or amphiphilic compound, a plasmid, and a virus. Therefore, the term “vector” includes an autonomously replicating plasmid or virus. This term should also be construed to include a non-plasmid and non-viral compound that facilitates transfer of nucleic acids into cells, such as a poly-lysine compound and a liposome. Examples of the viral vector include, but not are limited to, an adenoviral vector, an adeno-associated viral vector, a retroviral vector, etc.

### II. Specific Embodiments

In one aspect, the present disclosure provides a B7-H3-targeted chimeric antigen receptor, and immune effector cells expressing the B7-H3-targeted chimeric antigen receptor; and specifically, the immune effector cells expressing the B7-H3-targeted chimeric antigen receptor are T cells expressing the B7-H3-targeted chimeric antigen receptor.

In one aspect, the B7-H3-targeted chimeric antigen receptor (CAR) of the present disclosure comprises an anti-B7-H3 binding domain, a hinge region, a transmembrane domain, and a signal transduction domain.

In the CAR according to any one of the preceding aspects, the anti-B7-H3 binding domain comprises an anti-B7-H3 antibody or antigen-binding portion, and the antibody or antigen-binding portion comprises a light-chain CDR selected from amino acid sequences of SEQ ID NO: 5-7, 17-19, 29-31, 41-43, 53-55, 65-67, 77-79, 89-91, 101-103, 113-115 or any variant thereof; and/or, comprises a heavy-chain CDR selected from amino acid sequences of SEQ ID NO: 10-12, 22-24, 34-36, 46-48, 58-60, 70-72, 82-84, 94-96, 106-108, 118-120 or any variant thereof.

In the CAR according to any one of the preceding aspects, the anti-B7-H3 binding domain comprises an anti-B7-H3 antibody or antigen-binding portion, and the antibody or antigen-binding portion comprises a light-chain CDR1 selected from amino acid sequences of SEQ ID NO: 5, 17, 29, 41, 53, 65, 77, 89, 101, 113 or any variant thereof, a light-chain CDR2 selected from amino acid sequences of SEQ ID NO: 6, 18, 30, 42, 54, 66, 78, 90, 102, 114 or any variant thereof, and a light-chain CDR3 selected from amino acid sequences of SEQ ID NO: 7, 19, 31, 43, 55, 67, 79, 91, 103, 115 or any variant thereof; and/or, comprises a heavy-chain CDR1 selected from amino acid sequences of SEQ ID NO: 10, 22, 34, 46, 58, 70, 82, 94, 106, 118 or any variant thereof, a heavy-chain CDR2 selected from amino acid sequences of SEQ ID NO: 11, 23, 35, 47, 59, 71, 83, 95, 107, 119 or any variant thereof, and a heavy-chain CDR3 selected from amino acid sequences of SEQ ID NO: 12, 24, 36, 48, 60, 72, 84, 96, 108, 120 or any variant thereof.

In the CAR according to any one of the preceding aspects, the anti-B7-H3 binding domain comprises an anti-B7-H3 antibody or antigen-binding portion, and the antibody or antigen-binding portion comprises a light chain sequence selected from amino acid sequences of SEQ ID NO: 4, 16, 28, 40, 52, 64, 76, 88, 100, 112 or any variant thereof; and/or, comprises a heavy chain sequence selected from amino acid sequences of SEQ ID NO: 9, 21, 33, 45, 57, 69, 81, 93, 105, 117 or any variant thereof.

In the CAR according to any one of the preceding aspects, the anti-B7-H3 binding domain comprises an anti-B7-H3 single-chain antibody (scFv).

In the CAR according to any one of the preceding aspects, the anti-B7-H3 single-chain antibody is derived from a monoclonal antibody produced against a B7-H3 protein fragment.

In the CAR according to any one of the preceding aspects, the anti-B7-H3 single-chain antibody has an amino acid sequence selected from amino acid sequences of SEQ ID NO: 2, 14, 26, 38, 50, 62, 74, 86, 98, 110 or any variant thereof.

In one aspect, the anti-B7-H3 binding domain and the anti-B7-H3 single-chain antibody part according to any one of the preceding aspects have at least more than 60%, 65%, 70%, 75%, 80%, 85%, 90%, 95%, 96%, 97%, 98%, 99% or preceding sequence identity.

In the CAR according to any one of the preceding aspects, the transmembrane domain is selected from one or more transmembrane domains of α, β, ζ chains of TCR, CD3γ, CD3δ, CD3ε, CD3ζ, CD4, CD5, CD8α, CD9, CD16, CD22, CD27, CD28, CD33, CD37, CD45, CD64, CD80, CD86, CD134, 4-1BB, CD152, CD154, PD-1; preferably, the transmembrane domain is selected from one or more transmembrane domains of CD8α, CD4, CD45, PD-1, CD154, CD28; more preferably, the transmembrane domain is selected from one or more transmembrane domains of CD8a or CD28; more preferably, the transmembrane domain has an amino acid sequence selected from amino acid sequence of SEQ ID NO: 124 or any variant thereof.

In the CAR according to any one of the preceding aspects, the hinge region is selected from one or more of an extracellular hinge region of CD8, a hinge region of IgG1 Fc CH2CH3, a hinge region of IgD, an extracellular hinge region of CD28, a hinge region of IgG4 Fc CH2CH3, and an extracellular hinge region of CD4; preferably, the hinge region is a hinge region of CD8α; more preferably, the hinge region has an amino acid sequence selected from amino acid sequences of SEQ ID NO: 122 or any variant thereof.

In the CAR according to any one of the preceding aspects, the signal transduction domain is selected from one or more intracellular signaling regions of TCRζ, FcRγ, FcRβ, CD3γ, CD3δ, CD3ε, CD5, CD22, CD79a, CD79b, CD278 (ICOS), CD66d, CD3ζ; preferably, the signal transduction domain is selected from an intracellular signaling region of CD3ζ; more preferably, the signal transduction domain has an amino acid sequence selected from amino acid sequences of SEQ ID NO: 130 or any variant thereof.

In the CAR according to any one of the preceding aspects, the signal transduction domain further comprises one or more costimulatory domains; preferably, the costimulatory domain is selected from one or more intracellular signaling regions of CARD11, CD2, CD7, CD27, CD28, CD30, CD40, CD54, CD83, OX40, 4-1BB, CD134, CD150, CD152, CD223, CD270, PD-L2, PD-L1, CD278, DAP10, LAT, NKD2C, SLP76, TRIM, FcεFRIγ, MyD88, 41BBL; more preferably, the costimulatory domain is selected from intracellular signaling regions of 4-1BB, CD134, CD28, OX40; more preferably, the costimulatory domain is selected from one or more intracellular signaling regions of 4-1BB or CD28; more preferably, the costimulatory domain has an amino acid sequence selected from amino acid sequences of SEQ ID NO: 126, 128 or any variant thereof.

In another aspect, the present disclosure also provides a B7-H3-targeted single-chain antibody (scFv) domain; specifically, the scFv is derived from a monoclonal antibody produced against a B7-H3 protein fragment; more specifically, the scFv has an amino acid sequence selected from amino acid sequences of SEQ ID NO: 2, 14, 26, 38, 50, 62, 74, 86, 98, 110 or any variant thereof.

In one aspect, an encoding gene of the B7-H3-targeted scFv comprises a nucleotide sequence selected from nucleotide sequences of SEQ ID NO: 1, 13, 25, 37, 49, 61, 73, 85, 97, 109 or any variant thereof.

In one aspect, the present disclosure provides a nucleic acid molecule encoding the CAR or scFv according to any one of the preceding aspects; preferably, the nucleic acid molecule encoding the scFv has a nucleotide sequence selected from nucleotide sequences of SEQ ID NO: 1, 13, 25, 37, 49, 61, 73, 85, 97, 109 or a nucleic acid molecule with at least more than 60%, 65%, 70%, 75%, 80%, 85%, 90%, 95%, 96%, 97%, 98%, 99% or preceding sequence identity.

In one aspect, the present disclosure provides a nucleic acid construct, comprising the nucleic acid molecule according to any one of the preceding aspects; preferably, the nucleic acid construct is a viral vector; more preferably, the viral vector is selected from one or more of a retroviral vector, a lentiviral vector, an adenoviral vector.

In one aspect, the present disclosure provides a virus, comprising the nucleic acid molecule according to any one of the preceding aspects, or comprising the nucleic acid construct according to any one of the preceding aspects; and preferably, the virus is selected from one or more of a retrovirus, a lentivirus, an adenovirus, an adeno-associated virus.

In one aspect, the present disclosure provides an isolated host cell, expressing the CAR according to any one of the preceding aspects, or comprising the nucleic acid construct according to any one of the preceding aspects, or comprising the virus according to any one of the preceding aspects; preferably, the host cell is a mammalian cell; more preferably, the host cell is a T cell, an NK cell or cell line, a γδT cell, an NKT cell, a macrophage or cell line, a PG13 cell line; more preferably, the host cell is a T cell; the most preferably, the host cell is a primarily cultured T cell.

In one aspect, the host cell also expresses other effector molecules that include, but are not limited to, a cytokine, another CAR, a chemokine receptor, an siRNA reducing the expression of PD-1 or a protein blocking PD-L1, a TCR or a safety switch; preferably, the cytokine is selected from one or more of TNF-α, TNF-β, VEGF, TPO, NGF-β, PDGF, TGF-α, TGF-β, IGF-I, IGF-II, EPO, M-CSF, IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, an interferon, an angiostatin, a thrombospondin, an endostatin; more preferably, the chemokine receptor is selected from one or more of CCR2, CCR5, CXCR2, CXCR4; preferably, the safety switch is selected from one or more of HSVTK, VZVTK, iCaspase-9, iCaspase-1, iCaspase-8, truncated EGFR, RQR8.

In one aspect, the present disclosure provides an immune effector cell expressing the B7-H3-targeted chimeric antigen receptor; specifically, the present disclosure provides a T cell expressing the B7-H3-targeted chimeric antigen receptor (CAR-T); more specifically, the T cell is an activated T cell; more specifically, the activation of the T cell is completed by stimulation with an CD3 and/or CD28 antibodies.

In one aspect, the present disclosure provides a pharmaceutical composition, comprising one or more of the CAR, the nucleic acid molecule, the nucleic acid construct, the virus, the cell according to any one of the preceding aspects, and a pharmaceutically acceptable carrier.

It should be understood that the therapeutic agent of the present disclosure will be administrated together with an appropriate pharmaceutically acceptable carrier, an excipient, and other reagents that are incorporated into the formulation to provide improved transfer, delivery, tolerability, etc. A large number of suitable formulations can be found in the pharmacopoeia known to all pharmaceutical chemists: Remington's Pharmaceutical Science (15th edition, Mack Publishing Company, Easton, Pa. (1975)), especially Chapter 87 of Blaug, Seymour therein. These formulations include, for example, powders, pastes, ointments, gels, waxes, oils, lipids, lipid-comprising (cation or anion) carriers (e.g., Lipofectin™), DNA conjugates, anhydrous slurries, oil-in-water and water-in-oil emulsions, emulsion polyethylene glycols (polyethylene glycols of various molecular weights), semi-solid gels, and semi-solid mixtures comprising polyethylene glycols. Any above mixture can be applicable to the treatment or therapy of the present disclosure under the condition that active ingredients in the formulation will be not inactivated by the formulation and the formulation is physiologically compatible and tolerant during administration.

As used herein, the term “pharmaceutically acceptable carrier” is intended to include any and all solvents, dispersion media, coatings, antibacterial agents and antifungal agents, isotonic agents and absorption delaying agents, etc. that are compatible with pharmaceutical administration. Appropriate pharmaceutically acceptable carriers are described in the latest edition of Remington's Pharmaceutical Sciences, which is a standard reference in the art and is incorporated herein by reference. Preferred examples of these carriers or diluents include, but are not limited to, water, saline, a Ringer's solution, a glucose solution, and 1-10% human serum albumin. Liposomes and non-aqueous carriers, such as fixed oils, can also be used. The use of these media and reagents for pharmaceutically active substances is well known in the art. In addition to any conventional media or reagents that are incompatible with the antibody, its use in compositions is contemplated.

Further, the pharmaceutical composition of the present disclosure can be used to treat inflammatory factor-associated diseases such as cancer, inflammatory diseases, autoimmune diseases, etc.

In one aspect, the CAR, the nucleic acid molecule, the nucleic acid construct, the virus, the cell, and the pharmaceutical composition according to any one of the preceding aspects are used to prepare a drug for diagnosing, treating or preventing cancer.

In one embodiment, the drug can be used as a therapeutic agent. Such reagent is usually used to diagnose, treat, relieve and/or prevent diseases or pathology associated with abnormal B7-H3 expression, activity and/or signal transduction of a subject. A subject, such as a human patient suffering from (or at risk of or developing) a disease or disorder associated with abnormal B7-H3 expression, activity and/or signal transduction, such as cancer or other neoplastic disorders, can be identified by a standard method and applied with a therapeutic regimen. An antibody or antibody fragment that specifically binds to a target antigen (e.g., B7-H3) is used as the chimeric antigen receptor (CAR) of the present disclosure, preferably, an antibody with high specificity and high affinity to its target antigen (e.g., B7-H3) is prepared into the CAR of the present invention, and preferably, further, the prepared CAR is used to genetically modify immune cells (e.g., T cells). The expression, activity and/or signal transduction function of a target (e.g., B7-H3) can be eliminated, inhibited or hindered by administrating the CAR and/or genetically modified immune cells of the present disclosure. The binding of a target (e.g., B7-H3) to its naturally bound endogenous ligand can be eliminated or inhibited or hindered by administrating the CAR and/or genetically modified immune cells of the present disclosure. For example, the B7-H3 expression, activity and/or signal transduction can be regulated, blocked, inhibited, reduced, antagonized, neutralized, or hindered in other manners by administrating the CAR and/or genetically modified immune cells of the present disclosure. In some embodiments, in order to treat a disease or disorder associated with abnormal B7-H3 expression, an anti-B7-H3 antibody heavy-chain and light-chain CDR antibody or antibody fragment can be prepared into the CAR and/or genetically modified immune cells of the present disclosure, and then the CAR and/or genetically modified immune cells are administered to a subject. In one embodiment, the disease or disorder associated with abnormal B7-H3 expression may be cancer.

In order to clearly and concisely describe the present disclosure, features are described herein as part of the same or separate embodiments. However, it will be understood that the scope of the present disclosure may include embodiments comprising combinations of all or some of the described features.

### EXAMPLES

### Example 1: Detection of the Expression of B7-H3 in Tumors

In order to determine the expression levels of the antigen B7-H3 in tumors, we first selected different tumor cell lines for detection. Meanwhile, to further verify the expression of B7-H3 in primary tumors, we detected B7-H3 expression levels in sections of brain glioma tissues and normal tissues of multiple patients.

Method: tumor cells were collected, washed with 1×PBS three times, stained with an B7-H3 antibody 15C11 on ices for 30 min, washed with 1×PBS, added with an APC-conjugated goat-anti-mouse secondary antibody, incubated at room temperature for 20 min, and washed with 1×PBS, and the expression of B7-H3 on tumor cells was determined by using flow cytometry.

Immunohistochemistry staining: the expression of B7-H3 in primary tumors was detected by immunohistochemistry staining the sections of brain glioma tissues and normal tissues of multiple patients with the 15C11 antibodies.

The paraffin sections were deparaffinized with dimethylbenzene and rewatered with ethyl alcohol, and antigens were repaired with sodium citrate. Then, the sections were blocked in serums for 1 h. The sections were stained with the B7-H3 antibodies 15C11 at 4° C. overnight, and then incubated with horseradish peroxidase-conjugated goat-anti-mouse secondary antibodies for 30 min. The sections were washed, developed with DAB substrates, and then stained with hematoxylin. After being stained, the sections were observed and photographed under a microscope.

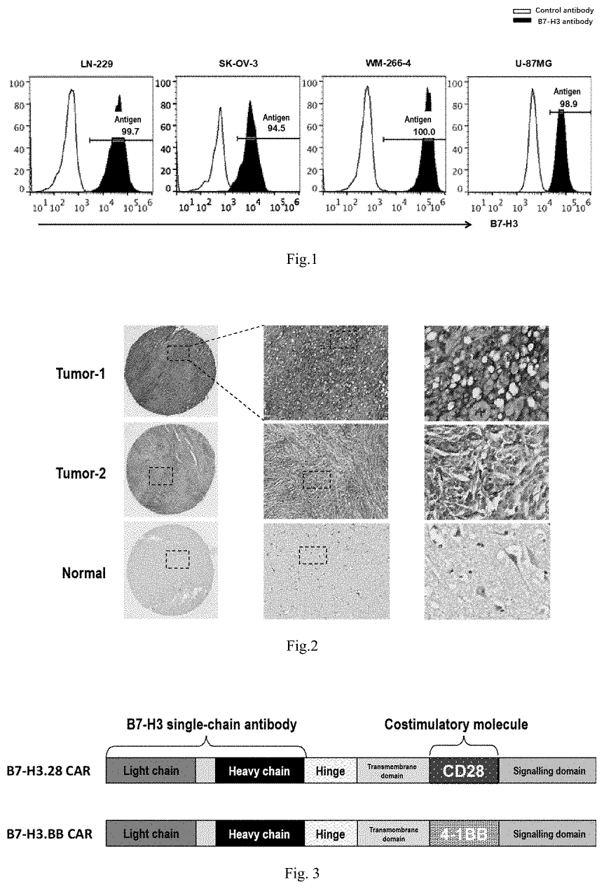

Results: As shown in FIG. 1, B7-H3 is highly expressed in the detected cell lines of several types of tumors, such as skin cancer (WM-266-4), ovarian cancer (SK-OV-3) and glioma (LN-229, U-87MG). FIG. 2 showed that in the glioma samples of all patients, different levels of B7-H3 expression is detected, and in normal tissues adjacent to the tumor and normal brain tissues, B7-H3 is only expressed in a few cells at very low levels. It indicates that B7-H3 could be a specific target for solid tumor therapy.

### Example 2: Cell Culture

Cell lines used in the present disclosure, including 293T cells, were cultured in IMDM media comprising 10% FBS, 1% GlutaMAX, and 1% two types of antibiotics.

Cell culture media for WM-266-4, SK-OV-3, LN-229, LN-18, U-87MG were RPMI640 or DMEM with 10% FBS.

A retrovirus encoding GFP was prepared by transiently transfecting 293T cells with a three-plasmid system (GFS-GFP, MMLV-gag-pol, and pVSV-G). GFP-positive cell lines were prepared by infecting with the retrovirus encoding GFP. A lentivirus encoding Luciferase (Luc) was prepared by transiently transfecting 293T cells with Lenti-Luciferase, pspAX2, and pVSV-Q and a retrovirus encoding GFP-firefly luciferase (GFP-FFLuc) was prepared by transiently transfecting 293T cells with GFS-GFP-FFLuc, MMLV-gag-pol, and pVSV-G. Tumor cell lines for mice were prepared by infecting cells with the lentivirus encoding Luc or the retrovirus encoding GFP-FFLuc.

### Example 3: Construction of Chimeric Antigen Receptor

A chimeric antigen receptor was composed of a single-chain antibody domain, a CD8a hinge region, a CD8a transmembrane domain, a CD28 or 4-1BB costimulatory factor domain, and a CD3 signal transduction domain (as shown in FIG. 3). The single-chain antibody (scFv) domain of the chimeric antigen receptors were derived from antibodies against B7-H3 that were produced by different monoclonal hybridoma cell clones, with the amino acid sequences as shown in SEQ ID NO: 2, 14, 26, 38, 50, 62, 74, 86, 98 or 110. The chimeric antigen receptors were generated by gene synthesis and then cloned into a retroviral vector. The correctness of the sequences of the chimeric antigen receptors were confirmed by sequencing.

TABLE 1
Sequence listing

SEQ ID NO. 1: a nucleic acid sequence of a 15C11 single-chain antibody region
ATGGAGTTTGGGCTGAGCTGGCTTTTTCTTGTGGCTATTTTAAAAGGTGTCCAGTGCTCTAGAGACATC
CAGATGACACAGAGCCCTAGCAGCCTGTCTGCCAGCGTGGGAGACAGAGTGACCATCACCTGTCAGG
CCTCTCAGAACGTGCGGACAGCCGTGGCTTGGTATCAGCAGAAGCCTGGCAAGGCCCCTAAGGCTCTG
ATCTACCTGCCTAGCAACAGACTGACCGGCGTGCCCTCTAGATTCAGCGGCTCTGGCTCTGGCACCGA
CTTCACCTTCACAATCAGCAGCCTGCAGCCTGAGGATATCGCTACCTACTTCTGCCTGCAACACTGGAA
CTACCCTTTCACCTTCGGCCAGGGCACCAAGCTGGAAATCAAGGGTGGTGGTGGTTCTGGTGGTGGTG
GTTCTGGCGGCGGCGGCTCCGGTGGTGGTGGATCCGAGGTTCAGCTGCAGCAGTCTGGACCTGAGCTG
GTTAAGCCTGGCGCCTCCGTGAAGATCCCTTGCAAGACAAGCGGCTACATCTTCACCAGCTACTGGATC
CACTGGGTCAAGCAGAGCCACGGCAAGAGCCTGGAATGGATCGGCAGAATCTACCCCGGCGTGGACA
GCATCTACTACAACGAGAAGTTCAAGGGCAAAGCCACACTGACCGCCGACAAGAGCAGCAGCACAGC
CTACATGGAACTGAGAAGCCTGACCAGCGAGGATACCGCCGTGTACTTCTGTGCCACCATCAGCTACG
ACTACGATTACAGCATGGACTACTGGGGCCAGGGCACCACCGTGACAGTTTCTTCT
SEQ ID NO. 2: a protein sequence of the 15C11 single-chain antibody region
MEFGLSWLFLVAILKGVQCSRDIQMTQSPSSLSASVGDRVTITCQASQNVRTAVAWYQQKPGKAPKALIYL
PSNRLTGVPSRFSGSGSGTDFTFTISSLQPEDIATYFCLQHWNYPFTFGQGTKLEIKGGGGSGGGGSGGGGS
GGGGSEVQLQQSGPELVKPGASVKIPCKTSGYIFTSYWIHWVKQSHGKSLEWIGRIYPGVDSIYYNEKFKG
KATLTADKSSSTAYMELRSLTSEDTAVYFCATISYDYDYSMDYWGQGTTVTVSS
SEQ ID NO. 3: a nucleic acid sequence of a light chain of the 15C11 single-chain antibody
GACATCCAGATGACACAGAGCCCTAGCAGCCTGTCTGCCAGCGTGGGAGACAGAGTGACCATCACCT
GTCAGGCCTCTCAGAACGTGCGGACAGCCGTGGCTTGGTATCAGCAGAAGCCTGGCAAGGCCCCTAA
GGCTCTGATCTACCTGCCTAGCAACAGACTGACCGGCGTGCCCTCTAGATTCAGCGGCTCTGGCTCTGG
CACCGACTTCACCTTCACAATCAGCAGCCTGCAGCCTGAGGATATCGCTACCTACTTCTGCCTGCAACA
CTGGAACTACCCTTTCACCTTCGGCCAGGGCACCAAGCTGGAAATCAAG
SEQ ID NO. 4: a protein sequence of the light chain of the 15C11 single-chain antibody
DIQMTQSPSSLSASVGDRVTITCQASQNVRTAVAWYQQKPGKAPKALIYLPSNRLTGVPSRFSGSGSGTDFT
FTISSLQPEDIATYFCLQHWNYPFTFGQGTKLEIK
SEQ ID NO. 5: a protein sequence of CDR1 of the light chain of the 15C11 single-chain antibody
QASQNVRTAVA
SEQ ID NO. 6: a protein sequence of CDR2 of the light chain of the 15C11 single-chain antibody
LPSNRLT
SEQ ID NO. 7: a protein sequence of CDR3 of the light chain of the 15C11 single-chain antibody
LQHWNYPFT
SEQ ID NO. 8: a nucleic acid sequence of a heavy chain of the 15C11 single-chain antibody
GAGGTTCAGCTGCAGCAGTCTGGACCTGAGCTGGTTAAGCCTGGCGCCTCCGTGAAGATCCCTTGCAA
GACAAGCGGCTACATCTTCACCAGCTACTGGATCCACTGGGTCAAGCAGAGCCACGGCAAGAGCCTG
GAATGGATCGGCAGAATCTACCCCGGCGTGGACAGCATCTACTACAACGAGAAGTTCAAGGGCAAAGC
CACACTGACCGCCGACAAGAGCAGCAGCACAGCCTACATGGAACTGAGAAGCCTGACCAGCGAGGAT
ACCGCCGTGTACTTCTGTGCCACCATCAGCTACGACTACGATTACAGCATGGACTACTGGGGCCAGGGC
ACCACCGTGACAGTTTCTTCT
SEQ ID NO. 9: a protein sequence of a heavy chain of the 15C11 single-chain antibody
QLQQSGPELVKPGASVKIPCKTSGYIFTSYWIHWVKQSHGKSLEWIGRIYPGVDSIYYNEKFKGKATLTAD
KSSSTAYMELRSLTSEDTAVYFCATISYDYDYSMDYWGQGTTVTVSS
SEQ ID NO. 10: a protein sequence of CDR1 of the heavy chain of the 15C11 single-chain antibody
GYIFTSYWIH
SEQ ID NO. 11: a protein sequence of CDR2 of the heavy chain of the 15C11 single-chain antibody
RIYPGVDSIYYNEKFKG
SEQ ID NO. 12: a protein sequence of CDR3 of the heavy chain of the 15C11 single-chain antibody
ATISYDYDYSMDY
SEQ ID NO. 13: a nucleic acid sequence of a 53E9D7 single-chain antibody
ATGGAGTTTGGGCTGAGCTGGCTTTTTCTTGTGGCTATTTTAAAAGGTGTCCAGTGCTCTAGAGATGTT
GTGCTAACTCAGTCTCCAGCCACCCTGTCTGTGACTCCAGGAGATAGCGTCAGTCTTTCCTGCAGGGCC
AGCCAAAGTATTAGCATCAACCTACACTGGTATCAACAAAAATCACATGAGTCTCCAAGGCTTCTCATT
AAGTATGCTTCCCAGTCCATCTCTGGGATCCCCTCCAGGTTCAGTGGCAGTGGATCAGGGACAGATTTC
ACTCTCAGTATCCACAGTGTGGAGACTGAAGATTTTGGAATGTTTTTCTGTCAACACAGTAACAGCTGG
CCTCTCACGTTCGGTGCTGGGACCAAGCTGGAAATCAAAGGTGGTGGTGGTTCTGGTGGTGGTGGTTC
TGGCGGCGGCGGCTCCGGTGGTGGTGGATCCCAGGTTACTCTGAAAGAGTCTGGCCCTGGGATATTGC
AGCCCTCCCAGACCCTCAGTCTGACTTGTTCTTTCTCTGGGTTTTCACTGAGCACTTCTGGTATGGGTG
TTGGCTGGATTCGTCAGCCATCACGGAAGGGTCTGGAGTGGCTGGCACACATTTGGTGGGATGATGTC
AAGCGCTATAACCCAGCCCTGAAGAGCCGACTGACTATCTCCAAGGATACCTCCAGCAGCCAGGTGTT
CCTCAAGATCGCCAGTGTGGACACTGCGGATACTGCCACATACTACTGTGCTCGAATACCCCACTGGTA
CTTCGATGTCTGGGGCGCAGGGACCACGGTCACCGTCTCCTCA
SEQ ID NO. 14: a protein sequence of the 53E9D7 single-chain antibody
MEFGLSWLFLVAILKGVQCSRDVVLTQSPATLSVTPGDSVSLSCRASQSISINLHWYQQKSHESPRLLIKYAS
QSISGIPSRFSGSGSGTDFTLSIHSVETEDFGMFFCQHSNSWPLTFGAGTKLEIKGGGGSGGGGSGGGGSGG
GGSQVTLKESGPGILQPSQTLSLTCSFSGFSLSTSGMGVGWIRQPSRKGLEWLAHIWWDDVKRYNPALKSR
LTISKDTSSSQVFLKIASVDTADTATYYCARIPHWYFDVWGAGTTVTVSS
SEQ ID NO. 15: a nucleic acid sequence of a light chain of the 53E9D7 single-chain antibody
GATGTTGTGCTAACTCAGTCTCCAGCCACCCTGTCTGTGACTCCAGGAGATAGCGTCAGTCTTTCCTGC
AGGGCCAGCCAAAGTATTAGCATCAACCTACACTGGTATCAACAAAAATCACATGAGTCTCCAAGGCTT
CTCATTAAGTATGCTTCCCAGTCCATCTCTGGGATCCCCTCCAGGTTCAGTGGCAGTGGATCAGGGACA
GATTTCACTCTCAGTATCCACAGTGTGGAGACTGAAGATTTTGGAATGTTTTTCTGTCAACACAGTAAC
AGCTGGCCTCTCACGTTCGGTGCTGGGACCAAGCTGGAAATCAAA
SEQ ID NO. 16: a protein sequence of the light chain of the 53E9D7 single-chain antibody
DVVLTQSPATLSVTPGDSVSLSCRASQSISINLHWYQQKSHESPRLLIKYASQSISGIPSRFSGSGSGTDFTLSI
HSVETEDFGMFFCQHSNSWPLTFGAGTKLEIK
SEQ ID NO. 17: a protein sequence of CDR1 of the light chain of the 53E9D7 single-chain antibody
RASQSISINLH
SEQ ID NO. 18: a protein sequence of CDR2 of the light chain of the 53E9D7 single-chain antibody
YASQSIS
SEQ ID NO. 19: a protein sequence of CDR3 of the light chain of the 53E9D7 single-chain antibody
QHSNSWPLT
SEQ ID NO. 20: a nucleic acid sequence of a heavy chain of the 53E9D7 single-chain antibody
CAGGTTACTCTGAAAGAGTCTGGCCCTGGGATATTGCAGCCCTCCCAGACCCTCAGTCTGACTTGTTCT
TTCTCTGGGTTTTCACTGAGCACTTCTGGTATGGGTGTTGGCTGGATTCGTCAGCCATCACGGAAGGGT
CTGGAGTGGCTGGCACACATTTGGTGGGATGATGTCAAGCGCTATAACCCAGCCCTGAAGAGCCGACT
GACTATCTCCAAGGATACCTCCAGCAGCCAGGTGTTCCTCAAGATCGCCAGTGTGGACACTGCGGATA
CTGCCACATACTACTGTGCTCGAATACCCCACTGGTACTTCGATGTCTGGGGCGCAGGGACCACGGTCA
CCGTCTCCTCA
SEQ ID NO. 21: a protein sequence of the heavy chain of the 53E9D7 single-chain antibody
QVTLKESGPGILQPSQTLSLTCSFSGFSLSTSGMGVGWIRQPSRKGLEWLAHIWWDDVKRYNPALKSRLTIS
KDTSSSQVFLKIASVDTADTATYYCARIPHWYFDVWGAGTTVTVSS
SEQ ID NO. 22: a protein sequence of CDR1 of the heavy chain of the 53E9D7 single-chain antibody
GFSLSTSGMGVG
SEQ ID NO. 23: a protein sequence of CDR2 of the heavy chain of the 53E9D7 single-chain antibody
HIWWDDVKRYNPALKS
SEQ ID NO. 24: a protein sequence of CDR3 of the heavy chain of the 53E9D7 single-chain antibody
ARIPHWYFDV
SEQ ID NO. 25: a nucleic acid sequence of a 15C11C3 single-chain antibody
ATGGAGTTTGGGCTGAGCTGGCTTTTTCTTGTGGCTATTTTAAAAGGTGTCCAGTGCTCTAGAGACATT
GTGATGACCCAGTCTCAAAAATTCATGTCCACATCAGTAGGAGACAGGGTCAGCATCACCTGCAAGGC
CAGTCAGAATGTTCGTACTGCTGTAGCCTGGTATCAACAGAAACCAGGCCAGTCTCCTAAAGCACTGAT
TTACTTGCCATCCAACCGGCTCACTGGAGTCCCTGATCGCTTCACAGGCAGTGGATCTGGGACAGATTT
CACTCTCACCATTAGCAATGTCCAATCTGAAGACCTGGCAGATTATTTCTGTCTGCAACATTGGAATTAT
CCATTCACGTTCGGCTCGGGGACAAAGTTGGAAATAAAAGGTGGTGGTGGTTCTGGTGGTGGTGGTTC
TGGCGGCGGCGGCTCCGGTGGTGGTGGATCCCAGGTCCAGCTGAAGCAGTCTGGAGCTGAGCTGGTG
AGGCCTGGGGCTTCAGTGAATCTGTCCTGCAAGACTTCTGGATACATCTTCACCAGCTACTGGATTCAC
TGGGTAAAACAGAGGTCTGGACAGGGCCTTGAGTGGATTGCAAGGATTTATCCTGGAGTTGATAGTATT
TATTACAATGAGAAGTTCAAGGGCAAGGCCACACTGACTGCAGACAAATCCTCCAGCACTGCCTACAT
GCAGCTCAGCAGCCTGAAATCTGAGGACTCTGCTGTCTATTTCTGTGCAACTATCTCCTATGATTACGAC
TATTCTATGGACTACTGGGGTCAAGGAACCTCAGTCACCGTCTCCTCA
SEQ ID NO. 26: a protein sequence of the 15C11C3 single-chain antibody
MEFGLSWLFLVAILKGVQCSRDIVMTQSQKFMSTSVGDRVSITCKASQNVRTAVAWYQQKPGQSPKALIYL
PSNRLTGVPDRFTGSGSGTDFTLTISNVQSEDLADYFCLQHWNYPFTFGSGTKLEIKGGGGSGGGGSGGGG
SGGGGSQVQLKQSGAELVRPGASVNLSCKTSGYIFTSYWIHWVKQRSGQGLEWIARIYPGVDSIYYNEKF
KGKATLTADKSSSTAYMQLSSLKSEDSAVYFCATISYDYDYSMDYWGQGTSVTVSS
SEQ ID NO. 27: a nucleic acid sequence of the 15C11C3 single-chain antibody
GACATTGTGATGACCCAGTCTCAAAAATTCATGTCCACATCAGTAGGAGACAGGGTCAGCATCACCTG
CAAGGCCAGTCAGAATGTTCGTACTGCTGTAGCCTGGTATCAACAGAAACCAGGCCAGTCTCCTAAAG
CACTGATTTACTTGCCATCCAACCGGCTCACTGGAGTCCCTGATCGCTTCACAGGCAGTGGATCTGGGA
CAGATTTCACTCTCACCATTAGCAATGTCCAATCTGAAGACCTGGCAGATTATTTCTGTCTGCAACATTG
GAATTATCCATTCACGTTCGGCTCGGGGACAAAGTTGGAAATAAAA
SEQ ID NO. 28: a protein sequence of a light chain of the 15C11C3 single-chain antibody
DIVMTQSQKFMSTSVGDRVSITCKASQNVRTAVAWYQQKPGQSPKALIYLPSNRLTGVPDRFTGSGSGTDF
TLTISNVQSEDLADYFCLQHWNYPFTFGSGTKLEIK
SEQ ID NO. 29: a protein sequence of CDR1 of the light chain of the 15C11C3 single-chain antibody
KASQNVRTAVA
SEQ ID NO. 30: a protein sequence of CDR2 of the light chain of the 15C11C3 single-chain antibody
LPSNRLT
SEQ ID NO. 31: a protein sequence of CDR3 of the light chain of the 15C11C3 single-chain antibody
LQHWNYPFT
SEQ ID NO. 32: a nucleic acid sequence of a heavy chain of the 15C11C3 single-chain antibody
CAGGTCCAGCTGAAGCAGTCTGGAGCTGAGCTGGTGAGGCCTGGGGCTTCAGTGAATCTGTCCTGCA
AGACTTCTGGATACATCTTCACCAGCTACTGGATTCACTGGGTAAAACAGAGGTCTGGACAGGGCCTT
GAGTGGATTGCAAGGATTTATCCTGGAGTTGATAGTATTTATTACAATGAGAAGTTCAAGGGCAAGGCC
ACACTGACTGCAGACAAATCCTCCAGCACTGCCTACATGCAGCTCAGCAGCCTGAAATCTGAGGACTC
TGCTGTCTATTTCTGTGCAACTATCTCCTATGATTACGACTATTCTATGGACTACTGGGGTCAAGGAACCT
CAGTCACCGTCTCCTCA
SEQ ID NO. 33: a protein sequence of the heavy chain of the 15C11C3 single-chain antibody
QVQLKQSGAELVRPGASVNLSCKTSGYIFTSYWIHWVKQRSGQGLEWIARIYPGVDSIYYNEKFKGKATLT
ADKSSSTAYMQLSSLKSEDSAVYFCATISYDYDYSMDYWGQGTSVTVSS
SEQ ID NO. 34: a protein sequence of CDR1 of the heavy chain of the 15C11C3 single-chain antibody
GYIFTSYWIH
SEQ ID NO. 35: a protein sequence of CDR2 of the heavy chain of the 15C11C3 single-chain antibody
RIYPGVDSIYYNEKFKG
SEQ ID NO. 36: a protein sequence of CDR3 of the heavy chain of the 15C11C3 single-chain antibody
ATISYDYDYSMDY
SEQ ID NO. 37: a nucleic acid sequence of a 6E4D9 single-chain antibody
ATGGAGTTTGGGCTGAGCTGGCTTTTTCTTGTGGCTATTTTAAAAGGTGTCCAGTGCTCTAGACAAATT
GTTCTCACCCAGTCTCCAGCAATCATGTCTGCATCTCCAGGGGAGAAGGTCACCATGACCTGCAGTGC
CAGCTCAAGTGTAAGTTACATGCACTGGTACCAGCAGAAGTCAGGCACCTCCCCCAAAAGATGGATTT
ATGACACATCCAAACTGGCTTCTGGAGTCCCTGCTCGCTTCAGTGGCAGTGGGTCTGGGACCTCTTACT
CTCTCACAATCAGCAGCATGGAGGCTGAAGATGCTGCCACTTATTACTGCCTGCAGTGGAGTAGTAACC
CGCTCACGTTCGGTGCTGGGACCAAGCTGGAAATCAAAGGTGGTGGTGGTTCTGGTGGTGGTGGTTCT
GGCGGCGGCGGCTCCGGTGGTGGTGGATCCGAGGTCCAGCTGCAGCAGTCTGGACCTGAGCTGGTGA
AGCCTGGGGCTTCAGTGAAGATATCCTGCAAGACTTCTGGATACACATTCACTGAATACACCATGCACT
GGGTGAAGCAGAGCCATGGAAAGAGCCCTGAGTGGATTGGAGGTATTAATCCTAACAATGGTGGTACT
ACCTACAACCAGAAATTCAAGGGCAAGGCCACATTGACTGTAGACAAGTCCTCCAGCACAGCCTACAT
GGAGCTCCGCAGCCTGACATCTGAGGATTCTGCAGTCTATTACTGTGCGAGACGGATTACTACGATGAT
ACCTACGGGGGGCTTTGACTACTGGGGCCAAGGCACCACTCTCACAGTCTCCTCA
SEQ ID NO. 38: a protein sequence of the 6E4D9 single-chain antibody
MEFGLSWLFLVAILKGVQCSRQIVLTQSPAIMSASPGEKVTMTCSASSSVSYMHWYQQKSGTSPKRWIYDT
SKLASGVPARFSGSGSGTSYSLTISSMEAEDAATYYCLQWSSNPLTFGAGTKLEIKGGGGSGGGGSGGGGS
GGGGSEVQLQQSGPELVKPGASVKISCKTSGYTFTEYTMHWVKQSHGKSPEWIGGINPNNGGTTYNQKFK
GKATLTVDKSSSTAYMELRSLTSEDSAVYYCARRITTMIPTGGFDYWGQGTTLTVSS
SEQ ID NO. 39: a nucleic acid sequence of the 6E4D9 single-chain antibody
CAAATTGTTCTCACCCAGTCTCCAGCAATCATGTCTGCATCTCCAGGGGAGAAGGTCACCATGACCTGC
AGTGCCAGCTCAAGTGTAAGTTACATGCACTGGTACCAGCAGAAGTCAGGCACCTCCCCCAAAAGATG
GATTTATGACACATCCAAACTGGCTTCTGGAGTCCCTGCTCGCTTCAGTGGCAGTGGGTCTGGGACCTC
TTACTCTCTCACAATCAGCAGCATGGAGGCTGAAGATGCTGCCACTTATTACTGCCTGCAGTGGAGTAG
TAACCCGCTCACGTTCGGTGCTGGGACCAAGCTGGAAATCAAA
SEQ ID NO. 40: a protein sequence of a light chain of the 6E4D9 single-chain antibody
QIVLTQSPAIMSASPGEKVTMTCSASSSVSYMHWYQQKSGTSPKRWIYDTSKLASGVPARFSGSGSGTSYS
LTISSMEAEDAATYYCLQWSSNPLTFGAGTKLEIK
SEQ ID NO. 41: a protein sequence of CDR1 of the light chain of the 6E4D9 single-chain antibody
SASSSVSYMH
SEQ ID NO. 42: a protein sequence of CDR2 of the light chain of the 6E4D9 single-chain antibody
DTSKLAS
SEQ ID NO. 43: a protein sequence of CDR3 of the light chain of the 6E4D9 single-chain antibody
LQWSSNPLT
SEQ ID NO. 44: a nucleic acid sequence of a heavy chain of the 6E4D9 single-chain antibody
GAGGTCCAGCTGCAGCAGTCTGGACCTGAGCTGGTGAAGCCTGGGGCTTCAGTGAAGATATCCTGCAA
GACTTCTGGATACACATTCACTGAATACACCATGCACTGGGTGAAGCAGAGCCATGGAAAGAGCCCTG
AGTGGATTGGAGGTATTAATCCTAACAATGGTGGTACTACCTACAACCAGAAATTCAAGGGCAAGGCCA
CATTGACTGTAGACAAGTCCTCCAGCACAGCCTACATGGAGCTCCGCAGCCTGACATCTGAGGATTCTG
CAGTCTATTACTGTGCGAGACGGATTACTACGATGATACCTACGGGGGGCTTTGACTACTGGGGCCAAG
GCACCACTCTCACAGTCTCCTCA
SEQ ID NO. 45: a protein sequence of the heavy chain of the 6E4D9 single-chain antibody
EVQLQQSGPELVKPGASVKISCKTSGYTFTEYTMHWVKQSHGKSPEWIGGINPNNGGTTYNQKFKGKATL
TVDKSSSTAYMELRSLTSEDSAVYYCARRITTMIPTGGFDYWGQGTTLTVSS
SEQ ID NO. 46: a protein sequence of CDR1 of the heavy chain of the 6E4D9 single-chain antibody
GYTFTEYTMH
SEQ ID NO. 47: a protein sequence of CDR2 of the heavy chain of the 6E4D9 single-chain antibody
GINPNNGGTTYNQKFKG
SEQ ID NO. 48: a protein sequence of CDR3 of the heavy chain of the 6E4D9 single-chain antibody
ARRITTMIPTGGFDY
SEQ ID NO. 49: a nucleic acid sequence of a 31F3E5 single-chain antibody
ATGGAGTTTGGGCTGAGCTGGCTTTTTCTTGTGGCTATTTTAAAAGGTGTCCAGTGCTCTAGAGAAATT
TTGCTCACCCAGTCTCCAGCAATCATAGCTGTATCTCCTGGGGAGAAGGTCACCATAACCTGCAGTGCC
AGCTCAAGTGTAAATTACATGAACTGGTACCAACAGAAACCAGGATCCTCCCCCAAAATATGGATTTTT
GGTATGTCCAACCTGGCTTCTGGAGTTCCTGCTCGCTTCAGTGGCAGTGGGTCTGGGACATCTTTCTCT
TTCACAATCAACAGCATGGAGGCTGAAGATGTTGCCACTTATTACTGTCACCAAAGGAGTAGTTACCCA
TACACGTTCGGAGGGGGGACCAAGCTGGAAATCAAAGGTGGTGGTGGTTCTGGTGGTGGTGGTTCTG
GCGGCGGCGGCTCCGGTGGTGGTGGATCCCAGATCCAGTTGGTGCAGTCTGGACCTGAATTGAAGAAG
CCTGGAGAGACAGTCAGGATCTCCTGCAAGGCTTCTGGATATACCTTCACAAACTATGGAATGAACTGG
GTGAAGCAGGCTCCAGGAAAGGGTTTGAAGTGGATGGGCTGGATTAATACCTACACTGGAGAGCCGA
CATATTCTGATGTCTTCAGGGGACGGTTTGCCTTCTCTTTGGAAACCTCTGCCAACACTGCCTATTTAGA
GATCAGAAAACTCAAAAATGAGGACATGGCTACGTATTTCTGTGTGAGAAATGAATACGACGCACCTTA
CTGGGGCCAAGGGACTCTGGTCACTGTCTCTGCA
SEQ ID NO. 50: a protein sequence of the 31F3E5 single-chain antibody
MEFGLSWLFLVAILKGVQCSREILLTQSPAIIAVSPGEKVTITCSASSSVNYMNWYQQKPGSSPKIWIFGMSN
LASGVPARFSGSGSGTSFSFTINSMEAEDVATYYCHQRSSYPYTFGGGTKLEIKGGGGSGGGGSGGGGSGG
GGSQIQLVQSGPELKKPGETVRISCKASGYTFTNYGMNWVKQAPGKGLKWMGWINTYTGEPTYSDVFRG
RFAFSLETSANTAYLEIRKLKNEDMATYFCVRNEYDAPYWGQGTLVTVSA
SEQ ID NO. 51: a nucleic acid sequence of a light chain of the 31F3E5 single-chain antibody
GAAATTTTGCTCACCCAGTCTCCAGCAATCATAGCTGTATCTCCTGGGGAGAAGGTCACCATAACCTGC
AGTGCCAGCTCAAGTGTAAATTACATGAACTGGTACCAACAGAAACCAGGATCCTCCCCCAAAATATG
GATTTTTGGTATGTCCAACCTGGCTTCTGGAGTTCCTGCTCGCTTCAGTGGCAGTGGGTCTGGGACATC
TTTCTCTTTCACAATCAACAGCATGGAGGCTGAAGATGTTGCCACTTATTACTGTCACCAAAGGAGTAG
TTACCCATACACGTTCGGAGGGGGGACCAAGCTGGAAATCAAA
SEQ ID NO. 52: a protein sequence of a light chain of the 31F3E5 single-chain antibody
EILLTQSPAIIAVSPGEKVTITCSASSSVNYMNWYQQKPGSSPKIWIFGMSNLASGVPARFSGSGSGTSFSFTI
NSMEAEDVATYYCHQRSSYPYTFGGGTKLEIK
SEQ ID NO. 53: a protein sequence of CDR1 of the light chain of the 31F3E5 single-chain antibody
SASSSVNYMN
SEQ ID NO. 54: a protein sequence of CDR2 of the light chain of the 31F3E5 single-chain antibody
GMSNLAS
SEQ ID NO. 55: a protein sequence of CDR3 of the light chain of the 31F3E5 single-chain antibody
HQRSSYPYT
SEQ ID NO. 56: a nucleic acid sequence of a heavy chain of the 31F3E5 single-chain antibody
CAGATCCAGTTGGTGCAGTCTGGACCTGAATTGAAGAAGCCTGGAGAGACAGTCAGGATCTCCTGCAA
GGCTTCTGGATATACCTTCACAAACTATGGAATGAACTGGGTGAAGCAGGCTCCAGGAAAGGGTTTGA
AGTGGATGGGCTGGATTAATACCTACACTGGAGAGCCGACATATTCTGATGTCTTCAGGGGACGGTTTG
CCTTCTCTTTGGAAACCTCTGCCAACACTGCCTATTTAGAGATCAGAAAACTCAAAAATGAGGACATGG
CTACGTATTTCTGTGTGAGAAATGAATACGACGCACCTTACTGGGGCCAAGGGACTCTGGTCACTGTCT
CTGCA
SEQ ID NO. 57: a protein sequence of the heavy chain of the 31F3E5 single-chain antibody
QIQLVQSGPELKKPGETVRISCKASGYTFTNYGMNWVKQAPGKGLKWMGWINTYTGEPTYSDVFRGRFA
FSLETSANTAYLEIRKLKNEDMATYFCVRNEYDAPYWGQGTLVTVSA
SEQ ID NO. 58: a protein sequence of CDR1 of the heavy chain of the 31F3E5 single-chain antibody
GYTFTNYGMN
SEQ ID NO. 59: a protein sequence of CDR2 of the heavy chain of the 31F3E5 single-chain antibody
WINTYTGEPTYSDVFRG
SEQ ID NO. 60: a protein sequence of CDR3 of the heavy chain of the 31F3E5 single-chain antibody
VRNEYDAPY
SEQ ID NO. 61: a nucleic acid sequence of a 21H2E11 single-chain antibody
ATGGAGTTTGGGCTGAGCTGGCTTTTTCTTGTGGCTATTTTAAAAGGTGTCCAGTGCTCTAGAAACATT
GTGTTGGCCCAATCTCCAGCTTCTTTGGCTGTGTCTCTAGGGCAGAGGGCCACCATATCCTGCAGAGCC
AGCGAAAGTGTTGATAATTATGGCTATAGTTTTATGCACTGGTACCAGCAGAAACCAGGACAGTCACCC
AAACTCCTCATCTATCTTGCATCCAACCTAGAATCTGGGGTCCCTGCCAGGTTCAGTGGCAGTGGGTCT
AGGTCAGACTTCACCCTCACCATTGATCCTGTGGAGGCTGATGATGCTGCAACCTATTACTGTCAGCAA
AATAGTGAAGATCCGTGGACGTTCGGTGGAGGCACCAGGCTGGAAATCAAAGGTGGTGGTGGTTCTG
GTGGTGGTGGTTCTGGCGGCGGCGGCTCCGGTGGTGGTGGATCCCAGGTTCAACTCCAGCAGTCTGGG
GCTGTGCTGGTGAAGCCTGGGGCCTCAGTGAAGATGTCCTGCAAGGCTTCTGGCTACACATTTACCAG
TTACAATTTGCACTGGATAAAGCAGACACCTGGACAGGGCCTGGAATGGATTGGAGTTATTTATTCAGG
AAATAGTGATACTTCAACCAATCAGAAGTTCAAAGGCAAGGCCACATTGACTGAAGACAAATCCGCCA
GTACAGTCTACATATATCTCAGCAGCCTGACATCTGAAGACTCTGCGGTCTATTACTGTGCAAGGGGGG
AATATGGTAACCACGGGGGATTTGCTTACTGGGGCCAAGGGACTCTGGTCACTGTCTCTGCA
SEQ ID NO. 62: a protein sequence of the 21H2E11 single-chain antibody
MEFGLSWLFLVAILKGVQCSRNIVLAQSPASLAVSLGQRATISCRASESVDNYGYSFMHWYQQKPGQSPKL
LIYLASNLESGVPARFSGSGSRSDFTLTIDPVEADDAATYYCQQNSEDPWTFGGGTRLEIKGGGGSGGGGS
GGGGSGGGGSQVQLQQSGAVLVKPGASVKMSCKASGYTFTSYNLHWIKQTPGQGLEWIGVIYSGNSDTS
TNQKFKGKATLTEDKSASTVYIYLSSLTSEDSAVYYCARGEYGNHGGFAYWGQGTLVTVSA
SEQ ID NO. 63: a nucleic acid sequence of the 21H2E11 single-chain antibody
AACATTGTGTTGGCCCAATCTCCAGCTTCTTTGGCTGTGTCTCTAGGGCAGAGGGCCACCATATCCTGC
AGAGCCAGCGAAAGTGTTGATAATTATGGCTATAGTTTTATGCACTGGTACCAGCAGAAACCAGGACAG
TCACCCAAACTCCTCATCTATCTTGCATCCAACCTAGAATCTGGGGTCCCTGCCAGGTTCAGTGGCAGT
GGGTCTAGGTCAGACTTCACCCTCACCATTGATCCTGTGGAGGCTGATGATGCTGCAACCTATTACTGT
CAGCAAAATAGTGAAGATCCGTGGACGTTCGGTGGAGGCACCAGGCTGGAAATCAAA
SEQ ID NO. 64: a protein sequence of a light chain of the 21H2E11 single-chain antibody
NIVLAQSPASLAVSLGQRATISCRASESVDNYGYSFMHWYQQKPGQSPKLLIYLASNLESGVPARFSGSGSR
SDFTLTIDPVEADDAATYYCQQNSEDPWTFGGGTRLEIK
SEQ ID NO. 65: a protein sequence of CDR1 of the light chain of the 21H2E11 single-chain antibody
RASESVDNYGYSFMH
SEQ ID NO. 66: a protein sequence of CDR2 of the light chain of the 21H2E11 single-chain antibody
LASNLES
SEQ ID NO. 67: a protein sequence of CDR3 of the light chain of the 21H2E11 single-chain antibody
QQNSEDPWT
SEQ ID NO. 68: a nucleic acid sequence of a heavy chain of the 21H2E11 single-chain antibody
CAGGTTCAACTCCAGCAGTCTGGGGCTGTGCTGGTGAAGCCTGGGGCCTCAGTGAAGATGTCCTGCAA
GGCTTCTGGCTACACATTTACCAGTTACAATTTGCACTGGATAAAGCAGACACCTGGACAGGGCCTGG
AATGGATTGGAGTTATTTATTCAGGAAATAGTGATACTTCAACCAATCAGAAGTTCAAAGGCAAGGCCA
CATTGACTGAAGACAAATCCGCCAGTACAGTCTACATATATCTCAGCAGCCTGACATCTGAAGACTCTG
CGGTCTATTACTGTGCAAGGGGGGAATATGGTAACCACGGGGGATTTGCTTACTGGGGCCAAGGGACT
CTGGTCACTGTCTCTGCA
SEQ ID NO. 69: a protein sequence of a heavy chain of the 21H2E11 single-chain antibody
QVQLQQSGAVLVKPGASVKMSCKASGYTFTSYNLHWIKQTPGQGLEWIGVIYSGNSDTSTNQKFKGKATL
TEDKSASTVYIYLSSLTSEDSAVYYCARGEYGNHGGFAYWGQGTLVTVSA
SEQ ID NO. 70: a protein sequence of CDR1 of the heavy chain of the 21H2E11 single-chain antibody
GYTFTSYNLH
SEQ ID NO. 71: a protein sequence of CDR2 of the heavy chain of the 21H2E11 single-chain antibody
VIYSGNSDTSTNQKFKG
SEQ ID NO. 72: a protein sequence of CDR3 of the heavy chain of the 21H2E11 single-chain antibody
ARGEYGNHGGFAY
SEQ ID NO. 73: a nucleic acid sequence of a 24A11A11 single-chain antibody
ATGGAGTTTGGGCTGAGCTGGCTTTTTCTTGTGGCTATTTTAAAAGGTGTCCAGTGCTCTAGAGACATT
GTGATGACCCAGTCTCAAAAATTCATGTCCACATCAGTAGGAGACAGGGTCAGCATCACCTGCAAGGC
CAGTCAGAATGTTCGTACTGCTGTAGCCTGGTATCAACAGAAACCAGGCCAGTCTCCTAAAGCACTGAT
TTACTTGCCATCCAACCGGCACACTGGAGTCCCTGATCGCTTCACAGGCAGTGGATCTGGGACAGATTT
CACTCTCACCATTAGCAATGTGCAATCTGAAGACCTGGCAGATTATTTCTGTCTGCAACATTGGAATTAT
CCATTCACGTTCGGCTCGGGGACAAAGTTGGAAATAAAAGGTGGTGGTGGTTCTGGTGGTGGTGGTTC
TGGCGGCGGCGGCTCCGGTGGTGGTGGATCCCAGGTCCAGCTGAAGCAGTCTGGAGCTGAGCTGGTG
AGGCCTGGGGCTTCAGTGAATCTGTCCTGCAAGACTTCTGGATACATCTTCACCAGCTACTGGATTCAC
TGGGTAAAACAGAGGTCTGGACAGGGCCTTGAGTGGATTGCAAGGATTTATCCTGGAGTTGATAGTATT
TATTACAATGAGAAGTTCAAGGGCAAGGCCACACTGACTGCAGACAAATCCTCCAGCACTGCCTACAT
GCAGCTCAGCAGCCTGAAATCTGAGGACTCTGCTGTCTATTTCTGTGCAACTATCTCCTATGATTACGAC
TATTCTATGGACTACTGGGGTCAAGGAACCTCAGTCACCGTCTCCTCA
SEQ ID NO. 74: a protein sequence of the 24A11A11 single-chain antibody
MEFGLSWLFLVAILKGVQCSRDIVMTQSQKFMSTSVGDRVSITCKASQNVRTAVAWYQQKPGQSPKALIYL
PSNRHTGVPDRFTGSGSGTDFTLTISNVQSEDLADYFCLQHWNYPFTFGSGTKLEIKGGGGSGGGGSGGGG
SGGGGSQVQLKQSGAELVRPGASVNLSCKTSGYIFTSYWIHWVKQRSGQGLEWIARIYPGVDSIYYNEKF
KGKATLTADKSSSTAYMQLSSLKSEDSAVYFCATISYDYDYSMDYWGQGTSVTVSS
SEQ ID NO. 75: a nucleic acid sequence of a light chain of the 24A11A11 single-chain antibody
GACATTGTGATGACCCAGTCTCAAAAATTCATGTCCACATCAGTAGGAGACAGGGTCAGCATCACCTG
CAAGGCCAGTCAGAATGTTCGTACTGCTGTAGCCTGGTATCAACAGAAACCAGGCCAGTCTCCTAAAG
CACTGATTTACTTGCCATCCAACCGGCACACTGGAGTCCCTGATCGCTTCACAGGCAGTGGATCTGGGA
CAGATTTCACTCTCACCATTAGCAATGTGCAATCTGAAGACCTGGCAGATTATTTCTGTCTGCAACATTG
GAATTATCCATTCACGTTCGGCTCGGGGACAAAGTTGGAAATAAAA
SEQ ID NO. 76: a protein sequence of the light chain of the 24A11A11 single-chain antibody
DIVMTQSQKFMSTSVGDRVSITCKASQNVRTAVAWYQQKPGQSPKALIYLPSNRHTGVPDRFTGSGSGTDF
TLTISNVQSEDLADYFCLQHWNYPFTFGSGTKLEIK
SEQ ID NO. 77: a protein sequence of CDR1 of the light chain of the 24A11A11 single-chain antibody
KASQNVRTAVA
SEQ ID NO. 78: a protein sequence of CDR2 of the light chain of the 24A11A11 single-chain antibody
LPSNRHT
SEQ ID NO. 79: a protein sequence of CDR3 of the light chain of the 24A11A11 single-chain antibody
LQHWNYPFT
SEQ ID NO. 80: a nucleic acid sequence of a heavy chain of the 24A11A11 single-chain antibody
AGGTCCAGCTGAAGCAGTCTGGAGCTGAGCTGGTGAGGCCTGGGGCTTCAGTGAATCTGTCCTGCAA
GACTTCTGGATACATCTTCACCAGCTACTGGATTCACTGGGTAAAACAGAGGTCTGGACAGGGCCTTG
AGTGGATTGCAAGGATTTATCCTGGAGTTGATAGTATTTATTACAATGAGAAGTTCAAGGGCAAGGCCA
CACTGACTGCAGACAAATCCTCCAGCACTGCCTACATGCAGCTCAGCAGCCTGAAATCTGAGGACTCT
GCTGTCTATTTCTGTGCAACTATCTCCTATGATTACGACTATTCTATGGACTACTGGGGTCAAGGAACCT
CAGTCACCGTCTCCTCA
SEQ ID NO. 81: a protein sequence of the heavy chain of the 24A11A11 single-chain antibody
QVQLKQSGAELVRPGASVNLSCKTSGYIFTSYWIHWVKQRSGQGLEWIARIYPGVDSIYYNEKFKGKATLT
ADKSSSTAYMQLSSLKSEDSAVYFCATISYDYDYSMDYWGQGTSVTVSS
SEQ ID NO. 82: a protein sequence of CDR1 of the heavy chain of the 24A11A11 single-chain antibody
GYIFTSYWIH
SEQ ID NO. 83: a protein sequence of CDR2 of the heavy chain of the 24A11A11 single-chain antibody
RIYPGVDSIYYNEKFKG
SEQ ID NO. 84: a protein sequence of CDR3 of the heavy chain of the 24A11A11 single-chain sequence
ATISYDYDYSMDY
SEQ ID NO. 85: a nucleic acid sequence of a 42B5C12 single-chain antibody
ATGGAGTTTGGGCTGAGCTGGCTTTTTCTTGTGGCTATTTTAAAAGGTGTCCAGTGCTCTAGAGATATTG
TGATGACGCAGGCTGCATTCTCCAATCCAGTCACTCTTGGAACATCAGCTTCCATCTCCTGCAGGTCTA
GTAAGAGTCTCCTCCATAGTAATGGCATCACTTATTTCTATTGGTATCTGCAGAAGCCAGGCCAGTCTCC
TCAGCTCCTGATTTATCAGATGTCCAGCCTTGCCTCAGGAGTCCCAGACAGGTTCAGTAGCAGTGGGTC
AGGAACTGATTTCACACTGAGAATCAGCAGAGTGGAGGCTGAGGATGTGGGTGTTTATTACTGTGCTC
AAAATCTAGAACTTCCGTACACGTTCGGAGGGGGGACCAAGCTGGAAATCAAAGGTGGTGGTGGTTCT
GGTGGTGGTGGTTCTGGCGGCGGCGGCTCCGGTGGTGGTGGATCCCAGATCCAGTTGGTGCAGTCTGG
ACCTGAGGTGAGGAGGCCTGGGGAGACAGTCAAGATCTCCTGCAAGGCTTCTGGATATACTTTCACAA
ACTTTGGAATGAACTGGGTGAAACAGGCTCCAGGAAAGGCTTTACAGTGGATGGGCTGGATAAACACC
TACACTGGTGAGCCAACATATGCTGATGACTTCAAGGGCCGGTTTGCCTTCTCTTTGGAATCCTCTGCC
AGCACTGCCCATTTGCAGATCAGCTACCTCAAAGATGAGGACACGGCTACATATTTCTGTGCGAGATGG
TGGCTTCAATATGCTTTGGACAACTGGGGTCAAGGAACCTCAGTCACCGTCTCCTCA
SEQ ID NO. 86: a protein sequence of the 42B5C12 single-chain antibody
MEFGLSWLFLVAILKGVQCSRDIVMTQAAFSNPVTLGTSASISCRSSKSLLHSNGITYFYWYLQKPGQSPQL
LIYQMSSLASGVPDRFSSSGSGTDFTLRISRVEAEDVGVYYCAQNLELPYTFGGGTKLEIKGGGGSGGGGS
GGGGSGGGGSQIQLVQSGPEVRRPGETVKISCKASGYTFTNFGMNWVKQAPGKALQWMGWINTYTGEPT
YADDFKGRFAFSLESSASTAHLQISYLKDEDTATYFCARWWLQYALDNWGQGTSVTVSS
SEQ ID NO. 87: a nucleic acid sequence of a light chain of the 42B5C12 single-chain antibody
GATATTGTGATGACGCAGGCTGCATTCTCCAATCCAGTCACTCTTGGAACATCAGCTTCCATCTCCTGCA
GGTCTAGTAAGAGTCTCCTCCATAGTAATGGCATCACTTATTTCTATTGGTATCTGCAGAAGCCAGGCCA
GTCTCCTCAGCTCCTGATTTATCAGATGTCCAGCCTTGCCTCAGGAGTCCCAGACAGGTTCAGTAGCAG
TGGGTCAGGAACTGATTTCACACTGAGAATCAGCAGAGTGGAGGCTGAGGATGTGGGTGTTTATTACT
GTGCTCAAAATCTAGAACTTCCGTACACGTTCGGAGGGGGGACCAAGCTGGAAATCAAA
SEQ ID NO. 88: a protein sequence of the light chain of the 42B5C12 single-chain antibody
DIVMTQAAFSNPVTLGTSASISCRSSKSLLHSNGITYFYWYLQKPGQSPQLLIYQMSSLASGVPDRFSSSGS
GTDFTLRISRVEAEDVGVYYCAQNLELPYTFGGGTKLEIK
SEQ ID NO. 89: a protein sequence of CDR1 of the light chain of the 42B5C12 single-chain antibody
RSSKSLLHSNGITYFY
SEQ ID NO. 90: a protein sequence of CDR2 of the light chain of the 42B5C12 single-chain antibody
QMSSLAS
SEQ ID NO. 91: a protein sequence of CDR3 of the light chain of the 42B5C12 single-chain antibody
AQNLELPYT
SEQ ID NO. 92: a nucleic acid sequence of a heavy chain of the 42B5C12 single-chain antibody
CAGATCCAGTTGGTGCAGTCTGGACCTGAGGTGAGGAGGCCTGGGGAGACAGTCAAGATCTCCTGCA
AGGCTTCTGGATATACTTTCACAAACTTTGGAATGAACTGGGTGAAACAGGCTCCAGGAAAGGCTTTA
CAGTGGATGGGCTGGATAAACACCTACACTGGTGAGCCAACATATGCTGATGACTTCAAGGGCCGGTT
TGCCTTCTCTTTGGAATCCTCTGCCAGCACTGCCCATTTGCAGATCAGCTACCTCAAAGATGAGGACAC
GGCTACATATTTCTGTGCGAGATGGTGGCTTCAATATGCTTTGGACAACTGGGGTCAAGGAACCTCAGT
CACCGTCTCCTCA
SEQ ID NO. 93: a protein sequence of the heavy chain of the 42B5C12 single-chain antibody
QIQLVQSGPEVRRPGETVKISCKASGYTFTNFGMNWVKQAPGKALQWMGWINTYTGEPTYADDFKGRFA
FSLESSASTAHLQISYLKDEDTATYFCARWWLQYALDNWGQGTSVTVSS
SEQ ID NO. 94: a protein sequence of CDR1 of the heavy chain of the 42B5C12 single-chain antibody
GYTFTNFGMN
SEQ ID NO. 95: a protein sequence of CDR2 of the heavy chain of the 42B5C12 single-chain antibody
WINTYTGEPTYADDFKG
SEQ ID NO. 96: a protein sequence of CDR3 of the heavy chain of the 42B5C12 single-chain antibody
ARWWLQYALDN
SEQ ID NO. 97: a nucleic sequence of a 14A7A8 single-chain antibody
ATGGAGTTTGGGCTGAGCTGGCTTTTTCTTGTGGCTATTTTAAAAGGTGTCCAGTGCTCTAGAAACATT
GTGCTGACCCAATCTCCAGCTTCTTTGGCTGTGTCTCTAGGGCAGAGGGCCACCATATCCTGCAGAGCC
AGTGAAAATGTTGATAATTATGGCAATAGTTTTATACACTGGTACCAGCAGAAACCAGGACAGCCACCC
AAACTCCTCATCTATCTTGCATCCAACCTAGAATCTGGGGTCCCTGCCAGGTTCAGTGGCAGTGGGTCT
AGGTCAGACTTCACCCTCACCATTGATCCTGTGGAGGCTGATGATGCTGCAACCTATTACTGTCAGCAA
AATAATGAGGATCCGTGGACGTTCGGTGGAGGCACCAAGCTGGAAATCAAAGGTGGTGGTGGTTCTGG
TGGTGGTGGTTCTGGCGGCGGCGGCTCCGGTGGTGGTGGATCCCAGGTCCAACTGCAGCAGCCTGGG
GCTGAGCTGGTGAAGCCTGGGGCCTCAGTGAAGATGTCCTGCAAGGCTTCTGGCTACATATTTACCAGT
TACAATTTGCACTGGGTAAAACAGACACCTGGACAGGGCCTGGAATGGATTGGAGTTATTTATTCAGGA
AATAGTGATACTTCCAACAATCAGAAGTTCAAAGGCAAGGCCACATTGACTGAAGACAAATCCGCCAG
CACAGCCTACCTACACCTCAGCAGCCTGACATCTGAAGACTCAGCGGTCTATTTCTGTGCAAGGGGGG
AATATGGTAACCACGGGGGGTTTGCTTACTGGGGCCAAGGGACTCTGGTCACTGTCTCTGCA
SEQ ID NO. 98: a protein sequence of the 14A7A8 single-chain antibody
MEFGLSWLFLVAILKGVQCSRNIVLTQSPASLAVSLGQRATISCRASENVDNYGNSFIHWYQQKPGQPPKLL
IYLASNLESGVPARFSGSGSRSDFTLTIDPVEADDAATYYCQQNNEDPWTFGGGTKLEIKGGGGSGGGGSG
GGGSGGGGSQVQLQQPGAELVKPGASVKMSCKASGYIFTSYNLHWVKQTPGQGLEWIGVIYSGNSDTSN
NQKFKGKATLTEDKSASTAYLHLSSLTSEDSAVYFCARGEYGNHGGFAYWGQGTLVTVSA
SEQ ID NO. 99: a nucleic sequence of a light chain of the 14A7A8 single-chain antibody
AACATTGTGCTGACCCAATCTCCAGCTTCTTTGGCTGTGTCTCTAGGGCAGAGGGCCACCATATCCTGC
AGAGCCAGTGAAAATGTTGATAATTATGGCAATAGTTTTATACACTGGTACCAGCAGAAACCAGGACAG
CCACCCAAACTCCTCATCTATCTTGCATCCAACCTAGAATCTGGGGTCCCTGCCAGGTTCAGTGGCAGT
GGGTCTAGGTCAGACTTCACCCTCACCATTGATCCTGTGGAGGCTGATGATGCTGCAACCTATTACTGT
CAGCAAAATAATGAGGATCCGTGGACGTTCGGTGGAGGCACCAAGCTGGAAATCAAA
SEQ ID NO. 100: a protein sequence of the light chain of the 14A7A8 single-chain antibody
NIVLTQSPASLAVSLGQRATISCRASENVDNYGNSFIHWYQQKPGQPPKLLIYLASNLESGVPARFSGSGSRS
DFTLTIDPVEADDAATYYCQQNNEDPWTFGGGTKLEIK
SEQ ID NO. 101: a protein sequence of CDR1 of the light chain of the 14A7A8 single-chain antibody
RASENVDNYGNSFIH
SEQ ID NO. 102: a protein sequence of CDR2 of the light chain of the 14A7A8 single-chain antibody
LASNLES
SEQ ID NO. 103: a protein sequence of CDR3 of the light chain of the 14A7A8 single-chain antibody
QQNNEDPWT
SEQ ID NO. 104: a nucleic acid sequence of a heavy chain of the 14A7A8 single-chain antibody
CAGGTCCAACTGCAGCAGCCTGGGGCTGAGCTGGTGAAGCCTGGGGCCTCAGTGAAGATGTCCTGCA
AGGCTTCTGGCTACATATTTACCAGTTACAATTTGCACTGGGTAAAACAGACACCTGGACAGGGCCTGG
AATGGATTGGAGTTATTTATTCAGGAAATAGTGATACTTCCAACAATCAGAAGTTCAAAGGCAAGGCCA
CATTGACTGAAGACAAATCCGCCAGCACAGCCTACCTACACCTCAGCAGCCTGACATCTGAAGACTCA
GCGGTCTATTTCTGTGCAAGGGGGGAATATGGTAACCACGGGGGGTTTGCTTACTGGGGCCAAGGGAC
TCTGGTCACTGTCTCTGCA
SEQ ID NO. 105: a protein sequence of the heavy chain of the 14A7A8 single-chain antibody
QVQLQQPGAELVKPGASVKMSCKASGYIFTSYNLHWVKQTPGQGLEWIGVIYSGNSDTSNNQKFKGKAT
LTEDKSASTAYLHLSSLTSEDSAVYFCARGEYGNHGGFAYWGQGTLVTVSA
SEQ ID NO. 106: a protein sequence of CDR1 of the heavy chain of the 14A7A8 single-chain antibody
GYIFTSYNLH
SEQ ID NO. 107: a protein sequence of CDR2 of the heavy chain of the 14A7A8 single-chain antibody
VIYSGNSDTSNNQKFKG
SEQ ID NO. 108: a protein sequence of CDR3 of the heavy chain of the 14A7A8 single-chain antibody
ARGEYGNHGGFAY
SEQ ID NO. 109: a nucleic acid sequence of a 41C10E7 single-chain antibody
ATGGAGTTTGGGCTGAGCTGGCTTTTTCTTGTGGCTATTTTAAAAGGTGTCCAGTGCTCTAGAGACATC
CAGATGACTCAGTCTCCAGCCTCCCTAACTGTATTTGTGGGGGAAACTGTCACCATCACATGTCGAGCA
AGTGAGAATATTTACAGTAATTTAGCGTGGTATCAGCAGAAACAGGGAAAATCTCCTCAGCTCCTGGTC
TATACTGCAACAAATTTAATAGATGGTGTGCCATCAAGGTTCAGTGGCAGTGGGTCCGGCACACAGTAT
TCCCTCAAGATCAACAGCCTGCAGTCTGAAGATTTCGGGAGTTATTACTGTCAACATTTTTGGGGTACT
CCGGTCACGTTCGGTGCTGGGACCAAGCTGGAAATCAAAGGTGGTGGTGGTTCTGGTGGTGGTGGTTC
TGGCGGCGGCGGCTCCGGTGGTGGTGGATCCCAGGTCCAACTGCAGCAGCCTGGGGCAGAACTGGTG
AAGCCTGGGGCCTCAGTGAGGATGTCCTGCAGGGCTTCTGGCTACACTTTTACCAGTTACAATTTGCAC
TGGGTAAAACAGACACCTGGACAGGGCCTGGAATGGATTGGGGCTATTCATCCAGGAGATAATTATACT
TCCTACAATCAGATTTTCAACGACAAGGCCACATTGACTGCAGACAAATCCTCCAGCACAGCCTACATG
CAGCTCAGTAGCCTGACATCTGAGGACTCTGCGGTCTATTTCTGTGCAAGGAATTACTACGGTAGTCTT
GATTACTGGGGCCAAGGCACCACTCTCACAGTCTCCTCA
SEQ ID NO. 110: a protein sequence of the 41C10E7 single-chain antibody
MEFGLSWLFLVAILKGVQCSRDIQMTQSPASLTVFVGETVTITCRASENIYSNLAWYQQKQGKSPQLLVYTA
TNLIDGVPSRFSGSGSGTQYSLKINSLQSEDFGSYYCQHFWGTPVTFGAGTKLEIKGGGGSGGGGSGGGGS
GGGGSQVQLQQPGAELVKPGASVRMSCRASGYTFTSYNLHWVKQTPGQGLEWIGAIHPGDNYTSYNQIF
NDKATLTADKSSSTAYMQLSSLTSEDSAVYFCARNYYGSLDYWGQGTTLTVSS
SEQ ID NO. I11: a nucleic acid of a light chain of the 41C10E7 single-chain antibody
GACATCCAGATGACTCAGTCTCCAGCCTCCCTAACTGTATTTGTGGGGGAAACTGTCACCATCACATGT
CGAGCAAGTGAGAATATTTACAGTAATTTAGCGTGGTATCAGCAGAAACAGGGAAAATCTCCTCAGCTC
CTGGTCTATACTGCAACAAATTTAATAGATGGTGTGCCATCAAGGTTCAGTGGCAGTGGGTCCGGCACA
CAGTATTCCCTCAAGATCAACAGCCTGCAGTCTGAAGATTTCGGGAGTTATTACTGTCAACATTTTTGG
GGTACTCCGGTCACGTTCGGTGCTGGGACCAAGCTGGAAATCAAA
SEQ ID NO. 112: a protein sequence of the light chain of the 41C10E7 single-chain antibody
DIQMTQSPASLTVFVGETVTITCRASENIYSNLAWYQQKQGKSPQLLVYTATNLIDGVPSRFSGSGSGTQYS
LKINSLQSEDFGSYYCQHFWGTPVTFGAGTKLEIK
SEQ ID NO. 113: a protein sequence of CDR1 of the light chain of the 41C10E7 single-chain antibody
RASENIYSNLA
SEQ ID NO. 114: a protein sequence of CDR2 of the light chain of the 41C10E7 single-chain antibody
TATNLI
SEQ ID NO. 115: a protein sequence of CDR3 of the light chain of the 41C10E7 single-chain antibody
QHFWGTPVT
SEQ ID NO. 116: a nucleic acid sequence of a heavy chain of the 41C10E7 single-chain antibody
CAGGTCCAACTGCAGCAGCCTGGGGCAGAACTGGTGAAGCCTGGGGCCTCAGTGAGGATGTCCTGCA
GGGCTTCTGGCTACACTTTTACCAGTTACAATTTGCACTGGGTAAAACAGACACCTGGACAGGGCCTG
GAATGGATTGGGGCTATTCATCCAGGAGATAATTATACTTCCTACAATCAGATTTTCAACGACAAGGCCA
CATTGACTGCAGACAAATCCTCCAGCACAGCCTACATGCAGCTCAGTAGCCTGACATCTGAGGACTCT
GCGGTCTATTTCTGTGCAAGGAATTACTACGGTAGTCTTGATTACTGGGGCCAAGGCACCACTCTCACA
GTCTCCTCA
SEQ ID NO. 117: a protein sequence of the heavy chain of the 41C10E7 single-chain antibody
QVQLQQPGAELVKPGASVRMSCRASGYTFTSYNLHWVKQTPGQGLEWIGAIHPGDNYTSYNQIFNDKATL
TADKSSSTAYMQLSSLTSEDSAVYFCARNYYGSLDYWGQGTTLTVSS
SEQ ID NO. 118: a protein sequence of CDR1 of the heavy chain of the 41C10E7 single-chain antibody
GYTFTSYNLH
SEQ ID NO. 119: a protein sequence of CDR2 of the heavy chain of the 41C10E7 single-chain antibody
AIHPGDNYTSYNQIFND
SEQ ID NO. 120: a protein sequence of CDR3 of the heavy chain of the 41C10E7 single-chain antibody
ARNYYGSLDY
SEQ ID NO. 121: a nucleic acid sequence of a hinge region of CD8α
ACCACGACGCCAGCGCCGCGACCACCAACACCGGCGCCCACCATCGCGTCGCAGCCCCTGTCCCTGC
GCCCAGAGGCGTGCCGGCCAGCGGCGGGGGGCGCAGTGCACACGAGGGGGCTGGACTTCGCCTGTGA
T
SEQ ID NO. 122: a protein sequence of the hinge region of CD8α
TTTPAPRPPTPAPTIASQPLSLRPEACRPAAGGAVHTRGLDFACD
SEQ ID NO. 123: a nucleic acid sequence of a transmembrane region of CD8α
ATCTACATCTGGGCGCCCTTGGCCGGGACTTGTGGGGTCCTTCTCCTGTCACTGGTTATCACCCTTTACT
GC
SEQ ID NO. 124: a protein sequence of the transmembrane region of CD8α
IYIWAPLAGTCGVLLLSLVITLYC
SEQ ID NO. 125: a nucleic acid sequence of a CD28 costimulatory factor
AGGAGTAAGAGGAGCAGGCTCCTGCACAGTGACTACATGAACATGACTCCCCGCCGCCCCGGGCCCA
CCCGCAAGCATTACCAGCCCTATGCCCCACCACGCGACTTCGCAGCCTATCGCTCC
SEQ ID NO. 126: a protein sequence of the CD28 costimulatory factor
RSKRSRLLHSDYMNMTPRRPGPTRKHYQPYAPPRDFAAYRS
SEQ ID NO. 127: a nucleic acid sequence of a 4-1BB costimulatory factor
AAACGGGGCAGAAAGAAACTCCTGTATATATTCAAACAACCATTTATGAGACCAGTACAAACTACTCAA
GAGGAAGATGGCTGTAGCTGCCGATTTCCAGAAGAAGAAGAAGGAGGATGTGAACTG
SEQ ID NO. 128: a protein sequence of the 4-1BB costimulatory factor
KRGRKKLLYIFKQPFMRPVQTTQEEDGCSCRFPEEEEGGCEL
SEQ ID NO. 129: a nucleic acid sequence of a CD3ζ signal transduction region
AGAGTGAAGTTCAGCAGGAGCGCAGACGCCCCCGCGTACCAGCAGGGCCAGAACCAGCTCTATAACG
AGCTCAATCTAGGACGAAGAGAGGAGTACGATGTTTTGGACAAGAGACGTGGCCGGGACCCTGAGAT
GGGGGGAAAGCCGAGAAGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAGGCCCTGCCCCCTCG
CTAA
SEQ ID NO. 130: a protein sequence of the CD3ζ signal transduction region
RVKFSRSADAPAYQQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR

### Example 4: Preparation of Retroviruses Containing the Chimeric Antigen Receptors

Moloney murine leukemia viruses (MMLV) were prepared by transiently transfecting 293T cells with a three-plasmid system (GFS-CAR, MMLV-gag-pol and RV-RD). CD19.28 CAR was used as a control, and B7-H3.BB CAR and B7-H3.28 CAR derived from a 15C11 humanized antibody were the CARs to be tested. After 293T cells were transfected for 48 and 72 h, supernatant of 293 T cells were collected and used to transduce CD3 and CD28 antibody-stimulated T cells from health individuals.

### Example 5: Preparation of T Cells Expressing the Chimeric Antigen Receptors Targeting B7-H3

1. T-Cell Transduction and Amplification

Peripheral blood mononuclear cells were isolated from peripheral blood donated by healthy individuals. The mononuclear cells were isolated by Lymphoprep density separation method. The isolated mononuclear cells were activated with CD3 and CD28 antibodies, and T cells were transduced with the retroviral supernatant (CD19.28 CAR, B7-H3.BB CAR, and B7-H3.28 CAR) collected in Example 4. The T cells transduced with the chimeric antigen receptors were further amplified for detection of the expression and tumor-killing effects of the B7-H3 chimeric antigen receptors.

2. Detection of the Expression of the B7-H3 Chimeric Antigen Receptor

3 days after transduction, B7-H3-targeted chimeric antigen receptor-T cells were collected, and the expression of the B7-H3-targeted chimeric antigen receptor was detected by using a fusion protein of B7-H3 and an antibody Fc fragment and a secondary goat-anti-human Fc antibody labelled with an APC fluorophore. The expression of the B7-H3 chimeric antigen receptors on the T cells was detected by flow cytometry.

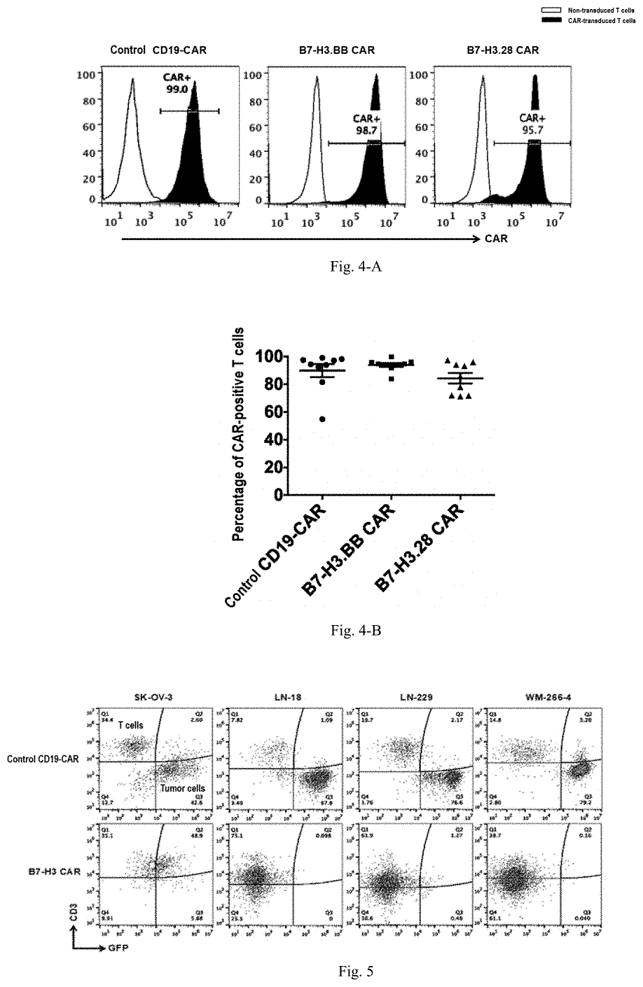

Results: similar to the control CD19.28 CAR, more than 90% of the T cells expressed B7-H3 chimeric antigen receptor (B7-H3.BB CAR or B7-H3.28 CAR) of the present disclosure (see FIG. 4A). The results were also confirmed in T cells from multiple different healthy donors (see FIG. 4B).

### Example 6: Killing Effects of B7-H3-Targeted Chimeric Antigen Receptor-T Cells on Tumor Cells in Co-Culture Systems

In Vitro

1. Killing Effect of the B7-H3-Targeted Chimeric Antigen Receptor-T Cells on Tumor Cells

To determine whether the chimeric antigen receptor of the present disclosure could mediate T cells to kill tumor cells highly expressing B7-H3, we designed a co-culture experiment of T cells and tumor cells to test the killing effects of the T cells on various tumor cells. After the tumor cell attachment for 24 h, T cells expressing the control (CD19.28 CAR-T) and the B7-H3-targeted chimeric antigen receptor (B7-H3.28 CAR-T) were added, and then killing effects of CAR-T cells on various tumor cells were detected by flow cytometry.

Method: tumor cells (SK-OV-3, LN-18, LN-229, and WM-266-4) labelled with GFP were inoculated into a 24-well plate with a density of 1-2×10⁵ cells per well, and 24 h later, a corresponding number of CAR-T cells transduced for 10 days were added according to a ratio of the T cells to the tumor cells of 1:1.7 d later, the cells in the wells were collected, and percentage of remaining tumor cells was detected by flow cytometry. The CAR-T cells were detected by using a mouse-anti-human CD3 fluorescent antibody, and the tumor cells were detected by using spontaneous GFP fluorescence.

Results: as shown in FIG. 5, the control CAR-T cells could not kill any kind of tumor cells, while the B7-H3 CAR-T cells of the present disclosure could completely kill the tumor cells when co-cultured with various tumor cell lines (SK-OV-3, LN-18, LN-229, and WM-266-4), and meanwhile, the CAR-T cells also exhibited cell proliferation.

2. Comparison of the B7-H3-Targeted CAR of the Present Disclosure and Other CARs Targeting the Same Antigen: The Affinity to the Antigen, and Cytokine Release During Tumor Cell Killing by Three Kinds of CAR-T Cells

In view of two existing other chimeric antigen receptors in the art, we compared the CAR of the present invention with the two CARs (CAR1 of WO2017/044699, and CAR2 of U.S. Pat. No. 10,233,226 B2) on the affinity to the B7-H3 antigen and cytokine release during tumor cell killing by the three kinds of CAR-T cells.

Method: Pull-Down Assay

293T cells were transfected with B7-H3-FLAG or FLAG empty vector control, respectively. Other 293T cells were transfected with the three kinds of B7-H3-targeted CARs, respectively, and the expression levels were adjusted to the same level. 48 h later, the 293T cells transfected with B7-H3-FLAG or FLAG empty vector control were lysed with RIPA lysis buffer and centrifuged, the supernatant was collected, an anti-FLAG M2 affinity gel was added, incubated at 4° C. for 2 h, and washed with RIPA lysis buffer for later use. The 293T cells transfected with different B7-H3-targeted CARs were lysed with RIPA lysis buffer and centrifuged, and supernatants were collected, and respectively incubated with the anti-FLAG M2 gel pull-down products of B7-H3-FLAG or FLAG empty vector control at 4° C. for 1 h. The products were then washed with RIPA lysis buffer for 4 times, eluted with elution buffer (0.1 M glycine-HCl, pH 3.5) by shaking at room temperature for 10 min, the supernatants were collected, neutralized with 1 M Tris-HCl (pH 7.5), added with an SDS loading buffer, and denatured at 95° C. for 5 min, and the affinity of the CARs to the antigen was analysed by western blot.

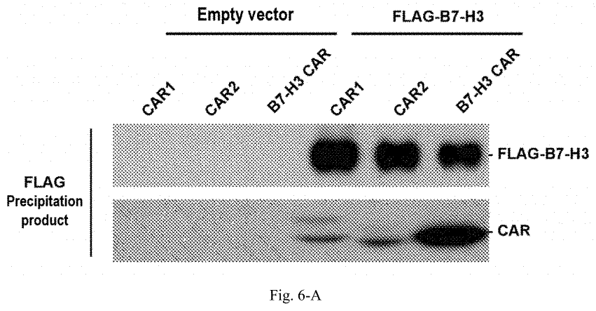

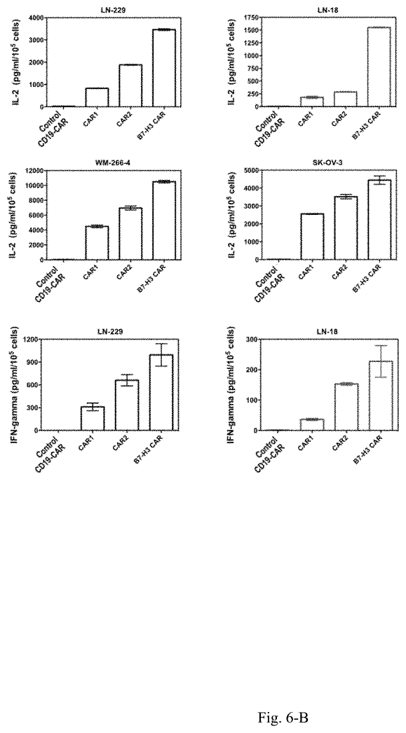

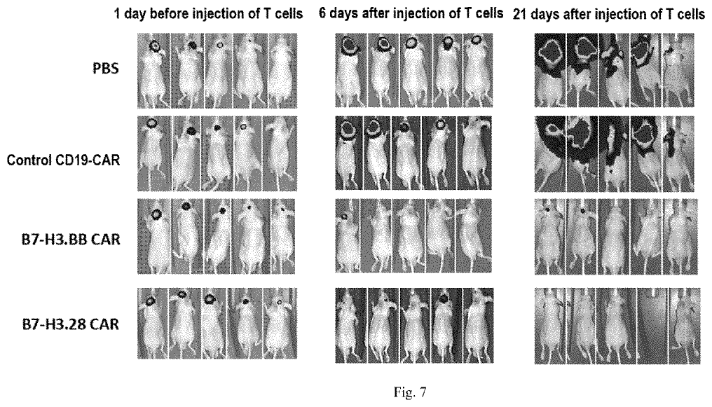

Results: as shown in FIG. 6A, the three kinds of CARs can bind to B7-H3, and compared with the other two kinds of CARs, the B7-H3-targeted CAR of the present disclosure can bind to more B7-H3 antigen proteins, which indicates that the CAR of the present disclosure has higher affinity to the antigen.

Method: enzyme-linked immunosorbent assay (ELISA) In the experiment of co-culture of four types of tumor cells (LN-229, LN-18, WM-266-4, and SK-OV-3) with the control CD19.28 CAR-T, CAR1, CAR2, and the B7-H3.28 CAR-T of the present disclosure, after adding the 10-day transduced T cells for 24 h, part of supernatant was collected. Cytokines in the supernatant were determined by using an ELISA kit.

Results: as shown in FIG. 6B that the levels of cytokines released by the control CD19.28 CAR-T cells during the co-culture with the tumor cells were below detection limit, while the three kinds of B7-H3-targeted CAR-T cells could release Th1 type cytokines (including IFNgamma, IL-2, etc.) during killing. Compared with CAR1 and CAR2, the B7-H3-targeted CAR-T of the present disclosure could obviously release higher levels of IL-2 and IFNgamma during killing the four types of tumor cells. Because the levels of these cytokines could greatly affect the final killing effect of CAR-T on tumors, the B7-H3-targeted CAR-T of the present disclosure showed significantly better killing effect on tumors compared with the two existing B7-H3-targeted CAR-T.

### Example 7: Experiment of the In Situ Tumor Xenograft Mouse Models

To determine the killing effect of the B7H3 CAR-T cells of the present invention on tumors in vivo, we chose nude mouse models of brain glioma. Human tumor cells (U-87MG or LN-229 cells) labelled with luciferase were injected into the brains of 6-8-week-old nude mice, and after tumors were grafted, the solvent PBS, the control CD19.28 CAR-T cells, the B7-H3.BB CAR-T cells, and the B7-H3.28 CAR-T cells were injected in situ, and the tumor-removal effects of the T cells and survival rates of the treated mice were detected by imaging.

Method: the in vivo experiment of CAR-T cells targeting glioma was completed in 6-8-week-old nude mice. Tumor cell lines (U-87MG cells) labelled with GFP-FFLuc were implanted into the brains of the nude mice through stereotaxis. 2 weeks later, the grafted tumors were determined in an in-vivo-imaging system, and the solvent PBS, and the CD19.28 CAR-T cells, the B7-H3.BB CAR-T cells and the B7-H3.28 CAR-T cells that were transduced for 10 days were injected in situ. Then, imaging was performed once a week to determine the killing effect of the T cells on the tumors.

In an in vivo survival experiment, LN-229 cells labelled with GFP-FFLuc were injected in situ into brains of mice through stereotaxis, and 2 weeks later, the solvent PBS, the control CD19.28 CAR-T cells, the B7-H3.BB CAR-T cells, and the B7-H3.28 CAR-T cells were injected in situ. Then, the body weight of the mice was measured weekly, and the time of death of the mice was recorded.

Results: as shown in FIG. 7, compared with the continuously growing tumors in the mice in the PBS group and the control CD19.28 CAR-T group, the B7-H3-targeted CAR-T cells (B7-H3.BB CAR-T and B7-H3.28 CAR-T) could remove the tumors formed by the U-87MG cells in most of the mice, and control tumor recurrence within a certain period. These results indicate that the T cells expressing the B7-H3-targeted chimeric antigen receptor can kill tumors in vivo.

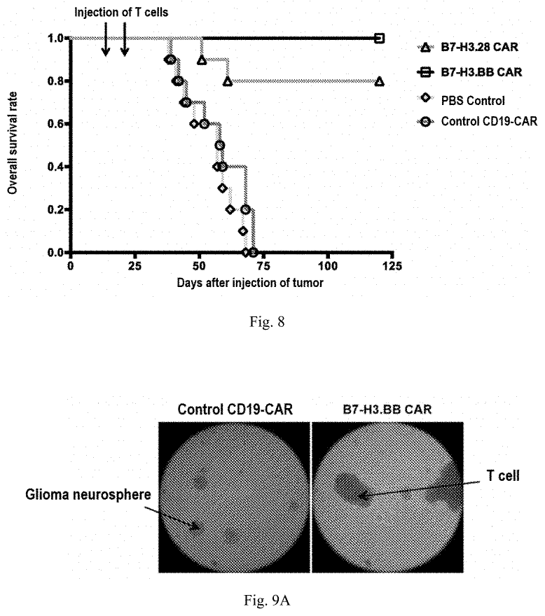

Meanwhile, we determined the survival rates of mice with glioma formed by LN-229 that were treated with the control and the T cells expressing the B7-H3-targeted chimeric antigen receptor. As shown in FIG. 8, compared with the control PBS and CD19.28 CAR-T, the T cells expressing the B7-H3-targeted chimeric antigen receptor (B7-H3.BB CAR-T and B7-H3.28 CAR-T) of the present disclosure significantly prolonged the survival time of mice, which indicate that the T cells expressing the B7-H3-targeted chimeric antigen receptor of the present disclosure can be used as a potential tumor therapy.

### Example 8: Killing Effects of the B7-H3-Targeted Chimeric Antigen Receptor-T Cells on Glioma from Patients

1. Killing Effect of the B7-H3-Targeted Chimeric Antigen Receptor-T Cells on Glioma Neurospheres from Patients in Vitro

In order to verify the killing effect of the T cells expressing the B7-H3-targeted chimeric antigen receptor of the present disclosure on tumors from patients, we isolated glioma stem cells from glioma patients and cultured them into glioma neurospheres. The glioma neurospheres were then co-cultured with the T cells expressing the control (CD19-CAR) and the T cells expressing the B7-H3-targeted chimeric antigen receptor (B7-H3.BB CAR or B7-H3.28 CAR), and then killing effects of the T cells on the glioma neurospheres of patients were observed by using a microscope and detected by flow cytometry.

Method: after glioma tissues of patients were obtained, glioma stem cells were isolated by using tissue dissociation kits, and cultured in glioma complete media to obtain glioma neurospheres. The glioma neurosphere cells of the patient were then inoculated into a 24-well plate according to a density of 1-2×10⁵ cells per well, and 24 h later, a corresponding number of CAR-T cells transduced for 10 days were added in a ratio of the T cells to the tumor cells of 1:1.5-7 days later, the cells in the wells were observed and collected, and percentage of the remaining tumor cells were determined by flow cytometry. The CAR-T cells were detected by using mouse-anti-human CD3 fluorescent antibodies, and the cells without labelling were glioma neurosphere cells.

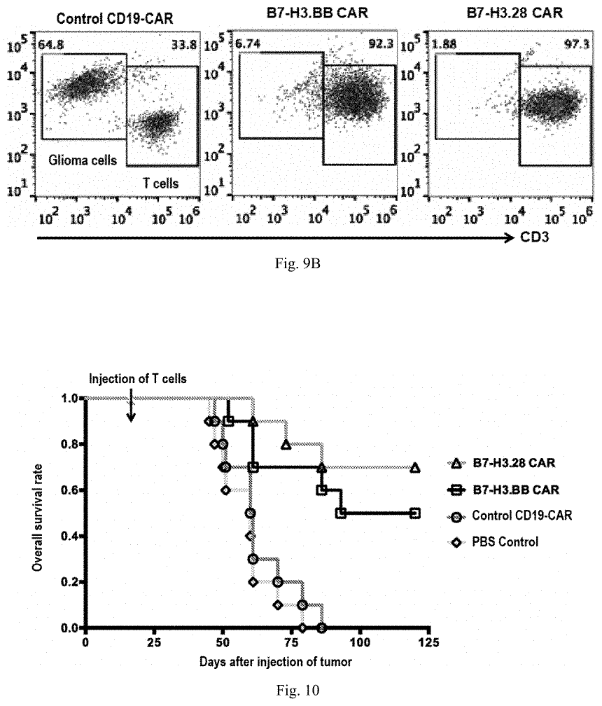

Results: as shown in FIG. 9, the control CD19.28 CAR-T cells could not kill the glioma neurospheres, while B7-H3.BB CAR-T and B7-H3.28 CAR-T of the present disclosure could effectively kill the glioma neurospheres, and the CAR-T cells exhibited obvious accumulation and proliferation.

2. Killing Effects of the B7-H3-Targeted Chimeric Antigen Receptor-T Cells on Glioma Neurospheres from Patients In Vivo

To further confirm in vivo killing effects of the T cells expressing the B7-H3-targeted chimeric antigen receptor on tumors from patients, we used the patient-derived glioma neurospheres to establish in situ tumor xenograft mouse model. The patient-derived glioma neurosphere cells were injected in situ into the brains of nude mice, and 2 weeks later, similarly, the solvent PBS, the control CD19.28 CAR-T cells, the B7-H3.BB CAR-T cell, and the B7-H3.28 CAR-T cells were injected into the brains, and then the survival rates of the treated mice in each group were recorded.

Method: an in vivo experiment of the CAR-T cells targeting patient-derived glioma neurospheres were completed in 6-8-week-old nude mice. Glioma neurosphere cells labelled with GFP-FFLuc were injected in situ into the brains of nude mice by using a stereotaxic instrument, and 2 weeks later, the solvent PBS, the control CD19.28 CAR-T cells, the B7-H3.BB CAR-T cells, and the B7-H3.28 CAR-T cells were injected in situ. Then, the body weight of the mice was measured weekly, and the time of death of the mice was recorded.

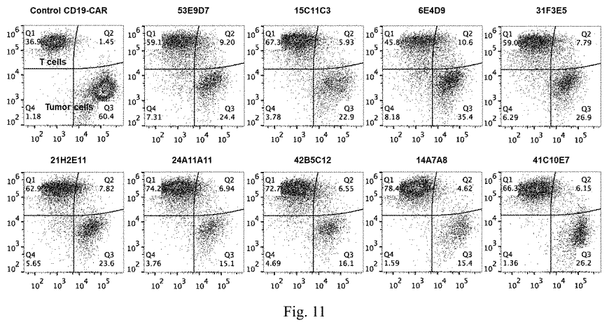

Results: as shown in FIG. 10, compared with the controls PBS and CD19.28 CAR-T, the T cells expressing the B7-H3-targeted chimeric antigen receptor (B7-H3.BB CAR-T and B7-H3. 28 CAR-T) of the present disclosure could significantly prolong the survival time of the tumor-bearing mice, which indicates that the T cells expressing the B7-H3-targeted chimeric antigen receptor of the present disclosure have the potential to effectively kill patient-derived glioma cells in vivo and prolong the survival time of patients.

### Example 9: Killing Effects of Other 9 Kinds of Murine-Derived B7-H3-Targeted Chimeric Antigen Receptor-T Cells on Tumor Cells in the In Vitro Co-Culture System

1. Killing Effects of 9 Kinds of Murine-Derived B7-H3-Targeted Chimeric Antigen Receptor-T Cells on Tumor Cells In addition to the humanized single-chain antibody cloned from 15C11, the present disclosure also includes other 9 murine-derived single-chain antibodies that recognize the B7-H3 antigen (named as: 53E9D7, 15C11C3, 6E4D9, 31F3E5, 21H2E11, 24A11A11, 42B5C12, 14A7A8, and 41C10E7, respectively). In order to determine that these murine-derived chimeric antigen receptors could also mediate T cells to kill tumor cells highly expressing B7-H3, we designed a co-culture experiment to test and compare killing effects of these murine-derived CAR-T cells on tumor cells (LN-229). After tumor cell attachment, we added the control CD19.28 CAR-T cells and the 9 kinds of murine-derived B7-H3.28 CAR-T cells, and then killing effects of the CAR-T cells on the tumor cells were determined by flow cytometry.

Method: LN-229 cells labelled with GFP were inoculated into a 24-well plate with a density of 2×10⁵ cells per well, and 24 h later, a corresponding number of the CD19.28 CAR-T cells and 9 kinds of different B7-H3.28 CAR-T cells transduced for 3 days were added according to a ratio of T cells to tumor cells of 1:1.5 days later, all cells in the wells were collected, and the percentage of the remaining LN-229 cells was determined by flow cytometry. T cells were detected by using mouse-anti-human CD3 fluorescent antibody to bind and stain, and the LN-229 cells were detected by spontaneous GFP fluorescence.

Results: as shown in FIG. 11, the control CD19.28 CAR-T cells did not show any obvious killing effect on the LN-229 cells, and the other 9 kinds of murine-derived B7-H3-targeted chimeric antigen receptor-T cells of the present disclosure showed killing effects on the tumor cells during the co-culture with the LN-229 cells, and meanwhile, the B7-H3-targeted CAR-T cells exhibited some proliferation.

2. Cytokine Release of the 9 Kinds of Murine-Derived B7-H3-Targeted Chimeric Antigen Receptor-T Cells During

Killing Tumor Cells In the co-culture experiment of the T cells and the tumor cells, we also detected the cytokine released by the 9 kinds of murine-derived B7-H3-targeted CAR-T cells during killing the tumor cells.

Method: Enzyme-Linked Immunosorbent Assay (ELISA)

The CAR-T cells (CD19.28 CAR-T and 9 kinds of murine-derived B7-H3.28 CAR-T) were added in the co-culture experiments with the tumor cells (LN-229 and WM-266-4), and 24 h later, a part of the culture supernatant was collected. Cytokines in the supernatant were detected by using an ELISA kit.

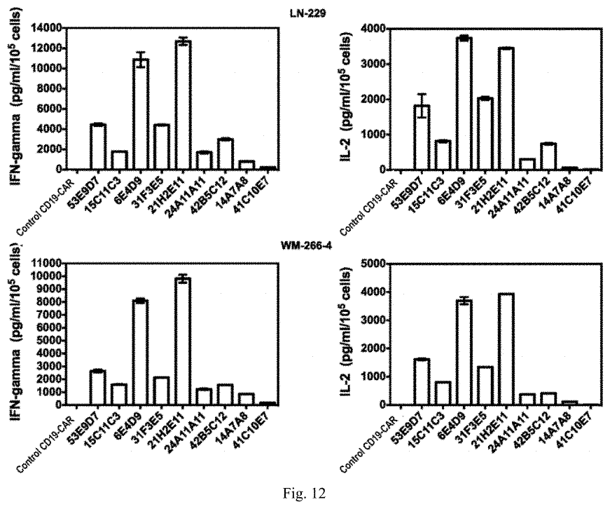

Results: As shown in FIG. 12 that the cytokines released by the control CD19.28 CAR-T cells were below the detection limit, and the 9 kinds of murine-derived B7-H3-targeted chimeric antigen receptor-T cells released different levels of Th1 type cytokines (including IFNgamma, IL-2, etc.) during killing the tumor cells, which indicates that the 9 kinds of CAR-T cells have the ability of killing tumor cells and activating other tumor killing mechanisms.

1. A B7-H3-targeting chimeric antigen receptor (CAR), characterized by comprising: an anti-B7-H3 binding domain, a hinge region, a transmembrane domain, and a signal transduction domain;
preferably, the anti-B7-H3 binding domain comprises an anti-B7-H3 single-chain antibody or antigen-binding portion, and the antibody or antigen-binding portion comprises a light-chain CDR selected from amino acid sequences of SEQ ID NO. 5-7, 17-19, 29-31, 41-43, 53-55, 65-67, 77-79, 89-91, 101-103, 113-115 or any variant thereof; and, comprises a heavy-chain CDR selected from amino acid sequences of SEQ ID NO. 10-12, 22-24, 34-36, 46-48, 58-60, 70-72, 82-84, 94-96, 106-108, 118-120 or any variant thereof.

2. (canceled)

3. The CAR according to claim 1, wherein the anti-B7-H3 binding domain comprises an anti-B7-H3 antibody or antigen-binding portion, and the antibody or antigen-binding portion comprises a combination of CDRs selected from the following light and heavy chains;
(1) light-chain CDR1, CDR2 and CDR3 sequences respectively comprising SEQ ID NO: 5-7, and heavy-chain CDR1, CDR2 and CDR3 sequences respectively comprising SEQ ID NO: 10-12;
(2) light-chain CDR1, CDR2 and CDR3 sequences respectively comprising SEQ ID NO: 17-19, and heavy-chain CDR1, CDR2 and CDR3 sequences respectively comprising SEQ ID NO: 22-24;
(3) light-chain CDR1, CDR2 and CDR3 sequences respectively comprising SEQ ID NO: 29-31, and heavy-chain CDR1, CDR2 and CDR3 sequences respectively comprising SEQ ID NO: 34-36;
(4) light-chain CDR1, CDR2 and CDR3 sequences respectively comprising SEQ ID NO: 41-43, and heavy-chain CDR1, CDR2 and CDR3 sequences respectively comprising SEQ ID NO: 46-48;
(5) light-chain CDR1, CDR2 and CDR3 sequences respectively comprising SEQ ID NO: 53-55, and heavy-chain CDR1, CDR2 and CDR3 sequences respectively comprising SEQ ID NO: 58-60;
(6) light-chain CDR1, CDR2 and CDR3 sequences respectively comprising SEQ ID NO: 65-67, and heavy-chain CDR1, CDR2 and CDR3 sequences respectively comprising SEQ ID NO: 70-72;
(7) light-chain CDR1, CDR2 and CDR3 sequences respectively comprising SEQ ID NO: 77-79, and heavy-chain CDR1, CDR2 and CDR3 sequences respectively comprising SEQ ID NO: 82-84;
(8) light-chain CDR1, CDR2 and CDR3 sequences respectively comprising SEQ ID NO: 89-91, and heavy-chain CDR1, CDR2 and CDR3 sequences respectively comprising SEQ ID NO: 94-96;
(9) light-chain CDR1, CDR2 and CDR3 sequences respectively comprising SEQ ID NO: 101-103, and heavy-chain CDR1, CDR2 and CDR3 sequences respectively comprising SEQ ID NO: 106-108; and
(10) light-chain CDR1, CDR2 and CDR3 sequences respectively comprising SEQ ID NO: 113-115, and heavy-chain CDR1, CDR2 and CDR3 sequences respectively comprising SEQ ID NO: 118-120.

4. The CAR according to claim 1, wherein the anti-B7-H3 binding domain comprises an anti-B7-H3 antibody or antigen-binding portion, and the antibody or antigen-binding portion comprises a light-chain sequence selected from amino acid sequences of SEQ ID NO.4, 16, 28, 40, 52, 64, 76, 88, 100, 112 or any variant thereof;
and, comprises a heavy-chain sequence selected from amino acid sequences of SEQ ID NO. 9, 21, 33, 45, 57, 69, 81, 93, 105, 117 or any variant thereof.

5. The CAR according to claim 1, wherein the anti-B7-H3 binding domain comprises an anti-B7-H3 antibody (scFv) domain; preferably, the anti-B7-H3 single-chain antibody has an amino acid sequence selected from amino acid sequences of SEQ ID NO. 2, 14, 26, 38, 50, 62, 74, 86, 98, 110 or any variant thereof.

6. The CAR according to claim 1, wherein the transmembrane domain is selected from one or more of transmembrane domains of α, β, ζ chains of TCR, CD3γ, CD3δ, CD3ε, CD3ζ, CD4, CD5, CD8α, CD9, CD16, CD22, CD27, CD28, CD33, CD3γ, CD45, CD64, CD80, CD86, CD134, 4-1BB, CD152, CD154, PD-1; preferably, the transmembrane domain is selected from one or more of transmembrane domains of CD8α, CD4, CD45, PD-1, CD154, CD28; preferably, the transmembrane domain is selected from one or more of transmembrane domains of CD8α or CD28; more preferably, the transmembrane domain has an amino acid sequence selected from amino acid sequences of SEQ ID NO. 124 or any variant thereof,
the hinge region is selected from one or more of an extracellular hinge region of CD8, a hinge region of IgG1 Fc CH2CH3, a hinge region of IgD, an extracellular hinge region of CD28, a hinge region of IgG4 Fc CH2CH3, and an extracellular hinge region of CD4; preferably, the hinge region is a hinge region of CD8α; more preferably, the hinge region has an amino acid sequence selected from amino acid sequences of SEQ ID NO. 122 or any variant thereof,
preferably, the signal transduction domain is selected from one or more of intracellular signaling regions of TCRζ, FcRγ, FcRβ, CD3γ, CD3δ, CD3ε, CD5, CD22, CD79a, CD79b, CD278 (ICOS), CD66d, CD3C preferably, the signal transduction domain is selected from an intracellular signaling region of CD33C more preferably, the signal transduction domain has an amino acid sequence selected from amino acid sequences of SEQ ID NO. 130 or any variant thereof;
preferably, the signal transduction domain further comprises one or more costimulatory domains; preferably, the costimulatory domain is selected from one or more of intracellular signaling regions of CARD11, CD2, CD7, CD27, CD28, CD30, CD40, CD54, CD83, OX40, 4-1BB, CD134, CD150, CD152, CD223, CD270, PD-L2, PD-L1, CD278, DAP10, LAT, NKD2C, SLP76, TRIM, FcεRIγ, MyD88, 4-1BB; preferably, the costimulatory domain is selected from intracellular signaling regions of 4-1BB, CD134, CD28, OX40; preferably, the costimulatory domain is selected from one or more of intracellular signaling regions of 4-1BB or CD28; more preferably, the costimulatory domain has an amino acid sequence selected from amino acid sequences of SEQ ID NO. 126, 128 or any variant thereof.

7. (canceled)

8. An isolated nucleic acid molecule, comprising a polynucleotide sequence encoding the CAR according to claim 1; preferably, the nucleic acid molecule has a nucleotide sequence selected from nucleotide sequences of SEQ ID NO. 1, 13, 25, 37, 49, 61, 73, 85, 97, 109 or any variant thereof.

9. A nucleic acid construct, comprising the nucleic acid molecule according to claim 8; preferably, the nucleic acid construct is a viral vector; more preferably, the viral vector is one or more of a retroviral vector, a lentiviral vector, an adenoviral vector, an adeno-associated viral vector.

10. A virus, comprising the nucleic acid molecule according to claim 8; preferably, the virus is one or more of a retrovirus, a lentivirus, an adenovirus, an adeno-associated virus.

11. A method for preparation of a genetically modified immune cell targeting tumor cells expressing B7-H3, comprising: a step of obtaining the CAR according to claim 1.

12. An isolated host cell, expressing the CAR according to claim 1; preferably, the host cell is a mammalian cell; more preferably, the host cell is one or more of a T cell, an NK cell or cell line, a 76T cell, an NKT cell, a macrophage or cell line, a PG13 cell line; more preferably, the host cell is a T cell; the most preferably, the host cell is a primarily cultured T cell.

13. The host cell according to claim 12, wherein the host cell further expresses other effector molecules, the other effector molecules comprise but are not limited to a cytokine, another CAR, a chemokine receptor, an siRNA reducing the expression of PD-1 or a protein blocking PD-L1, a TCR or a safety switch;
preferably, the cytokine is selected from one or more of TNF-α, TNF-β, VEGF, TPO, NGF-β, PDGF, TGF-α, TGF-β, IGF-I, IGF-II, EPO, M-CSF, IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, an interferon, an angiostatin, a thrombospondin, an endostatin;
preferably, the chemokine receptor is selected from one or more of CCR2, CCR5, CXCR2, CXCR4;
preferably, the safety switch is selected from one or more of HSVTK, VZVTK, iCaspase-9, iCaspase-1, iCaspase-8, truncated EGFR, RQR8.

14. A pharmaceutical composition, comprising the CAR according to claim 1.

15. A method for disease diagnosis, treatment or prevention in a subject, comprising: a step of obtaining the CAR according to claim 1, wherein the disease is selected from one or more of cancer, an inflammatory disease, an autoimmune disease;
preferably, the disease is a tumor expressing B7-H3; more preferably, the disease is selected from one or more of brain cancer, pancreatic cancer, ovarian cancer, kidney cancer, bladder cancer, skin cancer, stomach cancer, intestinal cancer, head and neck cancer, thyroid cancer, prostate cancer, lung cancer, and Kaposi's sarcoma; more preferably, the brain cancer is selected from one or more of glioblastoma, astrocytoma, meningioma, oligodendroglioma, neuroglioma.

16. A method for preparation of a drug, comprising: a step of obtaining the CAR according to claim 1, preferably, the drug is used to diagnose, treat or prevent one or more of cancer, an inflammatory disease, an autoimmune disease;
preferably, the drug is used to diagnose, treat or prevent a tumor expressing B7-H3; more preferably, the drug is used to diagnose, treat or prevent one or more selected from brain cancer, pancreatic cancer, ovarian cancer, kidney cancer, bladder cancer, skin cancer, stomach cancer, intestinal cancer, head and neck cancer, thyroid cancer, prostate cancer, lung cancer, and Kaposi's sarcoma; more preferably, the brain cancer is selected from one or more of glioblastoma, astrocytoma, meningioma, oligodendroglioma, neuroglioma.

17. A method for preparation of a genetically modified immune cell targeting tumor cells expressing B7-H3, comprising: a step of obtaining of the nucleic acid molecule according to claim 8.

18. An isolated host cell, comprising the isolated nucleic acid molecule according to claim 8; preferably, the host cell is a mammalian cell; more preferably, the host cell is one or more of a T cell, an NK cell or cell line, a 76T cell, an NKT cell, a macrophage or cell line, a PG13 cell line; more preferably, the host cell is a T cell; the most preferably, the host cell is a primarily cultured T cell.

19. The host cell according to claim 18, wherein the host cell further expresses other effector molecules, the other effector molecules comprise but are not limited to a cytokine, another CAR, a chemokine receptor, an siRNA reducing the expression of PD-1 or a protein blocking PD-L1, a TCR or a safety switch;
preferably, the cytokine is selected from one or more of TNF-α, TNF-β, VEGF, TPO, NGF-β, PDGF, TGF-α, TGF-β, IGF-I, IGF-II, EPO, M-CSF, IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, an interferon, an angiostatin, a thrombospondin, an endostatin;
preferably, the chemokine receptor is selected from one or more of CCR2, CCR5, CXCR2, CXCR4;
preferably, the safety switch is selected from one or more of HSVTK, VZVTK, iCaspase-9, iCaspase-1, iCaspase-8, truncated EGFR, RQR8.

20. A pharmaceutical composition, comprising the nucleic acid molecule according to claim 8.

21. A method for disease diagnosis, treatment or prevention in a subject, comprising: a step of obtaining a nucleic acid molecule encoding the CAR according to claim 1, wherein the disease is selected from one or more of cancer, an inflammatory disease, an autoimmune disease;
preferably, the disease is a tumor expressing B7-H3; more preferably, the disease is selected from one or more of brain cancer, pancreatic cancer, ovarian cancer, kidney cancer, bladder cancer, skin cancer, stomach cancer, intestinal cancer, head and neck cancer, thyroid cancer, prostate cancer, lung cancer, and Kaposi's sarcoma; more preferably, the brain cancer is selected from one or more of glioblastoma, astrocytoma, meningioma, oligodendroglioma, neuroglioma.

22. A method for preparation of a drug, comprising: a step of obtaining the nucleic acid molecule according to claim 8; preferably, the drug is used to diagnose, treat or prevent one or more of cancer, an inflammatory disease, an autoimmune disease;
preferably, the drug is used to diagnose, treat or prevent a tumor expressing B7-H3; more preferably, the drug is used to diagnose, treat or prevent one or more selected from brain cancer, pancreatic cancer, ovarian cancer, kidney cancer, bladder cancer, skin cancer, stomach cancer, intestinal cancer, head and neck cancer, thyroid cancer, prostate cancer, lung cancer, and Kaposi's sarcoma; more preferably, the brain cancer is selected from one or more of glioblastoma, astrocytoma, meningioma, oligodendroglioma, neuroglioma.
